(19) United States
(12) Patent Application Publication
(10) Pub. No.: US 2025/0185454 A1
(43) Pub. Date: Jun. 5, 2025

(54) COMPOUND, LIGHT-EMITTING MATERIAL, AND LIGHT-EMITTING ELEMENT

(71) Applicant: KYULUX, INC., Fukuoka-shi, Fukuoka (JP)

(72) Inventors: Masataka YAMASHITA, Fukuoka-shi, Fukuoka (JP); Yoshitake SUZUKI, Fukuoka-shi, Fukuoka (JP); Takuya HIGA, Fukuoka-shi, Fukuoka (JP); Yuta WATABIKI, Fukuoka-shi, Fukuoka (JP); Momoko MORIO, Fukuoka-shi, Fukuoka (JP); Umamahesh BALIJAPALLI, Fukuoka-shi, Fukuoka (JP); Kousei KANAHARA, Fukuoka-shi, Fukuoka (JP); Yuba Raj GAIHRE, Fukuoka-shi, Fukuoka (JP); Hiroaki OZAWA, Fukuoka-shi, Fukuoka (JP)

(21) Appl. No.: 18/843,023

(22) PCT Filed: Jan. 24, 2023
(86) PCT No.: PCT/JP2023/002022

§ 371 Date: Aug. 30, 2024

(30) Foreign Application Priority Data
Mar. 2, 2022 (JP) 2022-032152

(51) Int. Cl. H10K 50/12; C07D 491/052; C07D 495/04; H10K 85/30; H10K 85/40; H10K 85/60; H10K 101/20

(52) U.S. Cl. CPC H10K 50/12; C07D 491/052; C07D 495/04; H10K 85/324; H10K 85/371; H10K 85/40; H10K 85/622; H10K 85/624; H10K 85/633; H10K 85/636; H10K 85/654; H10K 85/656; H10K 85/6572; H10K 85/6574; H10K 85/6576; H10K 85/658; H10K 2101/20

(57) ABSTRACT

An organic light emitting device using a compound of the following general formula has excellent light emission characteristics. In the following general formula, 1 to 2 of R¹ to R⁴ each are a ring-fused carbazol-9-yl group, and at least one of them is a ring-fused carbazol-9-yl group substituted with at least two substituted or unsubstituted aryl groups; 2 to 3 of R¹ to R⁴ each are a non-ring-fused carbazol-9-yl group.

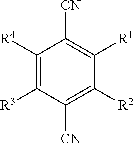

### TECHNICAL FIELD

The present invention relates to a compound useful as a light emitting material, and a light emitting device using the compound.

### BACKGROUND ART

Studies for enhancing the light emission efficiency of light emitting devices such as organic electroluminescent devices (organic EL devices) are being made actively. In particular, various kinds of efforts have been made for increasing light emission efficiency by newly developing and combining an electron transporting material, a hole transporting material, and a light-emitting material to constitute an organic electroluminescent device. Among them, there are seen some reports relating to an organic electroluminescent device that utilizes a delayed fluorescent material.

A delayed fluorescent material is a material which, in an excited state, after having undergone reverse intersystem crossing from an excited triplet state to an excited singlet state, emits fluorescence when returning back from the excited singlet state to a ground state thereof. Fluorescence through the route is observed later than fluorescence from the excited singlet state directly occurring from the ground state (ordinary fluorescence), and is therefore referred to as delayed fluorescence. Here, for example, in the case where a light emitting compound is excited through carrier injection thereinto, the occurring probability of the excited singlet state to the excited triplet state is statistically 25%/75%, and therefore improvement of light emission efficiency by the fluorescence alone from the directly occurring excited singlet state is limited. On the other hand, in a delayed fluorescent material, not only the excited singlet state thereof but also the excited triplet state can be utilized for fluorescent emission through the route via the above-mentioned reverse intersystem crossing, and therefore as compared with an ordinary fluorescent material, a delayed fluorescent material can realize a higher emission efficiency.

Since such a principle has been clarified, various studies have led to the discovery of various delayed fluorescent materials. They include, for example, the following compound in which benzene is substituted with two cyano groups and four substituted or unsubstituted carbazol-9-yl groups (PTL 1).

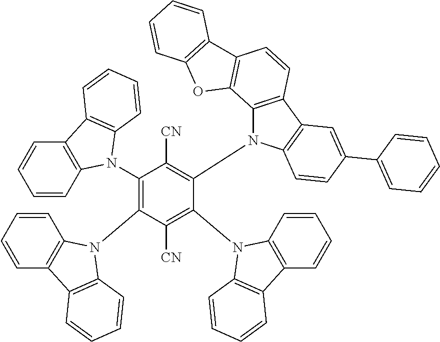 [SMILES: N#Cc1c(-n2c3ccccc3c3ccccc32)c(-n2c3ccccc3c3ccccc32)c(C#N)c(-n2c3ccc(-c4ccccc4)cc3c3ccc4c5ccccc5oc4c32)c1-n1c2ccccc2c2ccccc21]

### CITATION LIST

### Patent Literature

PTL 1: WO2020/022378

### SUMMARY OF INVENTION

### Technical Problem

Even if a material emits delayed fluorescence, one having extremely good characteristics and having no problem in practical use has not been provided. Therefore, for example, it is more useful if it is possible to provide a delayed fluorescent material having much better light emission characteristics than the delayed fluorescent material proposed in PTL 1. However, the improvement of delayed fluorescent materials is in the stage of trial and error, and it is not easy to generalize the chemical structure of useful light emitting materials.

Under such circumstances, the present inventors have conducted research for the purpose of providing a compound more useful as a light emitting material for a light emitting device. Then, the present inventors have conducted intensive studies for the purpose of deriving and generalizing a general formula of a compound more useful as a light emitting material.

### Solution to Problem

As a result of intensive studies for achieving the above object, the present inventors have found that a dicyanobenzene compound having a structure satisfying a specific condition is useful as a light emitting material. The present invention has been proposed based on these findings, and specifically has the following configuration.

[1] A compound represented by the following general formula (1).

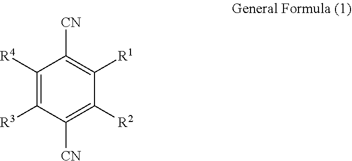

In the general formula (1), R¹ and 0 or 1 of R² to R⁴ each independently represent a substituted or unsubstituted ring-fused carbazol-9-yl group,
at least R¹ among them is a ring-fused carbazol-9-yl group in which the ring skeleton-constituting carbon atoms are substituted with at least two substituted or unsubstituted aryl groups, and
the remaining R² to R⁴ each independently represent a substituted or unsubstituted non-ring-fused carbazol-9-yl group.

[2] The compound according to [1], wherein, when one of R² to R⁴ is a ring-fused carbazol-9-yl group, the ring skeleton-constituting carbon atoms of the ring-fused carbazol-9-yl group are substituted with at least two substituted or unsubstituted aryl groups.

[3] The compound according to [1], wherein R¹ represents a ring-fused carbazol-9-yl group in which the ring skeleton-constituting carbon atoms are substituted with at least two substituted or unsubstituted aryl groups, and R² to R⁴ each independently represent a substituted or unsubstituted non-ring-fused carbazol-9-yl group.

[4] The compound according to any one of [1] to [3], wherein the ring-fused carbazol-9-yl group in which the ring skeleton-constituting carbon atoms are substituted with at least two substituted or unsubstituted aryl groups has a 5- to 7-membered ring-fused structure including carbazole.

[5] The compound according to any one of [1] to [4], wherein the ring-fused carbazol-9-yl group in which the ring skeleton-constituting carbon atoms are substituted with at least two substituted or unsubstituted aryl groups has a structure represented by the following general formula (2).

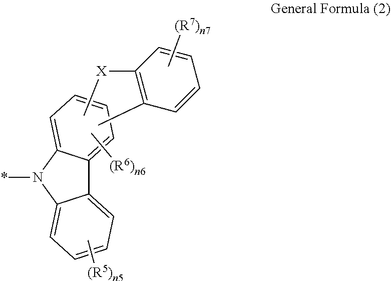

In the general formula (2), X represents O, S, N(R⁸) or C(R⁹)(R¹⁰). R⁵ to R⁷ each independently represent a substituent, in which the substituent includes a deuterium atom,
n5 and n7 each independently represent an integer of 0 to 4, and n6 represents an integer of 0 to 2,
n5+n6+n7 is an integer of 2 to 10, and at least two of R⁵ to R⁷ existing in the molecule are substituted or unsubstituted aryl groups,
R⁵'s bonding to the neighboring ring skeleton-constituting carbon atoms, R⁶'s bonding to the neighboring ring skeleton-constituting carbon atoms, and R⁷'s bonding to the neighboring ring skeleton-constituting carbon atoms each can bond to each other to form a cyclic structure, and
* indicates the bonding site to the benzene ring in the general formula (1).

[6] The compound according to [5], wherein n5+n6+n7 is 2.

[7] The compound according to [5] or [6], wherein:
only one of R⁵ and one of R⁷ each are independently a substituted or unsubstituted aryl group, or
only one of R⁵ and one of R⁶ each are independently a substituted or unsubstituted aryl group, or
only one of R⁶ and one of R⁷ each are independently a substituted or unsubstituted aryl group.

[8] The compound according to any one of [1] to [7], wherein the substituted or unsubstituted non-ring-fused carbazol-9-yl group has a structure represented by the following general formula (3).

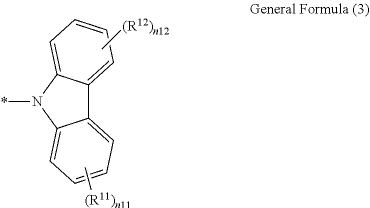

In the general formula (3), R¹¹ and R¹² each independently represent a substituent, in which the substituent includes a deuterium atom,
n11 and n12 each independently represent an integer of 0 to 4
one of the 1- to 4-positioned ring skeleton-constituting carbon atoms of the carbazole can be substituted with a nitrogen atom, and one of the 5- to 8-positioned ring skeleton-constituting carbon atoms of the carbazole can be substituted with a nitrogen atom, and
* indicates the bonding site to the benzene ring in the general formula (1).

[9] A composition containing the compound of any one of [1] to [8] and a pyrromethene-boron complex compound.

[10] A light emitting material including the compound of any one of [1] to [8].

[11] A delayed fluorescent material including the compound of any one of [1] to [8].

[12] A film including the compound according to any one of [1] to [8], or the composition according to [9].

[13] An organic semiconductor device including the compound of any one of [1] to [8] or the composition of [9].

[14] An organic light emitting device including the compound of any one of [1] to [8] or the composition of [9].

[15] The organic light emitting device according to [14], in which the device has a layer containing the compound, and the layer also contains a host material.

[16] The organic light emitting device according to [15], in which the layer containing the compound further contains a delayed fluorescent material in addition to the compound and the host material, and the delayed fluorescent material has a lowest excited singlet energy lower than that of the host material and higher than that of the compound.

[17] The organic light emitting device according to [15], in which the device has a layer containing the compound, and the layer also contains a light emitting material having a structure different from that of the compound.

[18] The organic light emitting device according to any one of [15] to [17], in which the amount of light emitted from the compound is the largest among the materials contained in the device.

[19] The organic light emitting device according to [17], in which the amount of light emitted from the light emitting material is larger than the amount of light emitted from the compound.

[20] The organic light emitting device according to any one of [14] to [19], which is an organic electroluminescent device.

[21] The organic light emitting device according to any one of [14] to [20], which emits delayed fluorescence.

### Advantageous Effects of Invention

The compound of the present invention is useful as a light emitting material. The compound of the present invention includes compounds having good light emission characteristics. Further, the organic light emitting device using the compound of the present invention also includes excellent devices having a high light emission efficiency, and useful devices having a long device lifetime.

### DESCRIPTION OF EMBODIMENTS

The contents of the invention will be described in detail below. The constitutional elements may be described below with reference to representative embodiments and specific examples of the invention, but the invention is not limited to the embodiments and the specific examples. In the description herein, a numerical range expressed as “to” means a range that includes the numerical values described before and after “to” as the lower limit and the upper limit. A part or all of hydrogen atoms existing in the molecule of the compound for use in the present invention can be substituted with deuterium atoms (²H, deuterium D). In the chemical structural formulae in the description herein, the hydrogen atom is expressed as H, or the expression thereof is omitted. For example, when expression of the atoms bonding to the ring skeleton-constituting carbon atoms of a benzene ring is omitted, H is considered to bond to the ring skeleton-constituting carbon atom at the site having the omitted expression. In the chemical structural formulae in the present description, a deuterium atom is expressed as D. The term “light emission characteristics” used in the present application refers to properties relating to light emission, such as light emission efficiency, drive voltage, and light emission lifetime. The compound represented by the general formula (1) is excellent in at least one light emission characteristic.

### [Compound Represented by General Formula (1)]

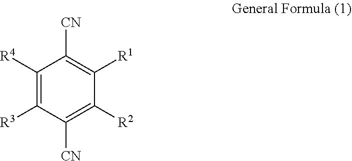

In the general formula (1), R¹ and 0 or 1 of R² to R⁴ each independently represent a ring-fused carbazol-9-yl group.

The ring-fused carbazol-9-yl group as referred to herein is a group that bonds via the nitrogen atom constituting the carbazole ring, and has a structure in which a ring is fused to at least one of the two benzene rings constituting the carbazol-9-yl group. In the ring-fused carbazol-9-yl group, the number of the rings that constitute the carbazole-containing fused ring is 4 or more, preferably 5 to 9, and more preferably 5 to 7. In one preferred aspect of the present invention, the number of the rings that constitute the carbazole-containing fused ring is 5.

The ring fused to the carbazol-9-yl group is one ring selected from an aromatic hydrocarbon ring, an aromatic heterocyclic ring, an aliphatic hydrocarbon ring and an aliphatic heterocyclic ring, or a ring formed by fusing at least two of the rings. In the case of fusing at least two, the ring can be one formed by fusing at least two rings of the same type, or can be one formed by fusing at least two rings of different types. An example of the former is a naphthalene ring formed by fusing two benzene rings, and an example of the latter is a benzofuran ring formed by fusing a benzene ring and a furan ring. Preferred examples of the fused ring include one ring selected from the group consisting of an aromatic hydrocarbon ring and an aromatic heterocyclic ring, and a ring formed by fusing at least two of such rings.

The aromatic hydrocarbon ring includes a benzene ring. The aromatic heterocyclic ring means a ring exhibiting aromaticity including a heteroatom as a ring skeleton-constituting atom, and is preferably a 5- to 7-membered ring, and for example, a 5-membered ring or a 6-membered ring can be employed. In one aspect of the present invention, a furan ring, a thiophene ring, or a pyrrole ring can be employed as the aromatic heterocyclic ring. It is preferable that a substituent selected from Substituent Group E bonds to the nitrogen atom of the pyrrole ring, and it is more preferable that an aryl group which can be substituted with an alkyl group or an aryl group bonds thereto. Preferably, the aromatic heterocyclic ring includes a furan ring and a thiophene ring. The aliphatic hydrocarbon ring includes a cyclopentadiene ring.

In one aspect of the present invention, the ring fusing to the carbazol-9-yl group is selected from a ring selected from the group consisting of a benzene ring, a furan ring, a thiophene ring, a pyrrole ring and a cyclopentadiene ring, or a ring formed by fusing at least two such rings. In one preferred aspect of the present invention, the ring is selected from a ring selected from the group consisting of a benzene ring, a furan ring, a thiophene ring and a pyrrole ring, or a ring formed by fusing at least two such rings. In one more preferred aspect of the present invention, the ring is selected from a ring selected from the group consisting of a benzene ring, a furan ring and a thiophene ring, or a ring formed by fusing at least two such rings.

In one aspect of the present invention, the ring fusing to the carbazol-9-yl group is a benzene ring, a naphthalene ring, a benzofuran ring, a benzothiophene ring, an indole ring or an indene ring. In one preferred aspect of the present invention, the ring fusing to the carbazol-9-yl group is a benzene ring, a naphthalene ring, a benzofuran ring or a benzothiophene ring. In one more preferred aspect of the present invention, the ring fusing to the carbazol-9-yl group is a benzene ring, a benzofuran ring or a benzothiophene ring. In one especially preferred aspect of the present invention, the ring fusing to the carbazol-9-yl group is a benzofuran ring or a benzothiophene ring. The benzofuran ring, the benzothiophene ring, the indole ring and the indene ring as referred to herein are 5-membered rings to fuse with the benzene ring that constitutes the carbazol-9-yl group.

A ring may fuse at one to four sites in the carbazol-9-yl group, but preferably fuses at one or two sites, and more preferably at one site. In the case where rings fuse at two or more sites, the fusing rings can be the same or different, but are preferably the same.

In the present invention, as the ring-fused carbazol-9-yl group, especially preferably employed is a benzofuran ring-fused carbazol-9-yl group, or a benzothiophene ring-fused carbazol-9-yl group.

In the present invention, a benzofuro[2,3-a]carbazol-9-yl group can be employed as the benzofuran ring-fused carbazol-9-yl group. Also, a benzofuro[3,2-a]carbazol-9-yl group can be employed. Also, a benzofuro[2,3-b]carbazol-9-yl group can be employed. Also, a benzofuro[3,2-b]carbazol-9-yl group can be employed. Also, a benzofuro[2,3-c]carbazol-9-yl group can be employed. Also, a benzofuro[3,2-c]carbazol-9-yl group can be employed. These structures are as described below, and at least one hydrogen atom in the following structures can be substituted. The benzene ring in the following structures can be further fused with a ring, but preferably the benzene ring in the following structures is not fused with a ring.

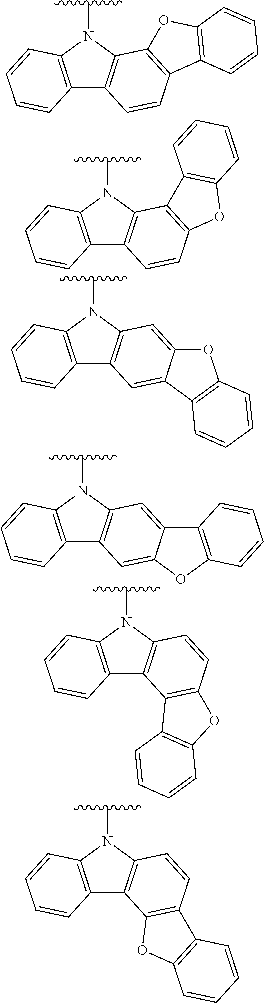

In the present invention, as the benzothiophene ring-fused carbazol-9-yl group, a benzothieno[2,3-a]carbazol-9-yl group can be employed. Also, a benzothieno[3,2-a]carbazol-9-yl group can be employed. Also, a benzothieno[2,3-b]carbazol-9-yl group can be employed. Also, a benzothieno[3,2-b]carbazol-9-yl group can be employed. Also, a benzothieno[2,3-c]carbazol-9-yl group can be employed. Also, a benzothieno[3,2-c]carbazol-9-yl group can be employed. These structures are as described below, and at least one hydrogen atom in the following structures can be substituted. The benzene ring in the following structures can be further fused with a ring, but preferably the benzene ring in the following structures is not fused with a ring.

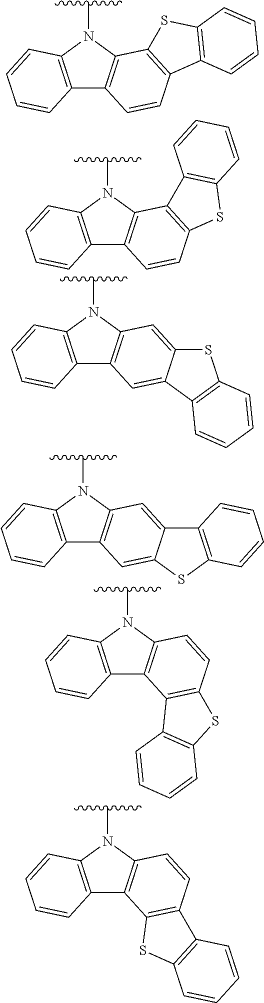

R¹ in the general formula (1) is a ring-fused carbazol-9-yl group in which the ring skeleton-constituting carbon atoms are substituted with at least two substituted or unsubstituted aryl groups. When one of R² to R⁴ is a ring-fused carbazol-9-yl group, the ring-fused carbazol-9-yl group can be such that the ring skeleton-constituting carbon atoms of the ring-fused carbazol-9-yl group are substituted with at least two substituted or unsubstituted aryl groups. In that case, the ring-fused carbazol-9-yl group, which R¹ and one of R² to R⁴ represent and in which the ring skeleton-constituting carbon atoms are substituted with at least two substituted or unsubstituted aryl groups, can be the same as or different from each other. Preferably, the groups are the same.

The number of the substituted or unsubstituted aryl groups with which the ring-fused carbazol-9-yl group is substituted is preferably 2 to 10, more preferably 2 to 6, even more preferably 2 to 4, and further more preferably 2 or 3. In one preferred aspect of the present invention, the number of the substituted or unsubstituted aryl groups is 2. The substituted or unsubstituted aryl groups with which the ring-fused carbazol-9-yl group is substituted can be the same as or different from each other. In one aspect of the present invention, all the substituted or unsubstituted aryl groups with which the ring-fused carbazol-9-yl group is substituted are the same.

The “aryl group” can be a monocyclic ring or a fused ring in which at least two rings are fused. In the case of a fused ring, the number of fused rings is preferably 2 to 6, and can be selected from, for example, 2 to 4. Specific examples of the ring include a benzene ring, a naphthalene ring, an anthracene ring, a phenanthrene ring, and a triphenylene ring. In one aspect of the present invention, the aryl group is a substituted or unsubstituted phenyl group, a substituted or unsubstituted naphthalen-1-yl group, or a substituted or unsubstituted naphthalen-2-yl group, and is preferably a substituted or unsubstituted phenyl group. For example, the substituent for the aryl group can be selected from Substituent Group A, can be selected from Substituent Group B, can be selected from Substituent Group C, can be selected from Substituent Group D, or can be selected from Substituent Group E. In one aspect of the present invention, the substituent for the aryl group is one group selected from the group consisting of an alkyl group, an aryl group and a deuterium atom, or a group formed by combining at least two such groups. In one preferred aspect of the present invention, the aryl groups with which the ring-fused carbazol-9-yl group is substituted are unsubstituted.

Specific examples of the substituted or unsubstituted aryl groups with which the ring-fused carbazol-9-yl group can be substituted are shown below. However, the substituted or unsubstituted aryl groups which can be employed in the present invention shall not be construed as being limited by the following specific examples. In the following specific examples, * indicates a bonding site to the ring-fused carbazole skeleton. In the following specific examples, expression of a methyl group is omitted. Accordingly, Ar4 is substituted by a methyl group, and Ar5 is substituted by an isopropyl group. * indicates a bonding site.

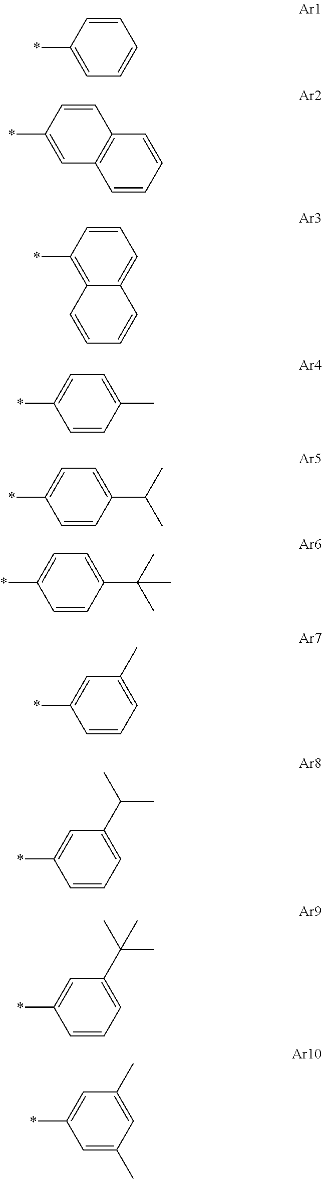

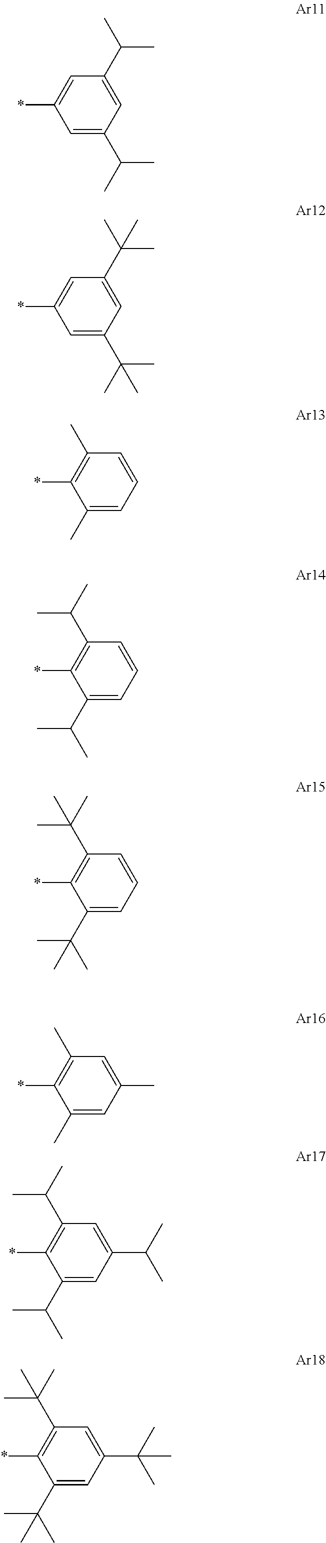

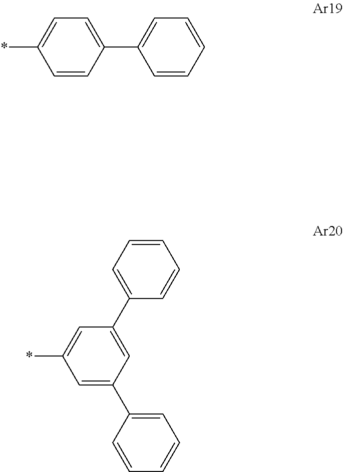 [SMILES: *c1ccc(-c2ccccc2)cc1 ; *c1cc(-c2ccccc2)cc(-c2ccccc2)c1]

Groups obtained by substituting all hydrogen atoms present in the above Ar1 to Ar20 with deuterium atoms are disclosed as Ar21 to Ar40, respectively. Groups obtained by substituting all hydrogen atoms present in the phenyl group or the alkyl group which is the substituent in the above Ar4 to Ar20 with deuterium atoms are disclosed as Ar41 to Ar57, respectively.

The substitution position of the substituted or unsubstituted aryl group bonding to the ring-fused carbazol-9-yl group is not specifically limited. In one aspect of the present invention, the ring skeleton-constituting carbon atom of each ring to constitute the ring-fused carbazol-9-yl group is substituted with 0 to 2 substituted or unsubstituted aryl groups, and preferably, the ring skeleton-constituting carbon atom of each ring to constitute the ring-fused carbazol-9-yl group is substituted with 0 to 1 substituted or unsubstituted aryl group. In one preferred aspect of the present invention, at least one substituted or unsubstituted aryl group is substituted at the para-position of the benzene ring from the heteroatom constituting the ring skeleton of the ring-fused carbazol-9-yl group. In one preferred aspect of the present invention, a substituted or unsubstituted aryl group is substituted only at the para-position of the benzene ring from the heteroatom constituting the ring skeleton of the ring-fused carbazol-9-yl group, and the other ring skeleton-constituting carbon atom is not substituted with a substituted or unsubstituted aryl group. The substituted or unsubstituted aryl group bonding to the ring skeleton-constituting hetero atom (for example, nitrogen atom) of the ring-fused carbazol-9-yl group is not counted in the number of the substituted or unsubstituted aryl groups bonding to the ring skeleton-constituting carbon atoms as referred to herein.

The ring skeleton-constituting carbon atoms of the ring-fused carbazol-9-yl group can be substituted with any substituents other than at least two substituted or unsubstituted aryl groups. For example, they can be further substituted with a substituent selected from Substituent Group A (excluding substituted or unsubstituted aryl group), or can be further substituted with a substituent selected from Substituent Group B (excluding substituted or unsubstituted aryl group), or can be further substituted with a substituent selected from Substituent Group C (excluding substituted or unsubstituted aryl group), or can be further substituted with a substituent selected from Substituent Group D (excluding substituted or unsubstituted aryl group), or can be further substituted with a substituent selected from Substituent Group E (excluding substituted or unsubstituted aryl group). In one aspect of the present invention, they can be further substituted with an alkyl group optionally substituted with a deuterium atom or an aryl group, or a deuterium atom, or with both the two. In one aspect of the present invention, they can be further substituted with a deuterium atom. In one aspect of the present invention, the ring skeleton-constituting carbon atom of the ring-fused carbazol-9-yl group is not substituted with any other substituent than at least two substituted or unsubstituted aryl groups.

The “alkyl group” as referred to herein can be linear, branched or cyclic, but is preferably linear or branched. At least two of a linear moiety, a cyclic moiety and a branched moiety can be in the group as mixed. The carbon number of the alkyl group can be, for example, 1 or more, 2 or more, or 4 or more. The carbon number can also be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group.

Preferably, the ring-fused carbazol-9-yl group in which the ring skeleton-constituting carbon atoms are substituted with at least two substituted or unsubstituted aryl groups has a structure represented by the following general formula (2).

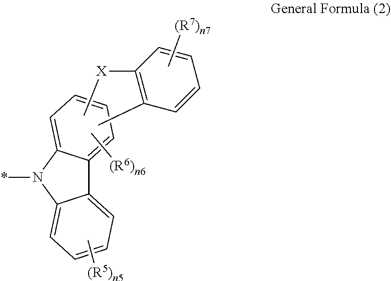

In the general formula (2), X represents O, S, N(R⁸) or C(R⁹)(R¹⁰). Preferably, X is O, S or N(R⁸), and more preferably O or S. In one preferred aspect of the present invention, X is O. In one preferred aspect of the present invention, X is S. In the general formula (2), the upper right benzene ring to which (R⁷)ₙ₇ bonds and the central benzene ring to which (R⁶)ₙ₆ bonds are linked by a bond through X and a single bond, but the positional relationship between these two bonds is not limited. In the above general formula (2), the bond through X is described above and the single bond is described below, but the general formula (2) also includes a structure where the bond through X is positioned below and the single bond is positioned above.

In the general formula (2), n5 and n7 are each independently an integer of 0 to 4, preferably 0 to 2, and more preferably 0 or 1. n6 is an integer of 0 to 2, and preferably 0 or 1. n5+n6+n7 is an integer of 2 to 10, preferably 2 to 6, more preferably 2 to 4, and further preferably 2 or 3. In one preferred aspect of the present invention, n5 and n7 each are 1, and n6 is 0. In one aspect of the present invention, n5 and n6 each are 1, and n7 is 0. In one aspect of the present invention, n5 and no and n7 each are 1. In one aspect of the present invention, n5 is 2, and n6 and n7 each are 0. In one aspect of the present invention, n7 is 2, and n5 and n6 each are 0.

In the general formula (2), R⁵ to R⁷ each independently represent a substituent (including a deuterium atom). The substituent can be selected from Substituent Group A, can be selected from Substituent Group B, can be selected from Substituent Group C, can be selected from Substituent Group D, or can be selected from Substituent Group E. At least two of R⁵ to R⁷ in the molecule are substituted or unsubstituted aryl groups. Regarding the description and the preferred range of the substituted or unsubstituted aryl group as referred to herein, reference can be made to the description of the above-mentioned “aryl group”, and the description of the substituted or unsubstituted aryl group with which the ring-fused carbazol-9-yl group can be substituted.

In the general formula (2), R⁵'s bonding to the neighboring ring skeleton-constituting carbon atoms, R⁶'s bonding to the neighboring ring skeleton-constituting carbon atoms, and R⁷'s bonding to the neighboring ring skeleton-constituting carbon atoms each can bond to each other to form a cyclic structure. Regarding the cyclic structure to be formed, reference can be made to the description of the ring fusing to the carbazol-9-yl group described above. In one aspect of the present invention, R⁵'s bonding to the neighboring ring skeleton-constituting carbon atoms bond to each other to form a cyclic structure, preferably a benzofuro structure or a benzothieno structure. In one aspect of the present invention, R⁷'s bonding to the neighboring ring skeleton-constituting carbon atoms bond to each other to form a cyclic structure, preferably a benzofuro structure or a benzothieno structure. In one aspect of the present invention, R⁵'s, R⁶'s and R⁷'s each do not bond to each other to form a cyclic structure. R⁵ and R⁶, and R⁶ and R⁷ each do not bond to each other to form a cyclic structure.

* in the general formula (2) indicates the bonding site to the benzene ring in the general formula (1).

In one preferred aspect of the present invention, X is O, n5 and n7 each are 1, n6 is 0 or 1, and R⁵, R⁶ and R⁷, or R⁵ and R⁷ each are a substituted or unsubstituted aryl group. In one preferred aspect of the present invention, X is S, n5 and n7 each are 1, n6 is 0 or 1, and R⁵, R⁶ and R⁷, or R⁵ and R⁷ each are a substituted or unsubstituted aryl group.

Specific examples of the ring-fused carbazol-9-yl group in which the ring skeleton-constituting carbon atoms are substituted with at least two substituted or unsubstituted aryl groups and which can be employed as R¹ and 0 or 1 of R² to R⁴ in the general formula (1) are shown below. However, the ring-fused carbazol-9-yl group in which the ring skeleton-constituting carbon atoms are substituted with at least two substituted or unsubstituted aryl groups and which can be employed in the present invention shall not be construed as being limited by the following specific examples. In the following specific examples, * indicates a bonding site, and Ph represents a phenyl group. A methyl group is not shown. Accordingly, for example, D181 to D240 have a methyl group.

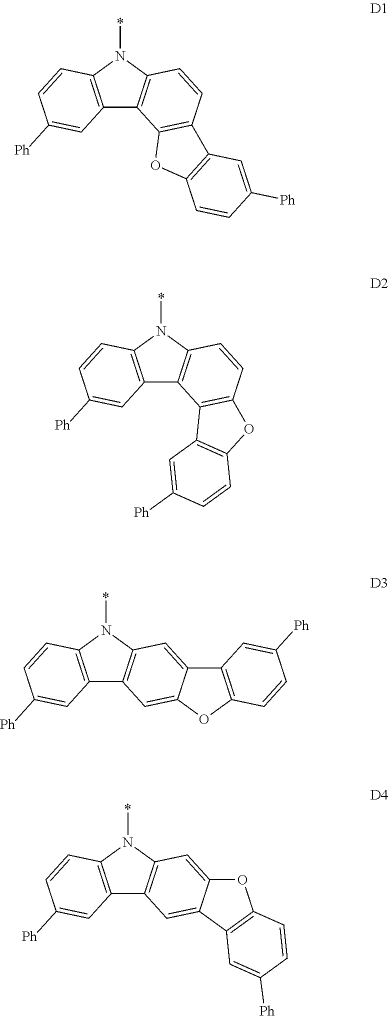 [SMILES: *n1c2ccc(-c3ccccc3)cc2c2c3oc4ccc(-c5ccccc5)cc4c3ccc21 ; *n1c2ccc(-c3ccccc3)cc2c2c3c(ccc21)oc1ccc(-c2ccccc2)cc13 ; *n1c2ccc(-c3ccccc3)cc2c2cc3oc4ccc(-c5ccccc5)cc4c3cc21 ; *n1c2ccc(-c3ccccc3)cc2c2cc3c(cc21)oc1ccc(-c2ccccc2)cc13]

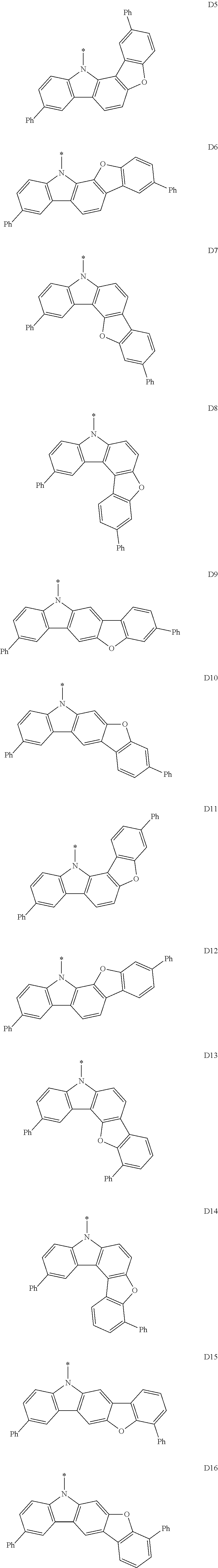

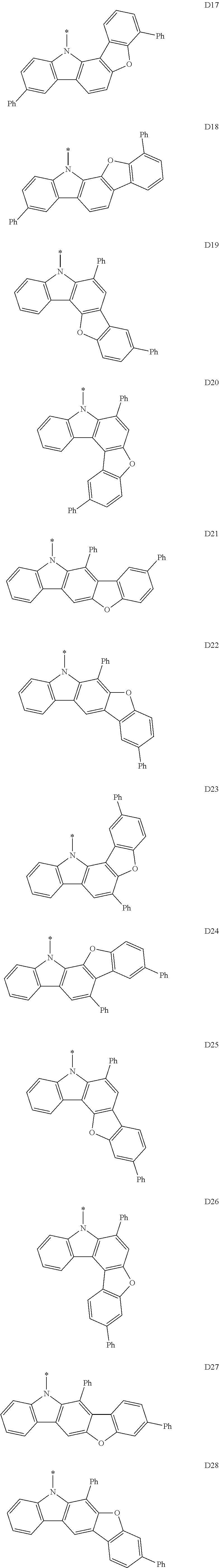

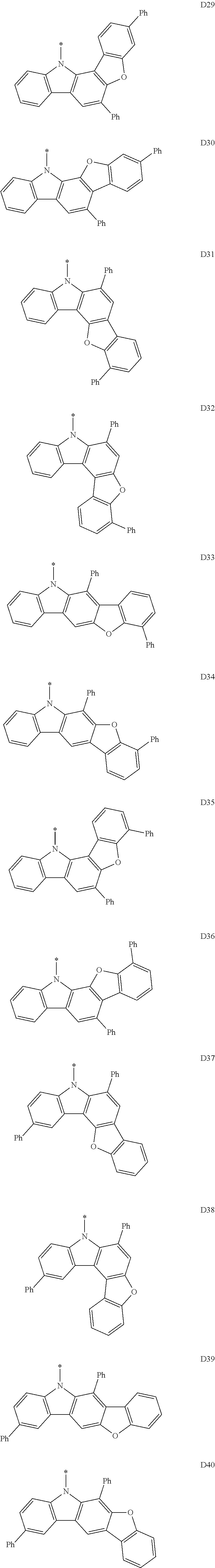

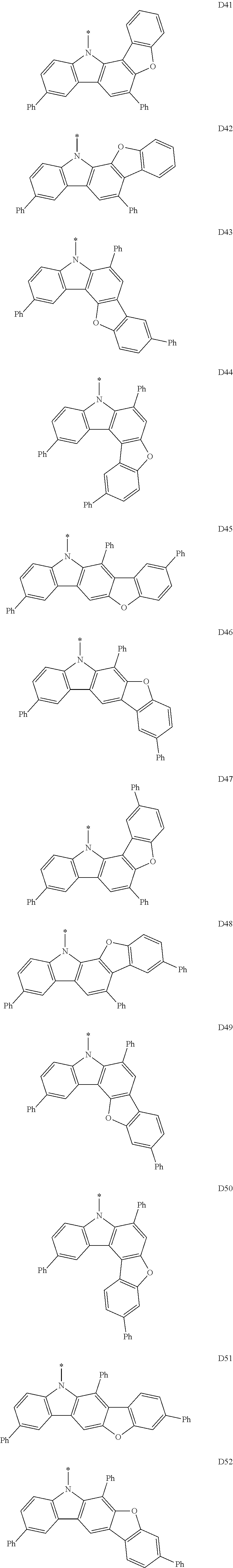

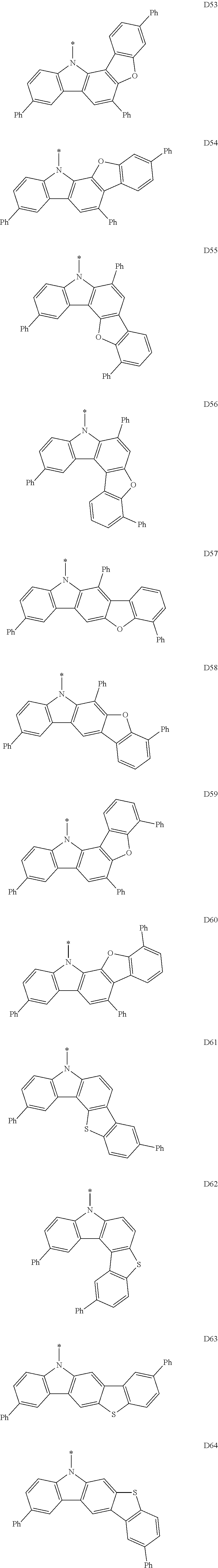

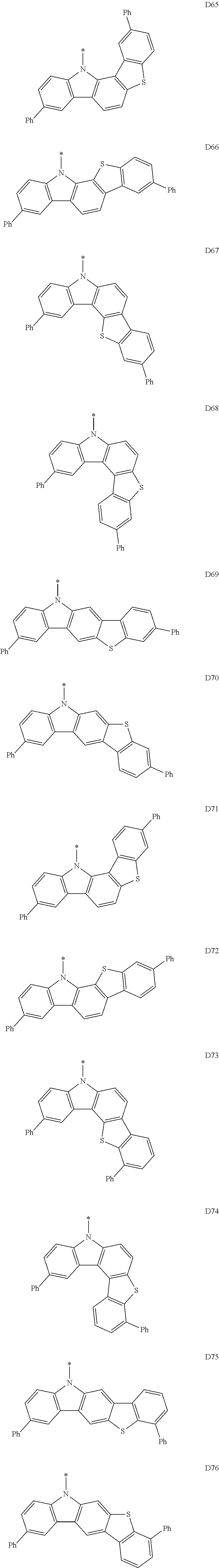

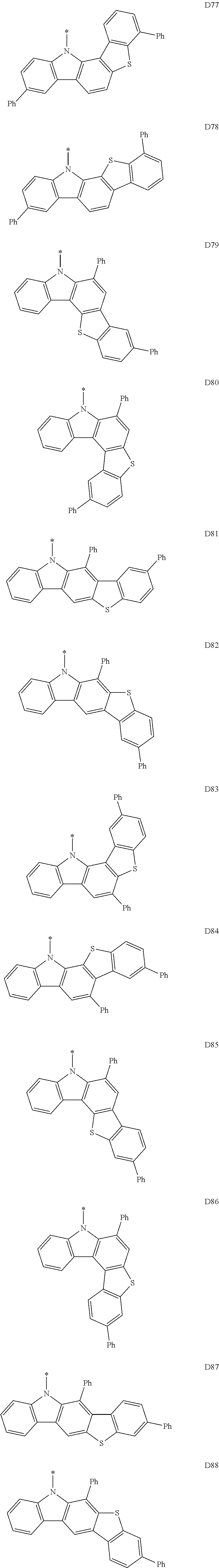

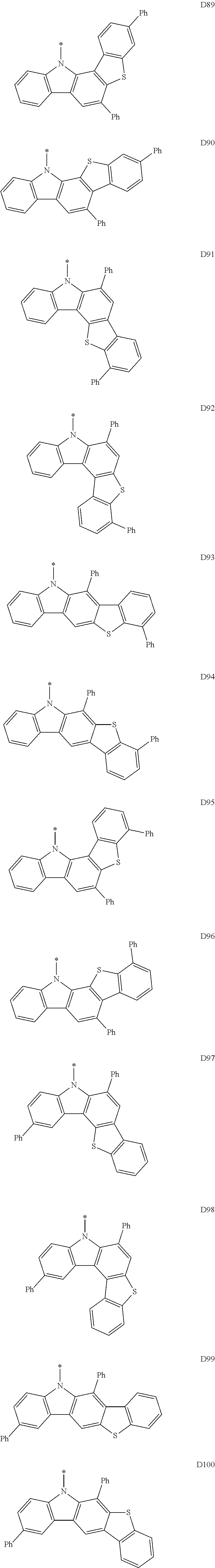

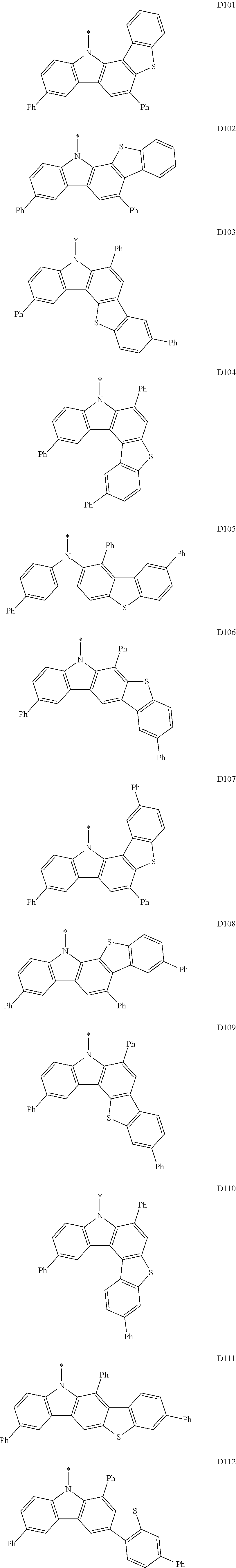

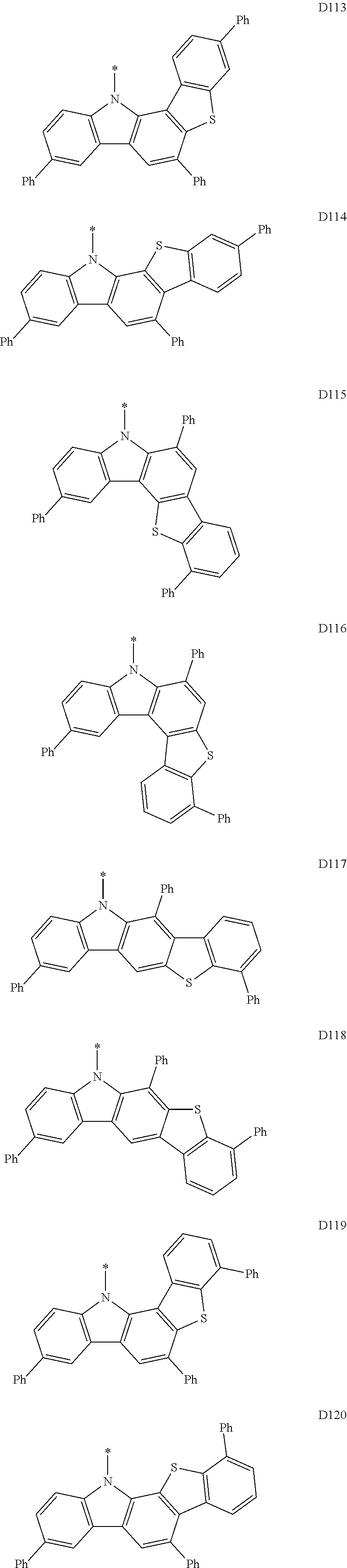

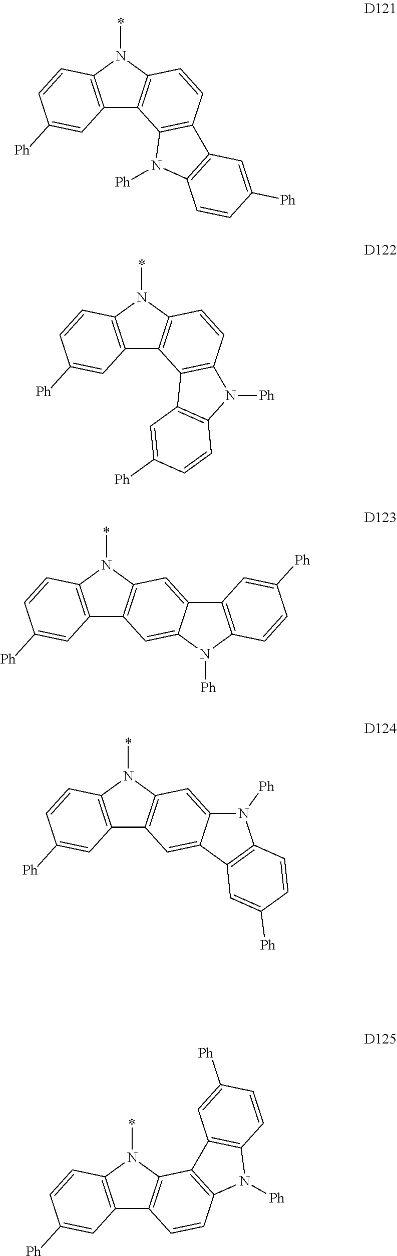 [SMILES: *n1c2ccc(-c3ccccc3)cc2c2c1ccc1c3cc(-c4ccccc4)ccc3n(-c3ccccc3)c12 ; *n1c2ccc(-c3ccccc3)cc2c2c3c4cc(-c5ccccc5)ccc4n(-c4ccccc4)c3ccc21 ; *n1c2ccc(-c3ccccc3)cc2c2cc3c(cc21)c1cc(-c2ccccc2)ccc1n3-c1ccccc1 ; *n1c2ccc(-c3ccccc3)cc2c2cc3c4cc(-c5ccccc5)ccc4n(-c4ccccc4)c3cc21 ; *n1c2ccc(-c3ccccc3)cc2c2ccc3c(c4cc(-c5ccccc5)ccc4n3-c3ccccc3)c21]

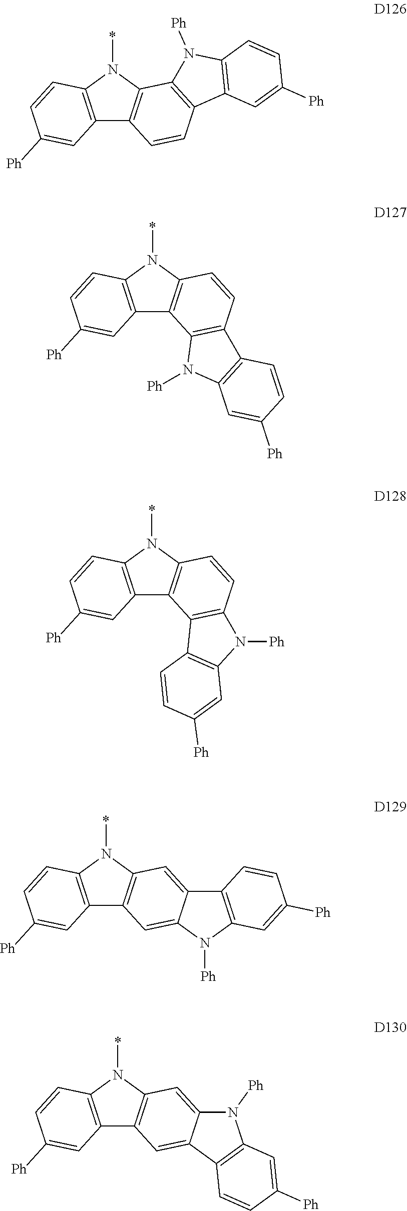 [SMILES: *n1c2ccc(-c3ccccc3)cc2c2ccc3c4cc(-c5ccccc5)ccc4n(-c4ccccc4)c3c21 ; *n1c2ccc(-c3ccccc3)cc2c2c1ccc1c3ccc(-c4ccccc4)cc3n(-c3ccccc3)c12 ; *n1c2ccc(-c3ccccc3)cc2c2c3c4ccc(-c5ccccc5)cc4n(-c4ccccc4)c3ccc21 ; *n1c2ccc(-c3ccccc3)cc2c2cc3c(cc21)c1ccc(-c2ccccc2)cc1n3-c1ccccc1 ; *n1c2ccc(-c3ccccc3)cc2c2cc3c4ccc(-c5ccccc5)cc4n(-c4ccccc4)c3cc21]

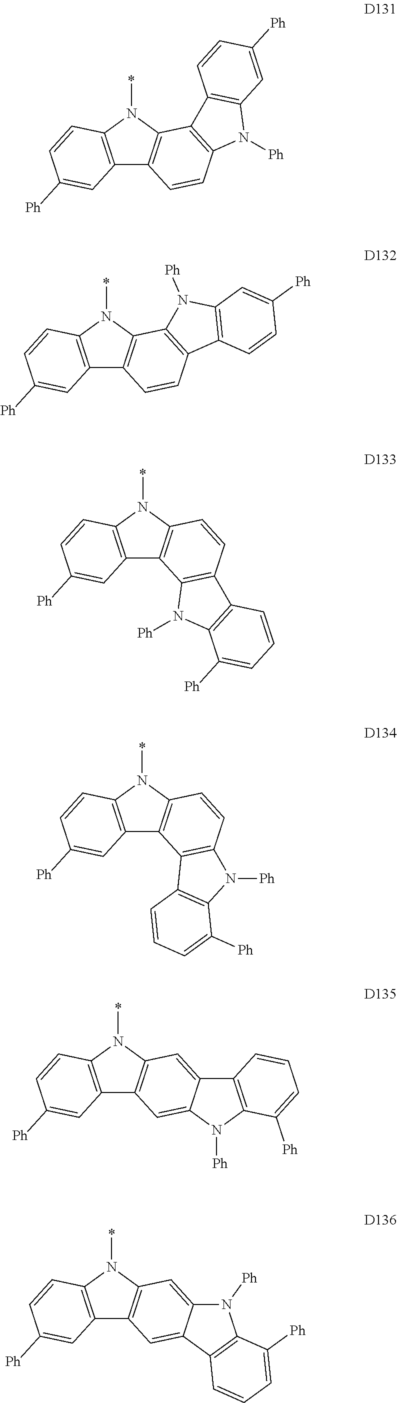 [SMILES: *n1c2ccc(-c3ccccc3)cc2c2ccc3c(c4ccc(-c5ccccc5)cc4n3-c3ccccc3)c21 ; *n1c2ccc(-c3ccccc3)cc2c2ccc3c4ccc(-c5ccccc5)cc4n(-c4ccccc4)c3c21 ; *n1c2ccc(-c3ccccc3)cc2c2c1ccc1c3cccc(-c4ccccc4)c3n(-c3ccccc3)c12 ; *n1c2ccc(-c3ccccc3)cc2c2c3c4cccc(-c5ccccc5)c4n(-c4ccccc4)c3ccc21 ; *n1c2ccc(-c3ccccc3)cc2c2cc3c(cc21)c1cccc(-c2ccccc2)c1n3-c1ccccc1 ; *n1c2ccc(-c3ccccc3)cc2c2cc3c4cccc(-c5ccccc5)c4n(-c4ccccc4)c3cc21]

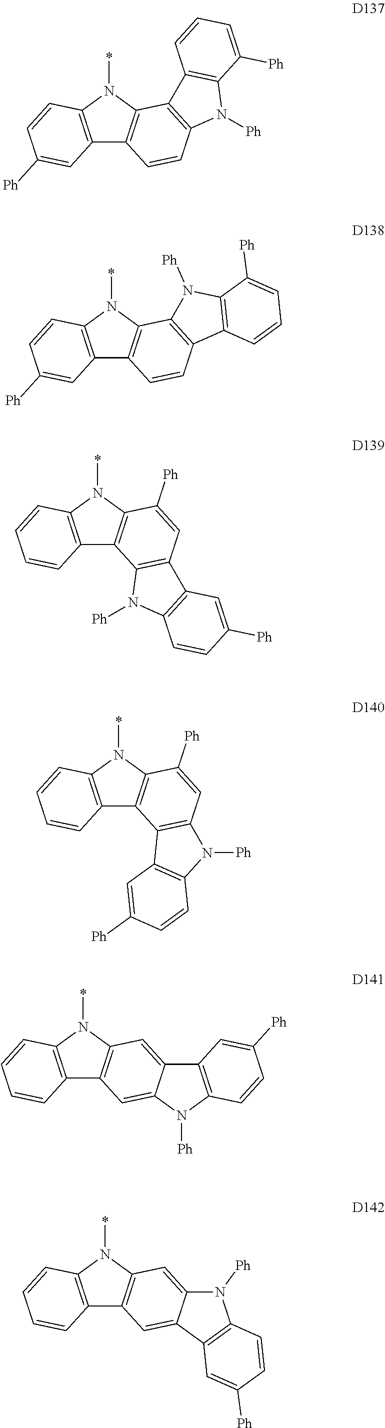 [SMILES: *n1c2ccc(-c3ccccc3)cc2c2ccc3c(c4cccc(-c5ccccc5)c4n3-c3ccccc3)c21 ; *n1c2ccc(-c3ccccc3)cc2c2ccc3c4cccc(-c5ccccc5)c4n(-c4ccccc4)c3c21 ; *n1c2ccccc2c2c1c(-c1ccccc1)cc1c3cc(-c4ccccc4)ccc3n(-c3ccccc3)c12 ; *n1c2ccccc2c2c3c4cc(-c5ccccc5)ccc4n(-c4ccccc4)c3cc(-c3ccccc3)c21 ; *n1c2ccccc2c2cc3c(cc21)c1cc(-c2ccccc2)ccc1n3-c1ccccc1 ; *n1c2ccccc2c2cc3c4cc(-c5ccccc5)ccc4n(-c4ccccc4)c3cc21]

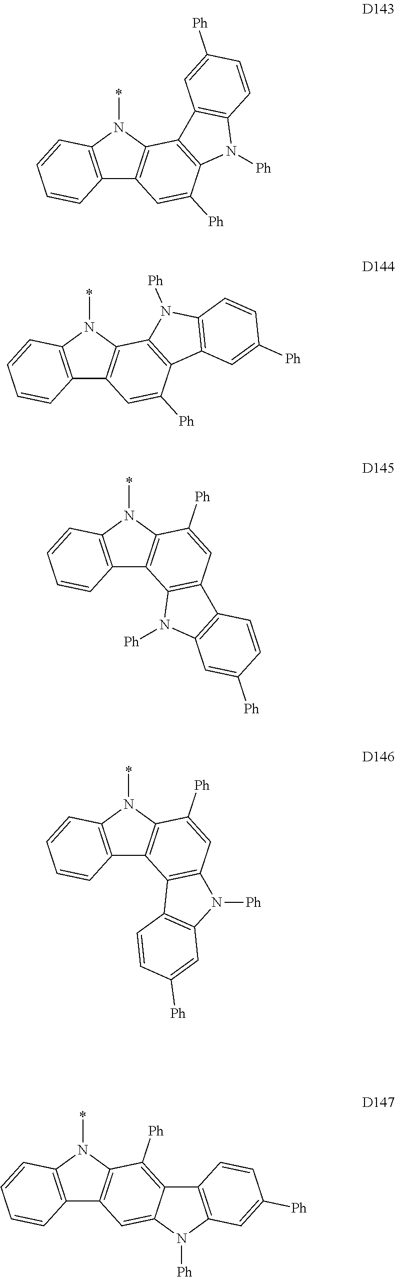 [SMILES: *n1c2ccccc2c2cc(-c3ccccc3)c3c(c4cc(-c5ccccc5)ccc4n3-c3ccccc3)c21 ; *n1c2ccccc2c2cc(-c3ccccc3)c3c4cc(-c5ccccc5)ccc4n(-c4ccccc4)c3c21 ; *n1c2ccccc2c2c1c(-c1ccccc1)cc1c3ccc(-c4ccccc4)cc3n(-c3ccccc3)c12 ; *n1c2ccccc2c2c3c4ccc(-c5ccccc5)cc4n(-c4ccccc4)c3cc(-c3ccccc3)c21 ; *n1c2ccccc2c2cc3c(c(-c4ccccc4)c21)c1ccc(-c2ccccc2)cc1n3-c1ccccc1]

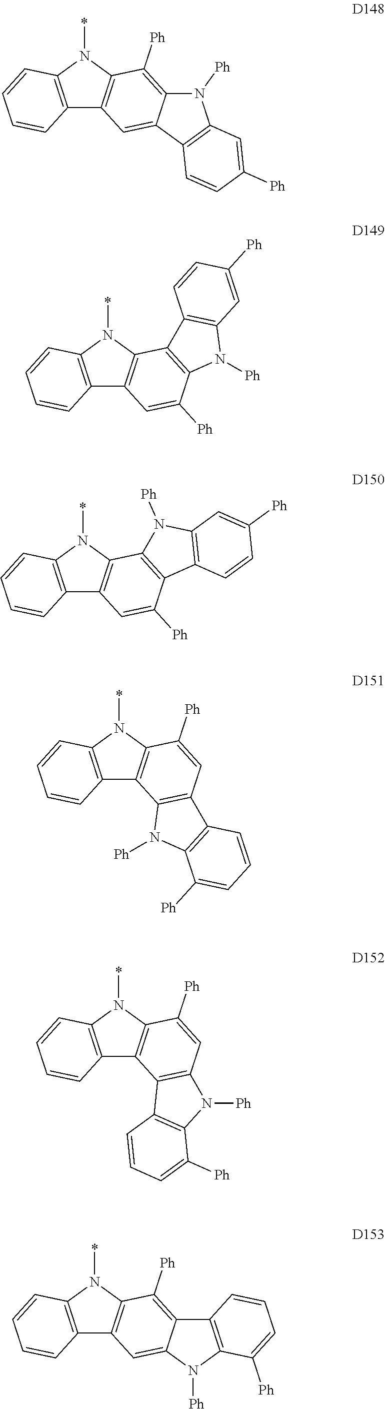 [SMILES: *n1c2ccccc2c2cc3c4ccc(-c5ccccc5)cc4n(-c4ccccc4)c3c(-c3ccccc3)c21 ; *n1c2ccccc2c2cc(-c3ccccc3)c3c(c4ccc(-c5ccccc5)cc4n3-c3ccccc3)c21 ; *n1c2ccccc2c2cc(-c3ccccc3)c3c4ccc(-c5ccccc5)cc4n(-c4ccccc4)c3c21 ; *n1c2ccccc2c2c1c(-c1ccccc1)cc1c3cccc(-c4ccccc4)c3n(-c3ccccc3)c12 ; *n1c2ccccc2c2c3c4cccc(-c5ccccc5)c4n(-c4ccccc4)c3cc(-c3ccccc3)c21 ; *n1c2ccccc2c2cc3c(c(-c4ccccc4)c21)c1cccc(-c2ccccc2)c1n3-c1ccccc1]

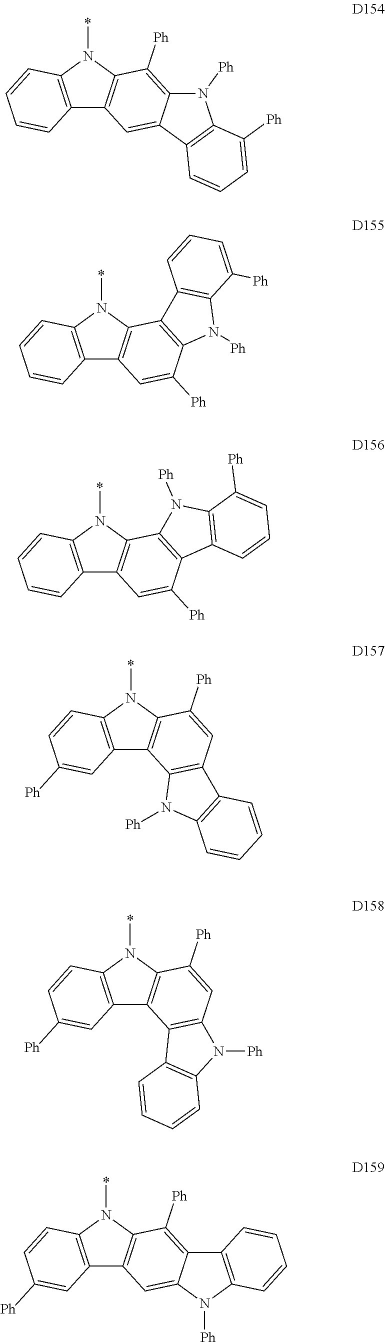 [SMILES: *n1c2ccccc2c2cc3c4cccc(-c5ccccc5)c4n(-c4ccccc4)c3c(-c3ccccc3)c21 ; *n1c2ccccc2c2cc(-c3ccccc3)c3c(c4cccc(-c5ccccc5)c4n3-c3ccccc3)c21 ; *n1c2ccccc2c2cc(-c3ccccc3)c3c4cccc(-c5ccccc5)c4n(-c4ccccc4)c3c21 ; *n1c2ccc(-c3ccccc3)cc2c2c1c(-c1ccccc1)cc1c3ccccc3n(-c3ccccc3)c12 ; *n1c2ccc(-c3ccccc3)cc2c2c3c4ccccc4n(-c4ccccc4)c3cc(-c3ccccc3)c21 ; *n1c2ccc(-c3ccccc3)cc2c2cc3c(c(-c4ccccc4)c21)c1ccccc1n3-c1ccccc1]

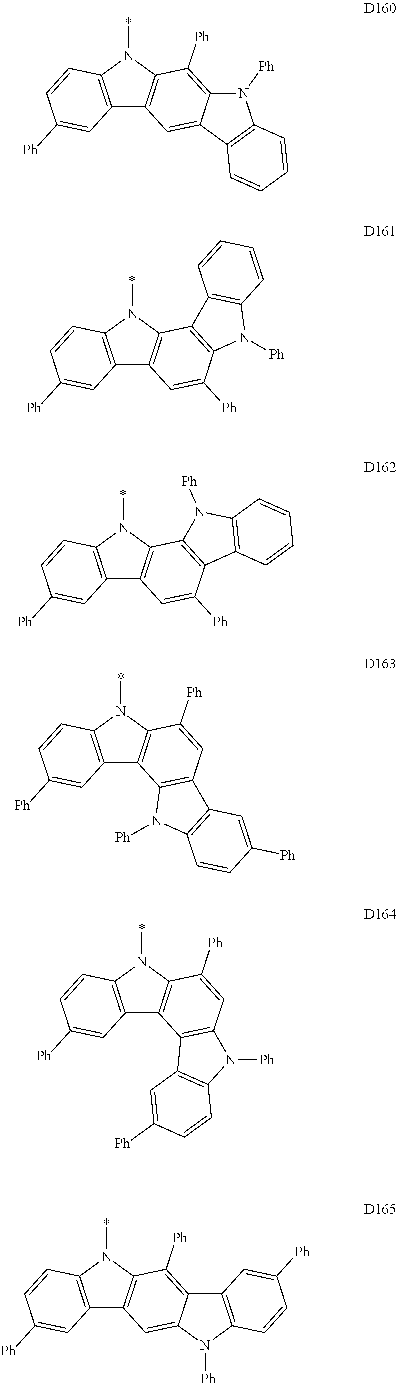 [SMILES: *n1c2ccc(-c3ccccc3)cc2c2cc3c4ccccc4n(-c4ccccc4)c3c(-c3ccccc3)c21 ; *n1c2ccc(-c3ccccc3)cc2c2cc(-c3ccccc3)c3c(c4ccccc4n3-c3ccccc3)c21 ; *n1c2ccc(-c3ccccc3)cc2c2cc(-c3ccccc3)c3c4ccccc4n(-c4ccccc4)c3c21 ; *n1c2ccc(-c3ccccc3)cc2c2c1c(-c1ccccc1)cc1c3cc(-c4ccccc4)ccc3n(-c3ccccc3)c12 ; *n1c2ccc(-c3ccccc3)cc2c2c3c4cc(-c5ccccc5)ccc4n(-c4ccccc4)c3cc(-c3ccccc3)c21 ; *n1c2ccc(-c3ccccc3)cc2c2cc3c(c(-c4ccccc4)c21)c1cc(-c2ccccc2)ccc1n3-c1ccccc1]

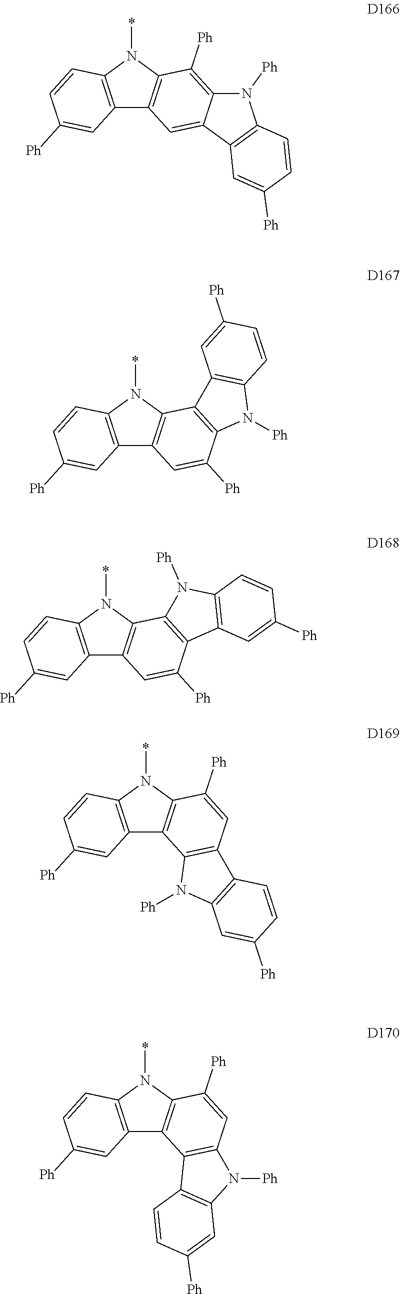 [SMILES: *n1c2ccc(-c3ccccc3)cc2c2cc3c4cc(-c5ccccc5)ccc4n(-c4ccccc4)c3c(-c3ccccc3)c21 ; *n1c2ccc(-c3ccccc3)cc2c2cc(-c3ccccc3)c3c(c4cc(-c5ccccc5)ccc4n3-c3ccccc3)c21 ; *n1c2ccc(-c3ccccc3)cc2c2cc(-c3ccccc3)c3c4cc(-c5ccccc5)ccc4n(-c4ccccc4)c3c21 ; *n1c2ccc(-c3ccccc3)cc2c2c1c(-c1ccccc1)cc1c3ccc(-c4ccccc4)cc3n(-c3ccccc3)c12 ; *n1c2ccc(-c3ccccc3)cc2c2c3c4ccc(-c5ccccc5)cc4n(-c4ccccc4)c3cc(-c3ccccc3)c21]

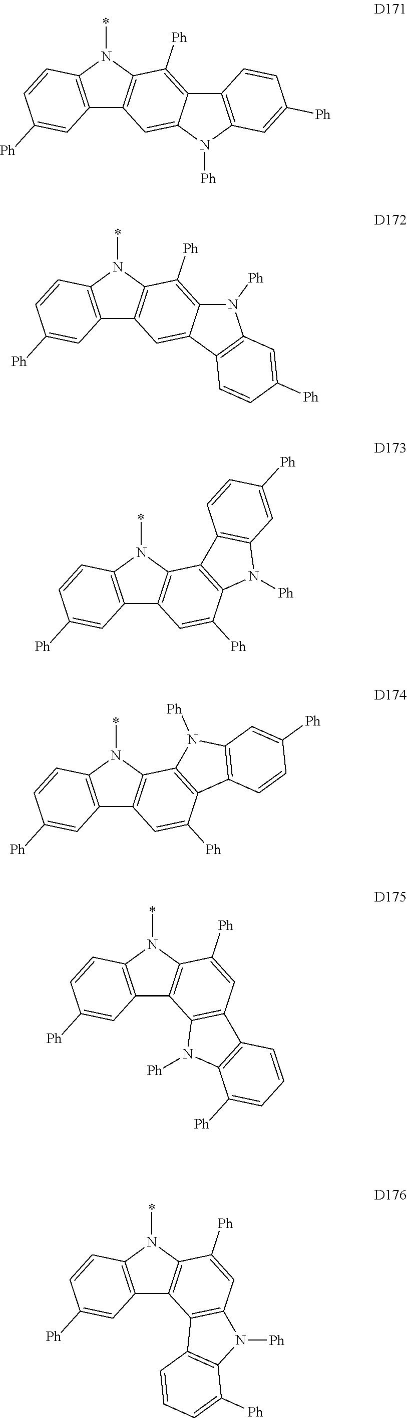 [SMILES: *n1c2ccc(-c3ccccc3)cc2c2cc3c(c(-c4ccccc4)c21)c1ccc(-c2ccccc2)cc1n3-c1ccccc1 ; *n1c2ccc(-c3ccccc3)cc2c2cc3c4ccc(-c5ccccc5)cc4n(-c4ccccc4)c3c(-c3ccccc3)c21 ; *n1c2ccc(-c3ccccc3)cc2c2cc(-c3ccccc3)c3c(c4ccc(-c5ccccc5)cc4n3-c3ccccc3)c21 ; *n1c2ccc(-c3ccccc3)cc2c2cc(-c3ccccc3)c3c4ccc(-c5ccccc5)cc4n(-c4ccccc4)c3c21 ; *n1c2ccc(-c3ccccc3)cc2c2c1c(-c1ccccc1)cc1c3cccc(-c4ccccc4)c3n(-c3ccccc3)c12 ; *n1c2ccc(-c3ccccc3)cc2c2c3c4cccc(-c5ccccc5)c4n(-c4ccccc4)c3cc(-c3ccccc3)c21]

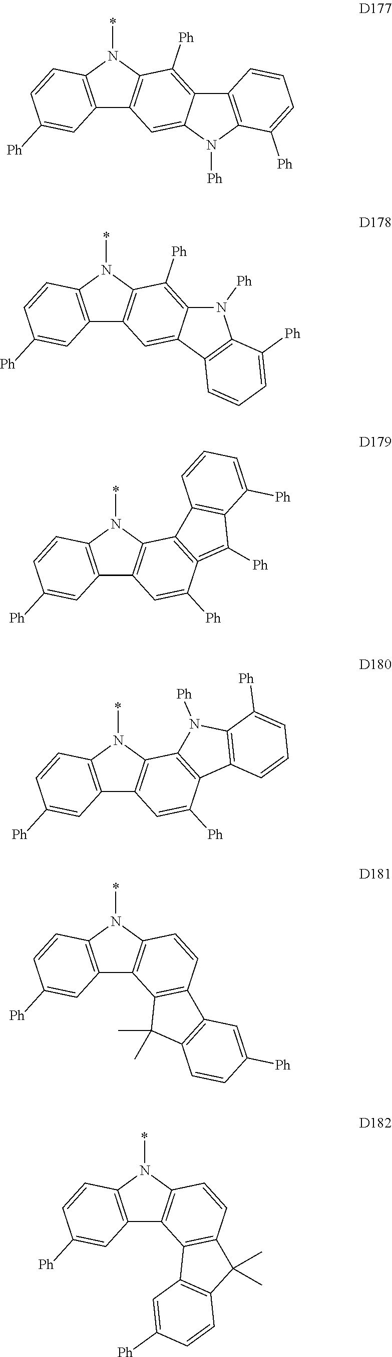

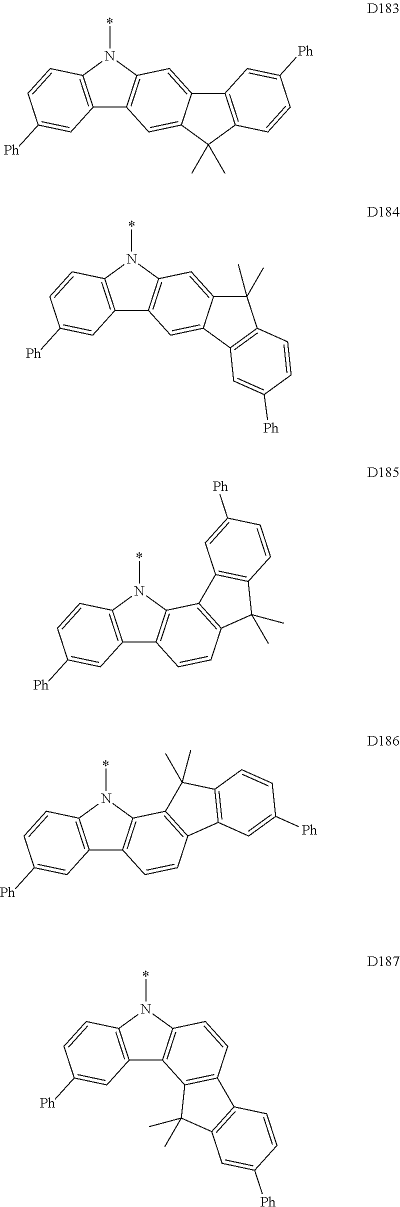 [SMILES: *n1c2ccc(-c3ccccc3)cc2c2cc3c(cc21)-c1cc(-c2ccccc2)ccc1C3(C)C ; *n1c2ccc(-c3ccccc3)cc2c2cc3c(cc21)C(C)(C)c1ccc(-c2ccccc2)cc1-3 ; *n1c2ccc(-c3ccccc3)cc2c2ccc3c(c21)-c1cc(-c2ccccc2)ccc1C3(C)C ; *n1c2ccc(-c3ccccc3)cc2c2ccc3c(c21)C(C)(C)c1ccc(-c2ccccc2)cc1-3 ; *n1c2ccc(-c3ccccc3)cc2c2c3c(ccc21)-c1ccc(-c2ccccc2)cc1C3(C)C]

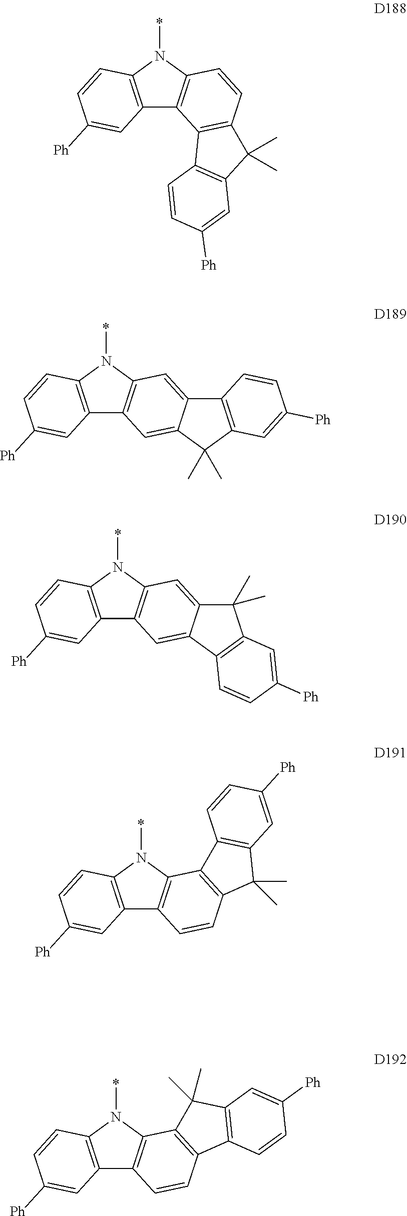 [SMILES: *n1c2ccc(-c3ccccc3)cc2c2c3c(ccc21)C(C)(C)c1cc(-c2ccccc2)ccc1-3 ; *n1c2ccc(-c3ccccc3)cc2c2cc3c(cc21)-c1ccc(-c2ccccc2)cc1C3(C)C ; *n1c2ccc(-c3ccccc3)cc2c2cc3c(cc21)C(C)(C)c1cc(-c2ccccc2)ccc1-3 ; *n1c2ccc(-c3ccccc3)cc2c2ccc3c(c21)-c1ccc(-c2ccccc2)cc1C3(C)C ; *n1c2ccc(-c3ccccc3)cc2c2ccc3c(c21)C(C)(C)c1cc(-c2ccccc2)ccc1-3]

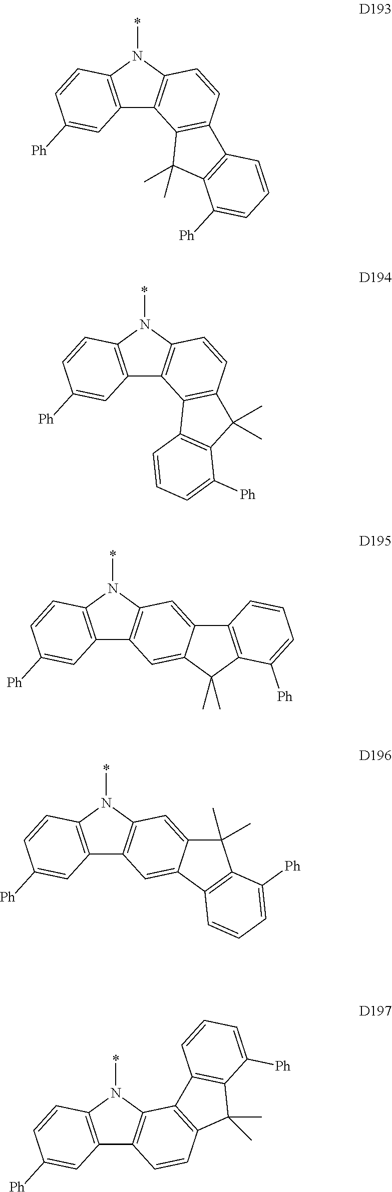 [SMILES: *n1c2ccc(-c3ccccc3)cc2c2c3c(ccc21)-c1cccc(-c2ccccc2)c1C3(C)C ; *n1c2ccc(-c3ccccc3)cc2c2c3c(ccc21)C(C)(C)c1c(-c2ccccc2)cccc1-3 ; *n1c2ccc(-c3ccccc3)cc2c2cc3c(cc21)-c1cccc(-c2ccccc2)c1C3(C)C ; *n1c2ccc(-c3ccccc3)cc2c2cc3c(cc21)C(C)(C)c1c(-c2ccccc2)cccc1-3 ; *n1c2ccc(-c3ccccc3)cc2c2ccc3c(c21)-c1cccc(-c2ccccc2)c1C3(C)C]

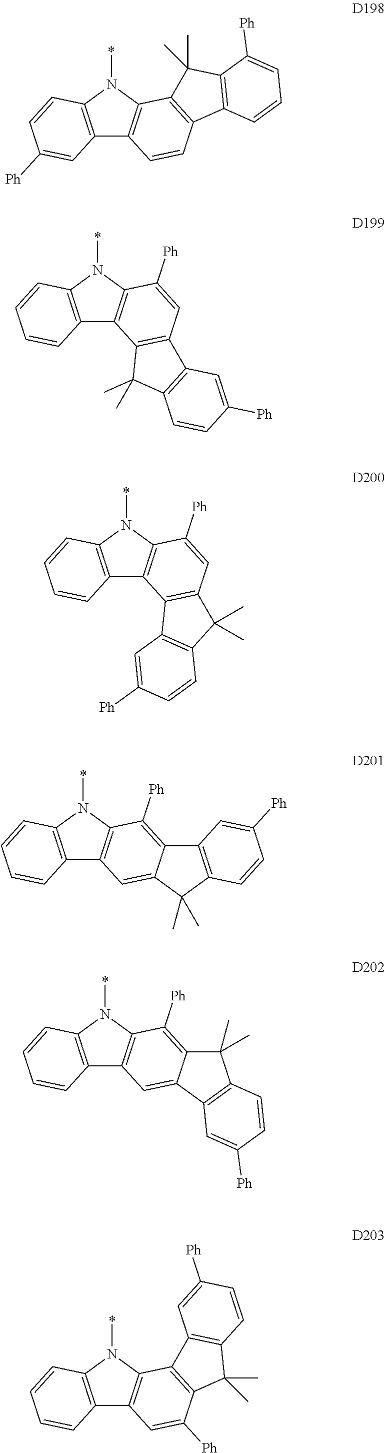 [SMILES: *n1c2ccc(-c3ccccc3)cc2c2ccc3c(c21)C(C)(C)c1c(-c2ccccc2)cccc1-3 ; *n1c2ccccc2c2c3c(cc(-c4ccccc4)c21)-c1cc(-c2ccccc2)ccc1C3(C)C ; *n1c2ccccc2c2c3c(cc(-c4ccccc4)c21)C(C)(C)c1ccc(-c2ccccc2)cc1-3 ; *n1c2ccccc2c2cc3c(c(-c4ccccc4)c21)-c1cc(-c2ccccc2)ccc1C3(C)C ; *n1c2ccccc2c2cc3c(c(-c4ccccc4)c21)C(C)(C)c1ccc(-c2ccccc2)cc1-3 ; *n1c2ccccc2c2cc(-c3ccccc3)c3c(c21)-c1cc(-c2ccccc2)ccc1C3(C)C]

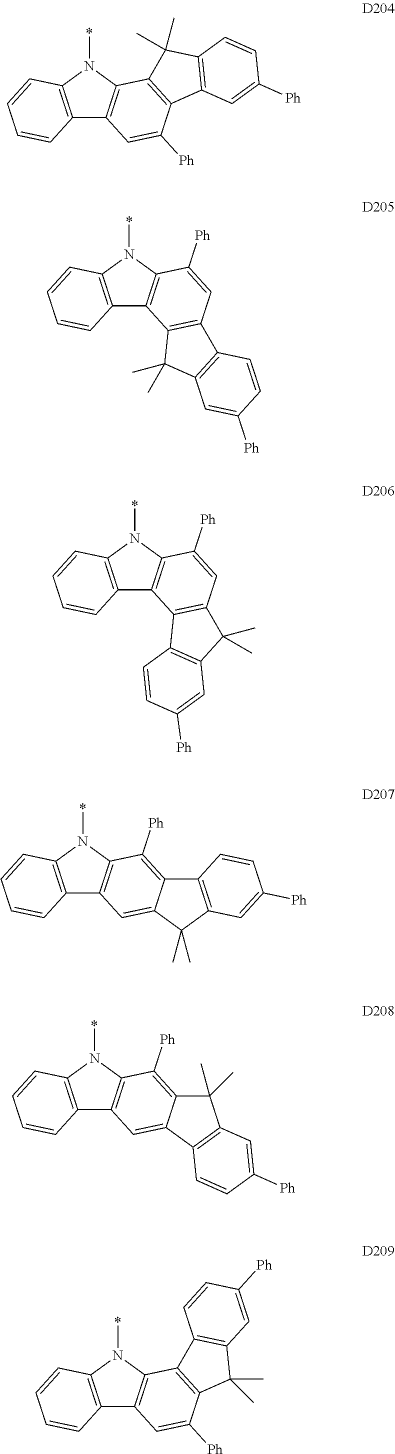 [SMILES: *n1c2ccccc2c2cc(-c3ccccc3)c3c(c21)C(C)(C)c1ccc(-c2ccccc2)cc1-3 ; *n1c2ccccc2c2c3c(cc(-c4ccccc4)c21)-c1ccc(-c2ccccc2)cc1C3(C)C ; *n1c2ccccc2c2c3c(cc(-c4ccccc4)c21)C(C)(C)c1cc(-c2ccccc2)ccc1-3 ; *n1c2ccccc2c2cc3c(c(-c4ccccc4)c21)-c1ccc(-c2ccccc2)cc1C3(C)C ; *n1c2ccccc2c2cc3c(c(-c4ccccc4)c21)C(C)(C)c1cc(-c2ccccc2)ccc1-3 ; *n1c2ccccc2c2cc(-c3ccccc3)c3c(c21)-c1ccc(-c2ccccc2)cc1C3(C)C]

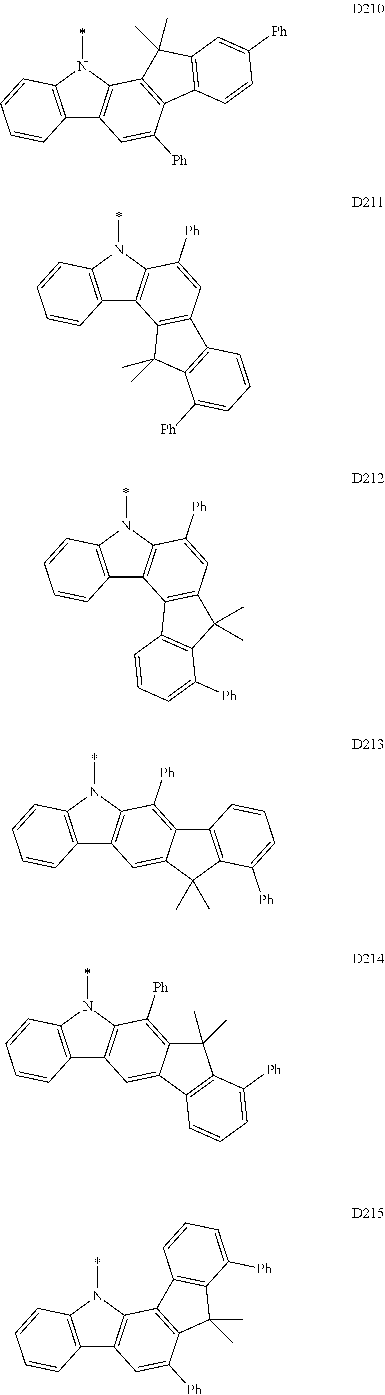 [SMILES: *n1c2ccccc2c2cc(-c3ccccc3)c3c(c21)C(C)(C)c1cc(-c2ccccc2)ccc1-3 ; *n1c2ccccc2c2c3c(cc(-c4ccccc4)c21)-c1cccc(-c2ccccc2)c1C3(C)C ; *n1c2ccccc2c2c3c(cc(-c4ccccc4)c21)C(C)(C)c1c(-c2ccccc2)cccc1-3 ; *n1c2ccccc2c2cc3c(c(-c4ccccc4)c21)-c1cccc(-c2ccccc2)c1C3(C)C ; *n1c2ccccc2c2cc3c(c(-c4ccccc4)c21)C(C)(C)c1c(-c2ccccc2)cccc1-3 ; *n1c2ccccc2c2cc(-c3ccccc3)c3c(c21)-c1cccc(-c2ccccc2)c1C3(C)C]

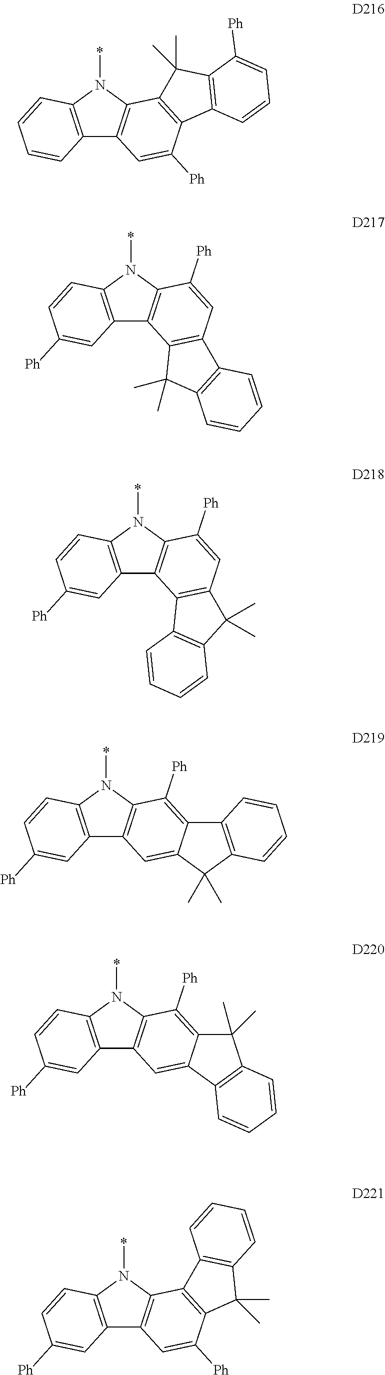 [SMILES: *n1c2ccccc2c2cc(-c3ccccc3)c3c(c21)C(C)(C)c1c(-c2ccccc2)cccc1-3 ; *n1c2ccc(-c3ccccc3)cc2c2c3c(cc(-c4ccccc4)c21)-c1ccccc1C3(C)C ; *n1c2ccc(-c3ccccc3)cc2c2c3c(cc(-c4ccccc4)c21)C(C)(C)c1ccccc1-3 ; *n1c2ccc(-c3ccccc3)cc2c2cc3c(c(-c4ccccc4)c21)-c1ccccc1C3(C)C ; *n1c2ccc(-c3ccccc3)cc2c2cc3c(c(-c4ccccc4)c21)C(C)(C)c1ccccc1-3 ; *n1c2ccc(-c3ccccc3)cc2c2cc(-c3ccccc3)c3c(c21)-c1ccccc1C3(C)C]

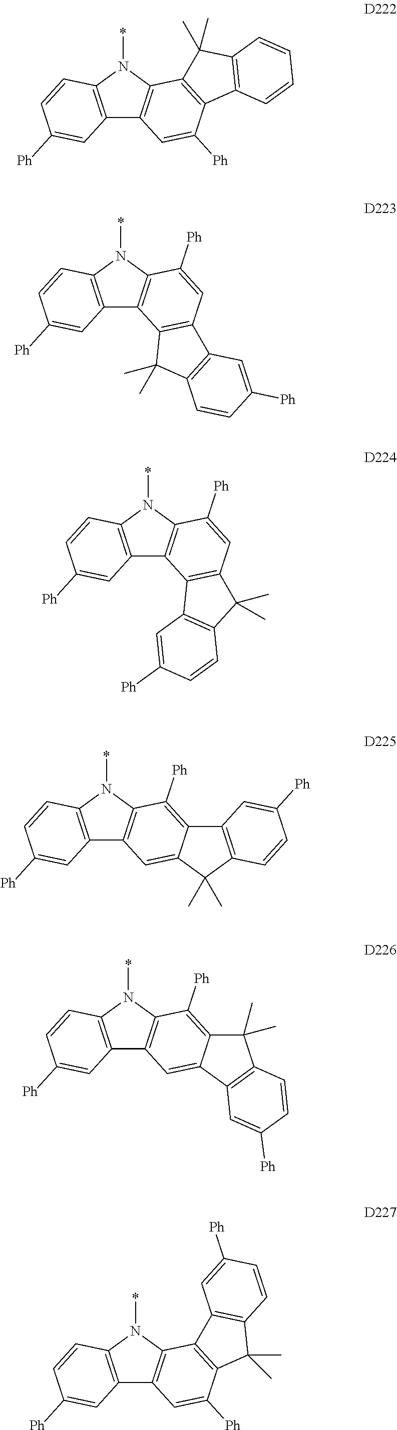 [SMILES: *n1c2ccc(-c3ccccc3)cc2c2cc(-c3ccccc3)c3c(c21)C(C)(C)c1ccccc1-3 ; *n1c2ccc(-c3ccccc3)cc2c2c3c(cc(-c4ccccc4)c21)-c1cc(-c2ccccc2)ccc1C3(C)C ; *n1c2ccc(-c3ccccc3)cc2c2c3c(cc(-c4ccccc4)c21)C(C)(C)c1ccc(-c2ccccc2)cc1-3 ; *n1c2ccc(-c3ccccc3)cc2c2cc3c(c(-c4ccccc4)c21)-c1cc(-c2ccccc2)ccc1C3(C)C ; *n1c2ccc(-c3ccccc3)cc2c2cc3c(c(-c4ccccc4)c21)C(C)(C)c1ccc(-c2ccccc2)cc1-3 ; *n1c2ccc(-c3ccccc3)cc2c2cc(-c3ccccc3)c3c(c21)-c1cc(-c2ccccc2)ccc1C3(C)C]

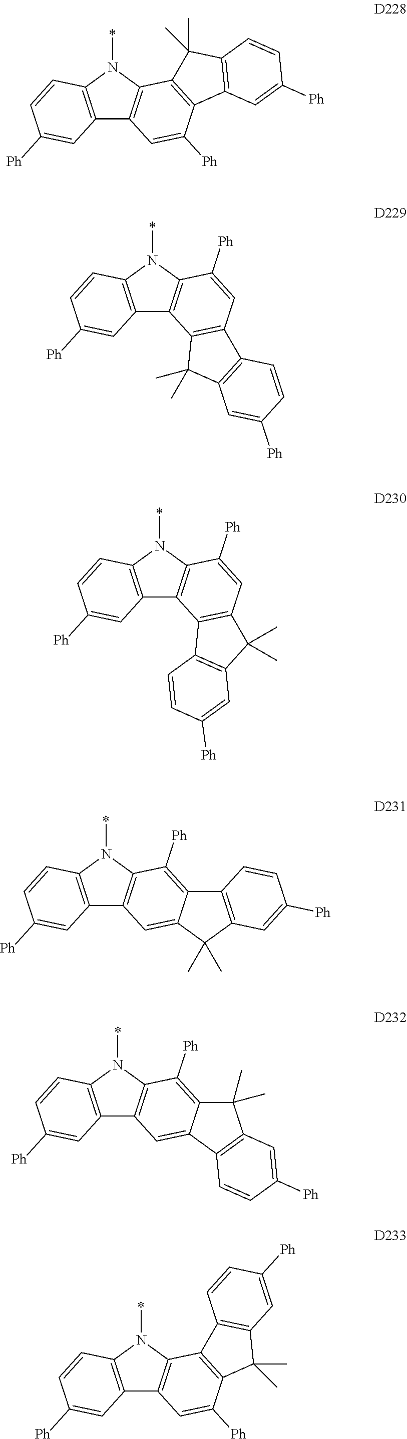 [SMILES: *n1c2ccc(-c3ccccc3)cc2c2cc(-c3ccccc3)c3c(c21)C(C)(C)c1ccc(-c2ccccc2)cc1-3 ; *n1c2ccc(-c3ccccc3)cc2c2c3c(cc(-c4ccccc4)c21)-c1ccc(-c2ccccc2)cc1C3(C)C ; *n1c2ccc(-c3ccccc3)cc2c2c3c(cc(-c4ccccc4)c21)C(C)(C)c1cc(-c2ccccc2)ccc1-3 ; *n1c2ccc(-c3ccccc3)cc2c2cc3c(c(-c4ccccc4)c21)-c1ccc(-c2ccccc2)cc1C3(C)C ; *n1c2ccc(-c3ccccc3)cc2c2cc3c(c(-c4ccccc4)c21)C(C)(C)c1cc(-c2ccccc2)ccc1-3 ; *n1c2ccc(-c3ccccc3)cc2c2cc(-c3ccccc3)c3c(c21)-c1ccc(-c2ccccc2)cc1C3(C)C]

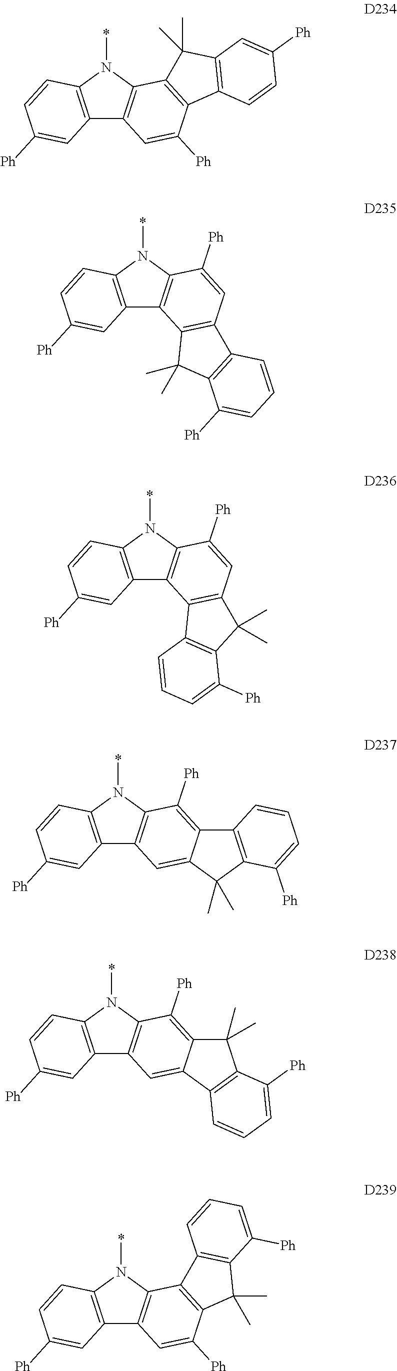 [SMILES: *n1c2ccc(-c3ccccc3)cc2c2cc(-c3ccccc3)c3c(c21)C(C)(C)c1cc(-c2ccccc2)ccc1-3 ; *n1c2ccc(-c3ccccc3)cc2c2c3c(cc(-c4ccccc4)c21)-c1cccc(-c2ccccc2)c1C3(C)C ; *n1c2ccc(-c3ccccc3)cc2c2c3c(cc(-c4ccccc4)c21)C(C)(C)c1c(-c2ccccc2)cccc1-3 ; *n1c2ccc(-c3ccccc3)cc2c2cc3c(c(-c4ccccc4)c21)-c1cccc(-c2ccccc2)c1C3(C)C ; *n1c2ccc(-c3ccccc3)cc2c2cc3c(c(-c4ccccc4)c21)C(C)(C)c1c(-c2ccccc2)cccc1-3 ; *n1c2ccc(-c3ccccc3)cc2c2cc(-c3ccccc3)c3c(c21)-c1cccc(-c2ccccc2)c1C3(C)C]

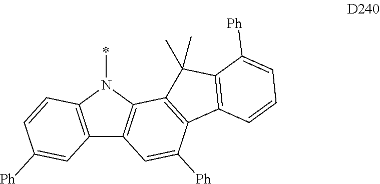 [SMILES: *n1c2ccc(-c3ccccc3)cc2c2cc(-c3ccccc3)c3c(c21)C(C)(C)c1c(-c2ccccc2)cccc1-3]

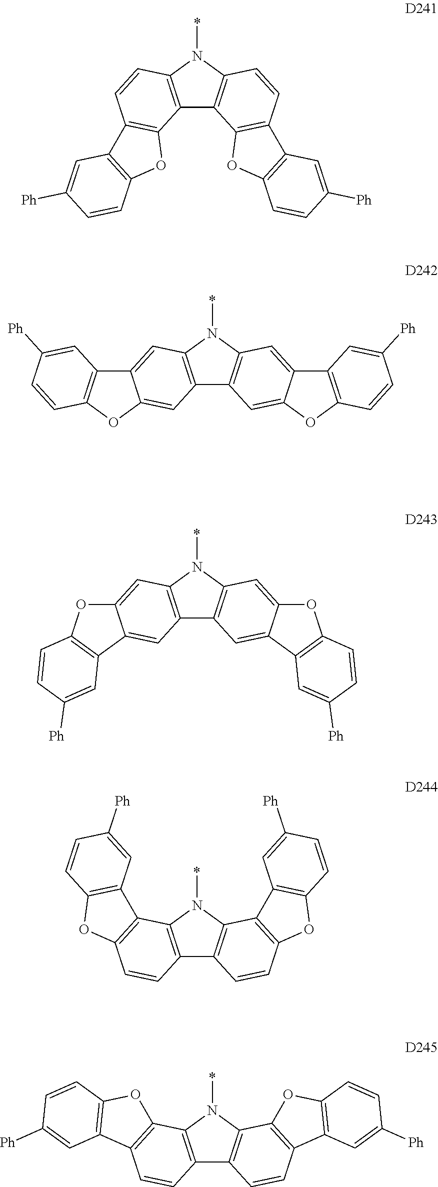 [SMILES: *n1c2ccc3c4cc(-c5ccccc5)ccc4oc3c2c2c3oc4ccc(-c5ccccc5)cc4c3ccc21 ; *n1c2cc3c(cc2c2cc4oc5ccc(-c6ccccc6)cc5c4cc21)oc1ccc(-c2ccccc2)cc13 ; *n1c2cc3oc4ccc(-c5ccccc5)cc4c3cc2c2cc3c(cc21)oc1ccc(-c2ccccc2)cc13 ; *n1c2c(ccc3oc4ccc(-c5ccccc5)cc4c32)c2ccc3oc4ccc(-c5ccccc5)cc4c3c21 ; *n1c2c(ccc3c4cc(-c5ccccc5)ccc4oc32)c2ccc3c4cc(-c5ccccc5)ccc4oc3c21]

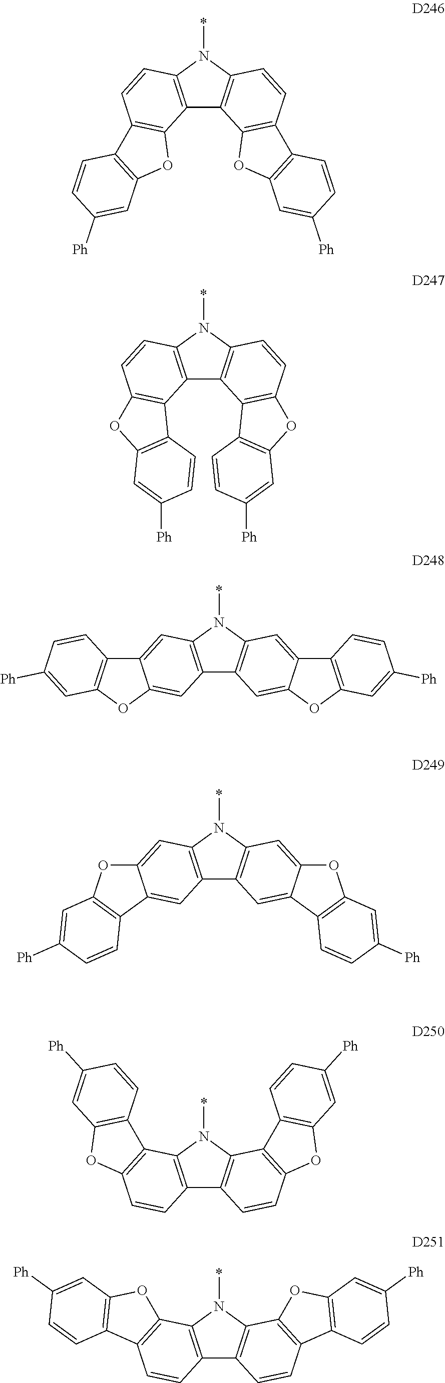 [SMILES: *n1c2ccc3c4ccc(-c5ccccc5)cc4oc3c2c2c3oc4cc(-c5ccccc5)ccc4c3ccc21 ; *n1c2ccc3oc4cc(-c5ccccc5)ccc4c3c2c2c3c(ccc21)oc1cc(-c2ccccc2)ccc13 ; *n1c2cc3c(cc2c2cc4oc5cc(-c6ccccc6)ccc5c4cc21)oc1cc(-c2ccccc2)ccc13 ; *n1c2cc3oc4cc(-c5ccccc5)ccc4c3cc2c2cc3c(cc21)oc1cc(-c2ccccc2)ccc13 ; *n1c2c(ccc3oc4cc(-c5ccccc5)ccc4c32)c2ccc3oc4cc(-c5ccccc5)ccc4c3c21 ; *n1c2c(ccc3c4ccc(-c5ccccc5)cc4oc32)c2ccc3c4ccc(-c5ccccc5)cc4oc3c21]

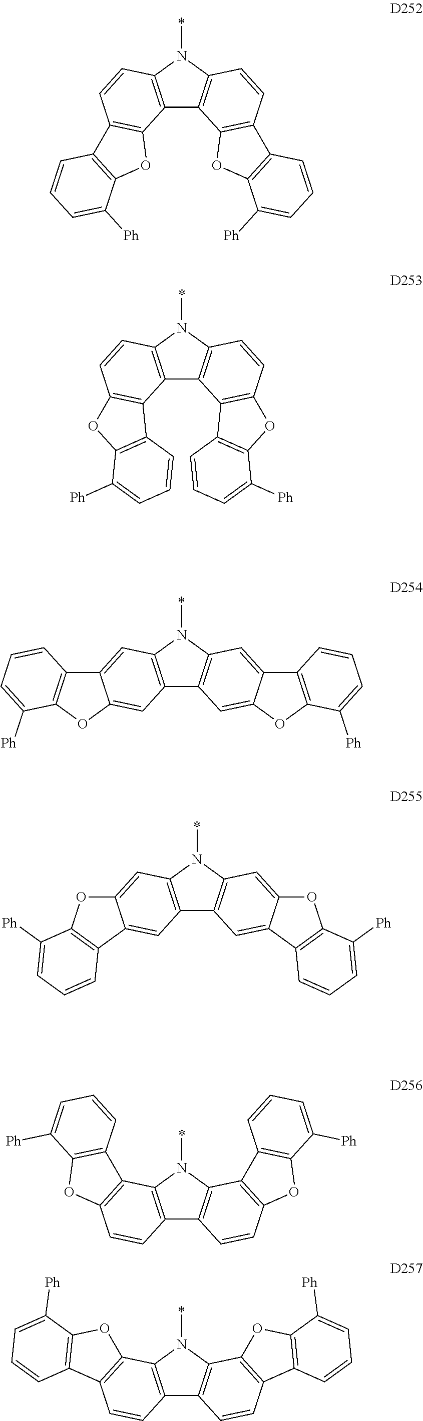 [SMILES: *n1c2ccc3c4cccc(-c5ccccc5)c4oc3c2c2c3oc4c(-c5ccccc5)cccc4c3ccc21 ; *n1c2ccc3oc4c(-c5ccccc5)cccc4c3c2c2c3c(ccc21)oc1c(-c2ccccc2)cccc13 ; *n1c2cc3c(cc2c2cc4oc5c(-c6ccccc6)cccc5c4cc21)oc1c(-c2ccccc2)cccc13 ; *n1c2cc3oc4c(-c5ccccc5)cccc4c3cc2c2cc3c(cc21)oc1c(-c2ccccc2)cccc13 ; *n1c2c(ccc3oc4c(-c5ccccc5)cccc4c32)c2ccc3oc4c(-c5ccccc5)cccc4c3c21 ; *n1c2c(ccc3c4cccc(-c5ccccc5)c4oc32)c2ccc3c4cccc(-c5ccccc5)c4oc3c21]

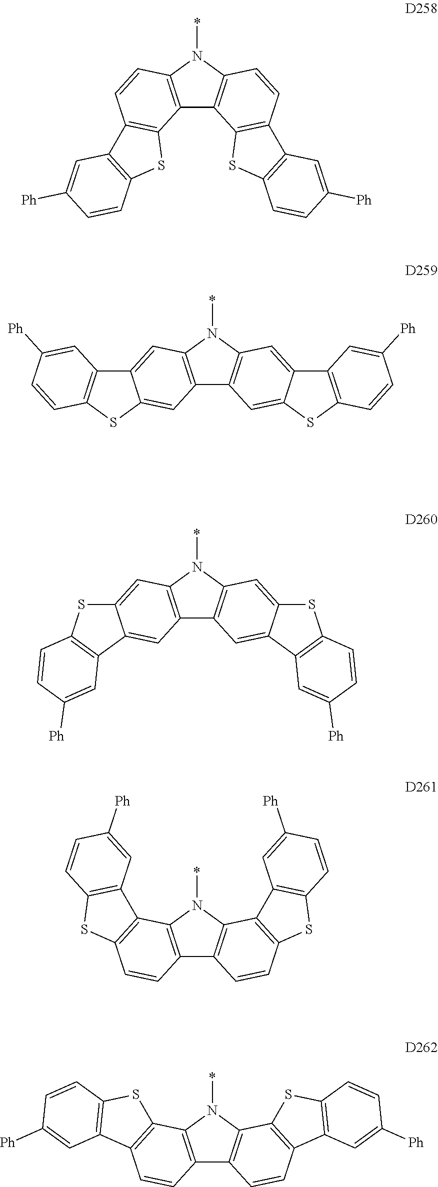 [SMILES: *n1c2ccc3c4cc(-c5ccccc5)ccc4sc3c2c2c3sc4ccc(-c5ccccc5)cc4c3ccc21 ; *n1c2cc3c(cc2c2cc4sc5ccc(-c6ccccc6)cc5c4cc21)sc1ccc(-c2ccccc2)cc13 ; *n1c2cc3sc4ccc(-c5ccccc5)cc4c3cc2c2cc3c(cc21)sc1ccc(-c2ccccc2)cc13 ; *n1c2c(ccc3sc4ccc(-c5ccccc5)cc4c32)c2ccc3sc4ccc(-c5ccccc5)cc4c3c21 ; *n1c2c(ccc3c4cc(-c5ccccc5)ccc4sc32)c2ccc3c4cc(-c5ccccc5)ccc4sc3c21]

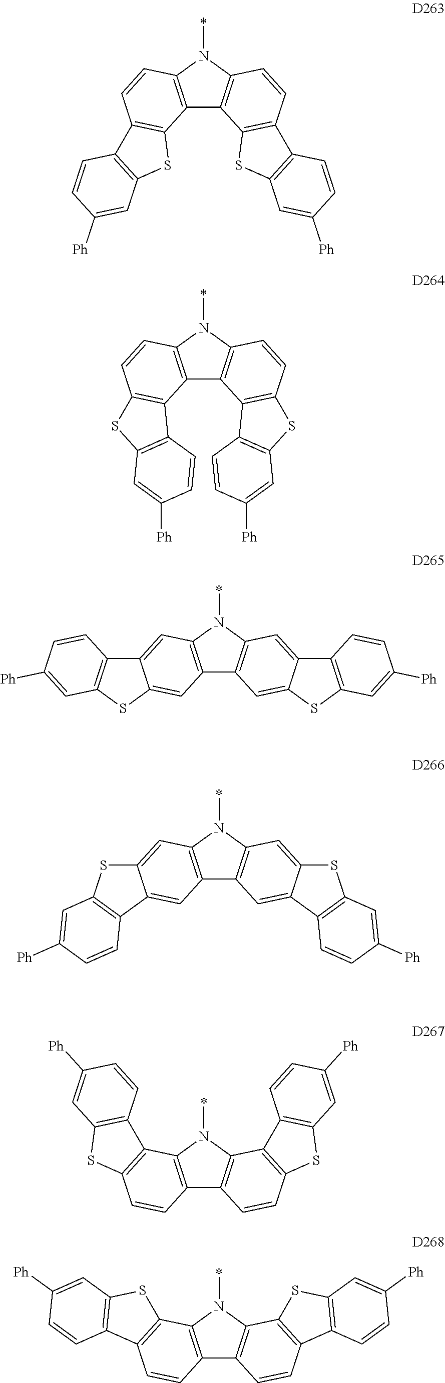 [SMILES: *n1c2ccc3c4ccc(-c5ccccc5)cc4sc3c2c2c3sc4cc(-c5ccccc5)ccc4c3ccc21 ; *n1c2ccc3sc4cc(-c5ccccc5)ccc4c3c2c2c3c(ccc21)sc1cc(-c2ccccc2)ccc13 ; *n1c2cc3c(cc2c2cc4sc5cc(-c6ccccc6)ccc5c4cc21)sc1cc(-c2ccccc2)ccc13 ; *n1c2cc3sc4cc(-c5ccccc5)ccc4c3cc2c2cc3c(cc21)sc1cc(-c2ccccc2)ccc13 ; *n1c2c(ccc3sc4cc(-c5ccccc5)ccc4c32)c2ccc3sc4cc(-c5ccccc5)ccc4c3c21 ; *n1c2c(ccc3c4ccc(-c5ccccc5)cc4sc32)c2ccc3c4ccc(-c5ccccc5)cc4sc3c21]

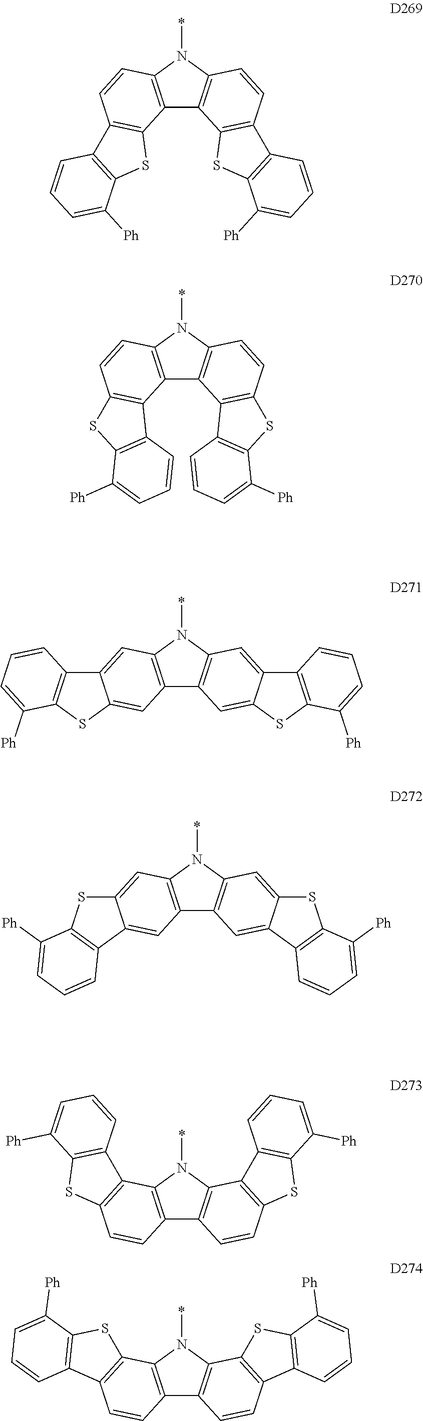 [SMILES: *n1c2ccc3c4cccc(-c5ccccc5)c4sc3c2c2c3sc4c(-c5ccccc5)cccc4c3ccc21 ; *n1c2ccc3sc4c(-c5ccccc5)cccc4c3c2c2c3c(ccc21)sc1c(-c2ccccc2)cccc13 ; *n1c2cc3c(cc2c2cc4sc5c(-c6ccccc6)cccc5c4cc21)sc1c(-c2ccccc2)cccc13 ; *n1c2cc3sc4c(-c5ccccc5)cccc4c3cc2c2cc3c(cc21)sc1c(-c2ccccc2)cccc13 ; *n1c2c(ccc3sc4c(-c5ccccc5)cccc4c32)c2ccc3sc4c(-c5ccccc5)cccc4c3c21 ; *n1c2c(ccc3c4cccc(-c5ccccc5)c4sc32)c2ccc3c4cccc(-c5ccccc5)c4sc3c21]

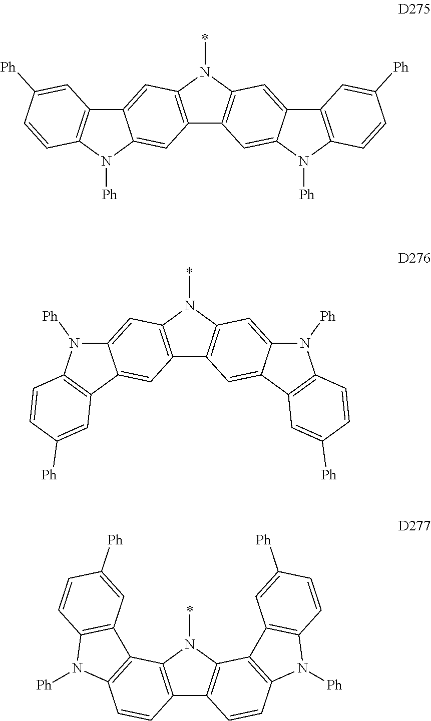 [SMILES: *n1c2cc3c4cc(-c5ccccc5)ccc4n(-c4ccccc4)c3cc2c2cc3c(cc21)c1cc(-c2ccccc2)ccc1n3-c1ccccc1 ; *n1c2cc3c(cc2c2cc4c5cc(-c6ccccc6)ccc5n(-c5ccccc5)c4cc21)c1cc(-c2ccccc2)ccc1n3-c1ccccc1 ; *n1c2c(ccc3c2c2cc(-c4ccccc4)ccc2n3-c2ccccc2)c2ccc3c(c4cc(-c5ccccc5)ccc4n3-c3ccccc3)c21]

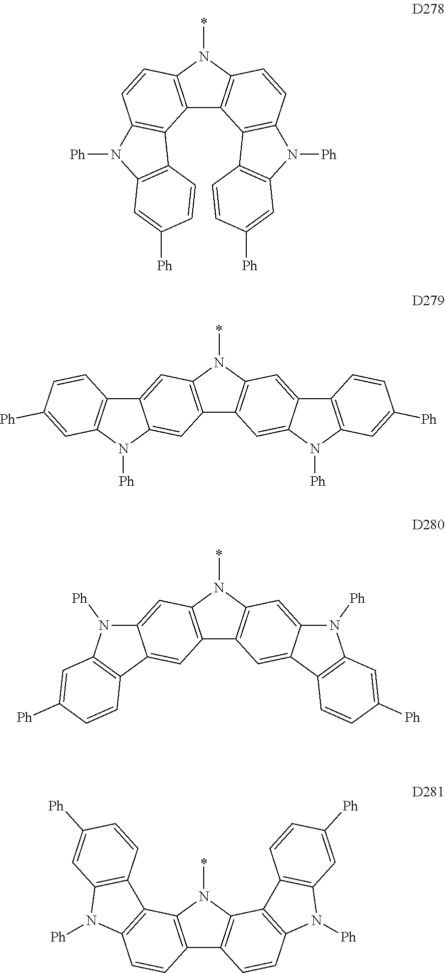 [SMILES: *n1c2ccc3c(c4ccc(-c5ccccc5)cc4n3-c3ccccc3)c2c2c3c4ccc(-c5ccccc5)cc4n(-c4ccccc4)c3ccc21 ; *n1c2cc3c4ccc(-c5ccccc5)cc4n(-c4ccccc4)c3cc2c2cc3c(cc21)c1ccc(-c2ccccc2)cc1n3-c1ccccc1 ; *n1c2cc3c(cc2c2cc4c5ccc(-c6ccccc6)cc5n(-c5ccccc5)c4cc21)c1ccc(-c2ccccc2)cc1n3-c1ccccc1 ; *n1c2c(ccc3c2c2ccc(-c4ccccc4)cc2n3-c2ccccc2)c2ccc3c(c4ccc(-c5ccccc5)cc4n3-c3ccccc3)c21]

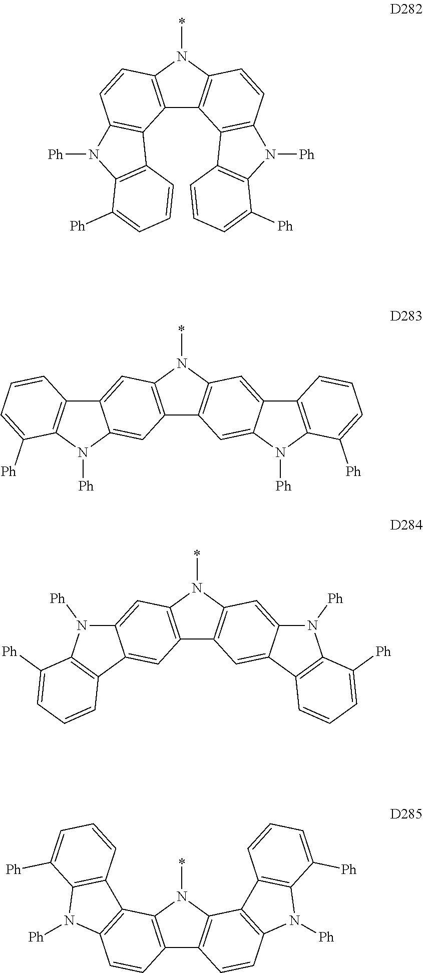 [SMILES: *n1c2ccc3c(c4cccc(-c5ccccc5)c4n3-c3ccccc3)c2c2c3c4cccc(-c5ccccc5)c4n(-c4ccccc4)c3ccc21 ; *n1c2cc3c4cccc(-c5ccccc5)c4n(-c4ccccc4)c3cc2c2cc3c(cc21)c1cccc(-c2ccccc2)c1n3-c1ccccc1 ; *n1c2cc3c(cc2c2cc4c5cccc(-c6ccccc6)c5n(-c5ccccc5)c4cc21)c1cccc(-c2ccccc2)c1n3-c1ccccc1 ; *n1c2c(ccc3c2c2cccc(-c4ccccc4)c2n3-c2ccccc2)c2ccc3c(c4cccc(-c5ccccc5)c4n3-c3ccccc3)c21]

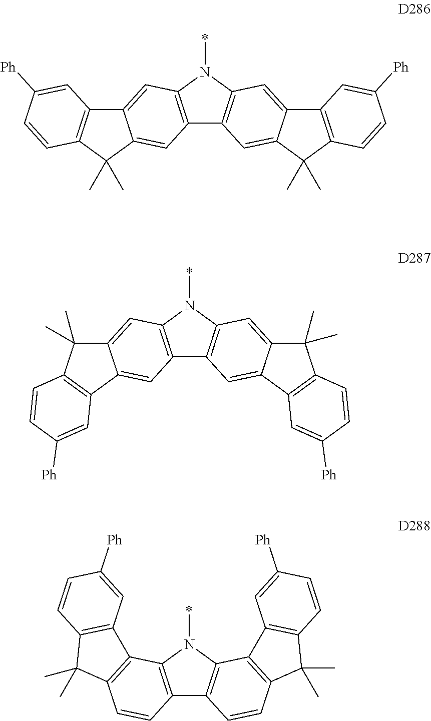

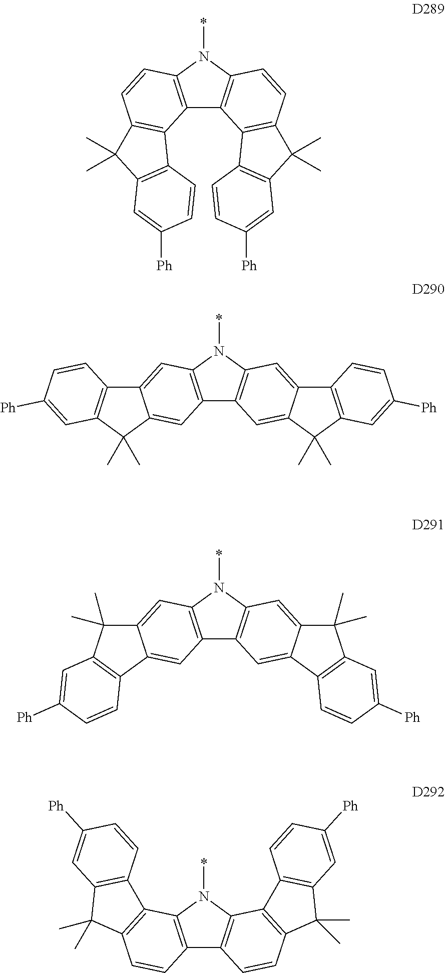

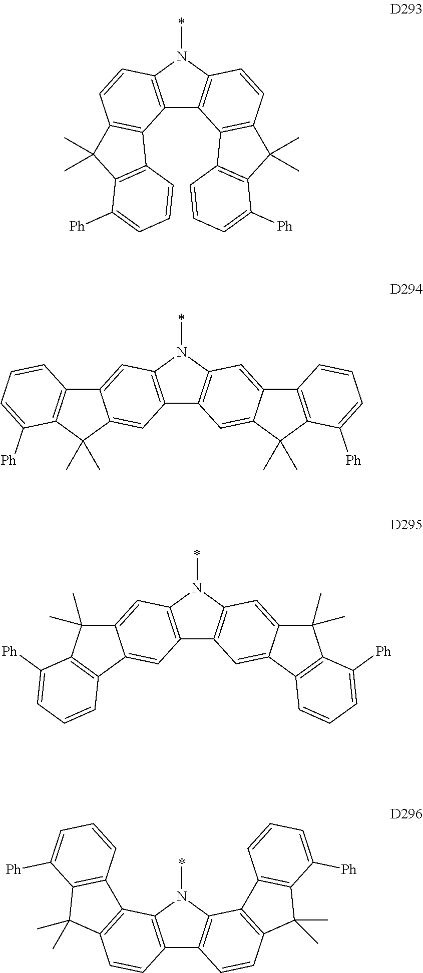

Groups obtained by substituting all hydrogen atoms present in the above D1 to D296 with deuterium atoms are disclosed as D297 to D592, respectively. Groups obtained by substituting all hydrogen atoms present in the phenyl group (Ph) of a substituent in the above D1 to D296 with deuterium atoms are disclosed as D593 to D888, respectively.

In one aspect of the present invention, the ring-fused carbazol-9-yl group in which the ring skeleton-constituting carbon atoms are substituted with at least two substituted or unsubstituted aryl groups is selected from D1 to D888. In one aspect of the present invention, the group is selected from D1 to D240, D297 to D536, and D593 to D832. In one aspect of the present invention, the group is selected from D241 to D296, D537 to D592, and D833 to D888. In one aspect of the present invention, the group is selected from D1 to 120, D241 to D274, D297 to D416, D537 to D570, D593 to D712, and D833 to D866. In one aspect of the present invention, the group is selected from D1 to 120, D297 to D416, and D593 to D712. In one aspect of the present invention, the group is selected from D1 to D60, D241 to D257, D297 to D356, D537 to D553, D593 to D652, and D833 to D849. In one aspect of the present invention, the group is selected from D61 to D120, D258 to D274, D357 to D416, D554 to D570, D653 to D712, and D850 to D866. In one aspect of the present invention, the group is selected from D121 to D180, D275 to D285, D417 to D476, D571 to D581, D713 to D772, and D867 to D877. In one aspect of the present invention, the group is selected from D181 to D240, D286 to D296, D477 to D536, D582 to D592, D773 to D832, and D878 to D888. In one aspect of the present invention, the group is selected from D1 to D18. In one aspect of the present invention, the group is selected from D19 to D36. In one aspect of the present invention, the group is selected from D37 to D42. In one aspect of the present invention, the group is selected from D43 to D60.

In the general formula (1), when only R¹ is the ring-fused carbazol-9-yl group in which the ring skeleton-constituting carbon atoms are substituted with at least two substituted or unsubstituted aryl groups, 0 or 1 of R² to R⁴ can be a ring-fused carbazol-9-yl group in which the ring skeleton-constituting carbon atoms are not substituted with at least two substituted or unsubstituted aryl groups. In that case, 0 or 1 ring-fused carbazol-9-yl group of R² to R⁴ can be further substituted with a substituent selected from Substituent Group A (excluding the case substituted with at least two substituted or unsubstituted aryl groups), or can be further substituted with a substituent selected from Substituent Group B (excluding the case substituted with at least two substituted or unsubstituted aryl groups), or can be further substituted with a substituent selected from Substituent Group C (excluding the case substituted with at least two substituted or unsubstituted aryl groups), or can be further substituted with a substituent selected from Substituent Group D (excluding the case substituted with at least two substituted or unsubstituted aryl groups), or can be further substituted with a substituent selected from Substituent Group E (excluding the case substituted with at least two substituted or unsubstituted aryl groups). In one aspect of the present invention, the group is a ring-fused carbazol-9-yl group optionally substituted with an alkyl group optionally substituted with a deuterium atom or an aryl group, or a deuterium atom, or with both the two. In one aspect of the present invention, the group is a ring-fused carbazol-9-yl group optionally further substituted with a deuterium atom. In one aspect of the present invention, the group is an unsubstituted ring-fused carbazol-9-yl group.

Specific examples of the ring-fused carbazol-9-yl group in which the ring skeleton-constituting carbon atoms are not substituted with at least two substituted or unsubstituted aryl groups and which can be employed as 0 or 1 of R² to R⁴ in the general formula (1) are shown below. However, the ring-fused carbazol-9-yl group in which the ring skeleton-constituting carbon atoms are not substituted with at least two substituted or unsubstituted aryl groups and which can be employed in the present invention shall not be construed as being limited by the following specific examples. In the following specific examples, * indicates a bonding site, and Ph represents a phenyl group. A methyl group is not shown. Accordingly, D895 to D906 have a methyl group.

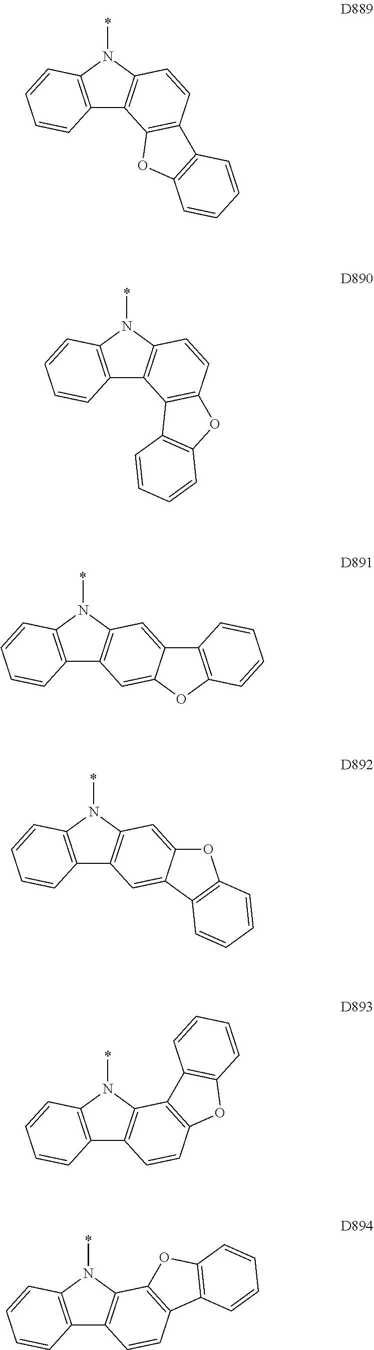 [SMILES: *n1c2ccccc2c2c3oc4ccccc4c3ccc21 ; *n1c2ccccc2c2c3c(ccc21)oc1ccccc13 ; *n1c2ccccc2c2cc3oc4ccccc4c3cc21 ; *n1c2ccccc2c2cc3c(cc21)oc1ccccc13 ; *n1c2ccccc2c2ccc3oc4ccccc4c3c21 ; *n1c2ccccc2c2ccc3c4ccccc4oc3c21]

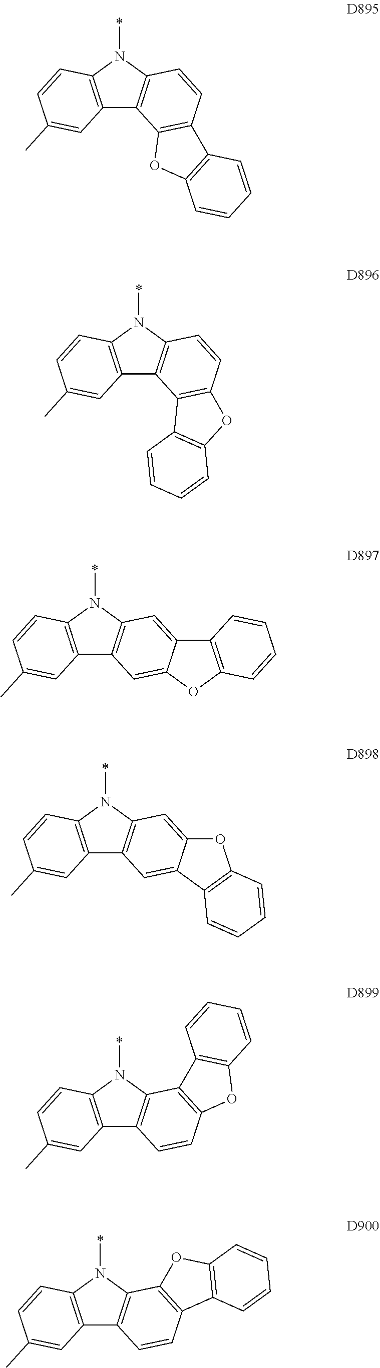 [SMILES: *n1c2ccc(C)cc2c2c3oc4ccccc4c3ccc21 ; *n1c2ccc(C)cc2c2c3c(ccc21)oc1ccccc13 ; *n1c2ccc(C)cc2c2cc3oc4ccccc4c3cc21 ; *n1c2ccc(C)cc2c2cc3c(cc21)oc1ccccc13 ; *n1c2ccc(C)cc2c2ccc3oc4ccccc4c3c21 ; *n1c2ccc(C)cc2c2ccc3c4ccccc4oc3c21]

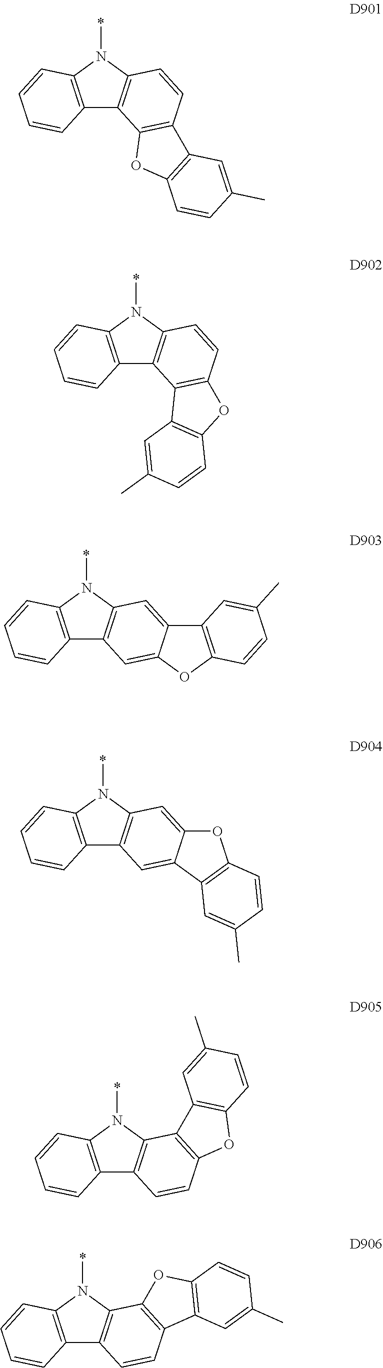 [SMILES: *n1c2ccccc2c2c3oc4ccc(C)cc4c3ccc21 ; *n1c2ccccc2c2c3c(ccc21)oc1ccc(C)cc13 ; *n1c2ccccc2c2cc3oc4ccc(C)cc4c3cc21 ; *n1c2ccccc2c2cc3c(cc21)oc1ccc(C)cc13 ; *n1c2ccccc2c2ccc3oc4ccc(C)cc4c3c21 ; *n1c2ccccc2c2ccc3c4cc(C)ccc4oc3c21]

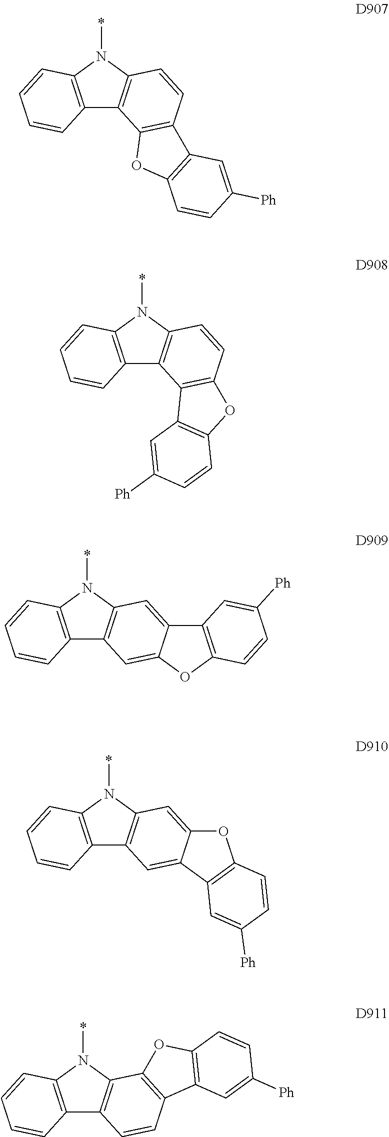 [SMILES: *n1c2ccccc2c2c3oc4ccc(-c5ccccc5)cc4c3ccc21 ; *n1c2ccccc2c2c3c(ccc21)oc1ccc(-c2ccccc2)cc13 ; *n1c2ccccc2c2cc3oc4ccc(-c5ccccc5)cc4c3cc21 ; *n1c2ccccc2c2cc3c(cc21)oc1ccc(-c2ccccc2)cc13 ; *n1c2ccccc2c2ccc3c4cc(-c5ccccc5)ccc4oc3c21]

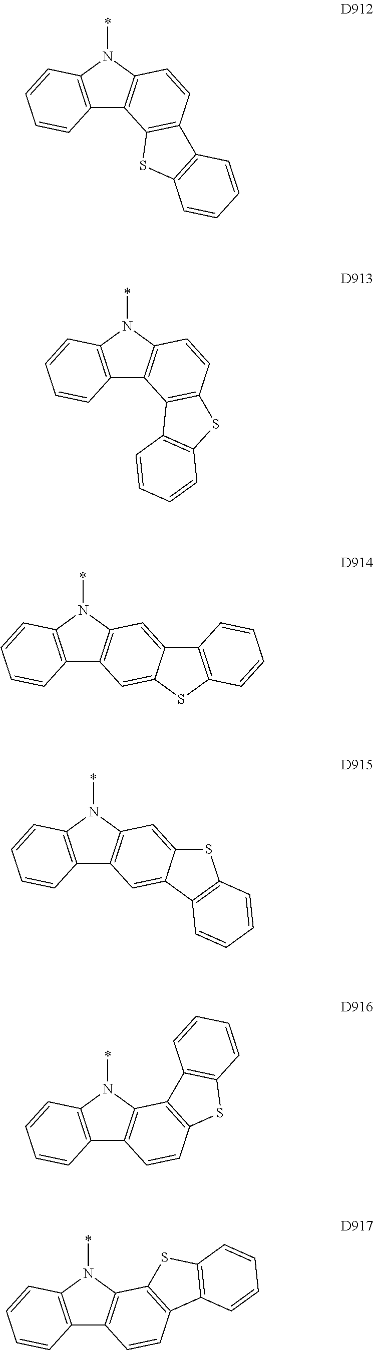 [SMILES: *n1c2ccccc2c2c3sc4ccccc4c3ccc21 ; *n1c2ccccc2c2c3c(ccc21)sc1ccccc13 ; *n1c2ccccc2c2cc3sc4ccccc4c3cc21 ; *n1c2ccccc2c2cc3c(cc21)sc1ccccc13 ; *n1c2ccccc2c2ccc3sc4ccccc4c3c21 ; *n1c2ccccc2c2ccc3c4ccccc4sc3c21]

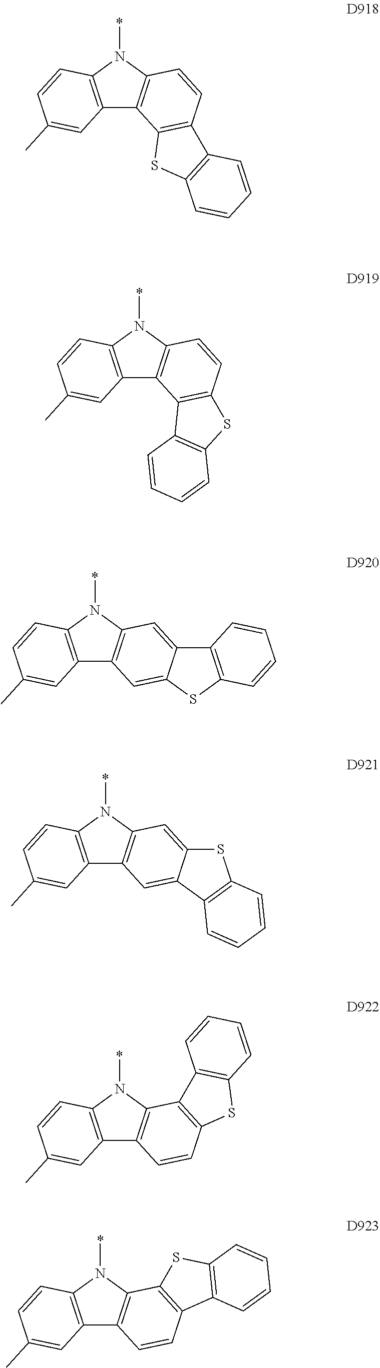 [SMILES: *n1c2ccc(C)cc2c2c3sc4ccccc4c3ccc21 ; *n1c2ccc(C)cc2c2c3c(ccc21)sc1ccccc13 ; *n1c2ccc(C)cc2c2cc3sc4ccccc4c3cc21 ; *n1c2ccc(C)cc2c2cc3c(cc21)sc1ccccc13 ; *n1c2ccc(C)cc2c2ccc3sc4ccccc4c3c21 ; *n1c2ccc(C)cc2c2ccc3c4ccccc4sc3c21]

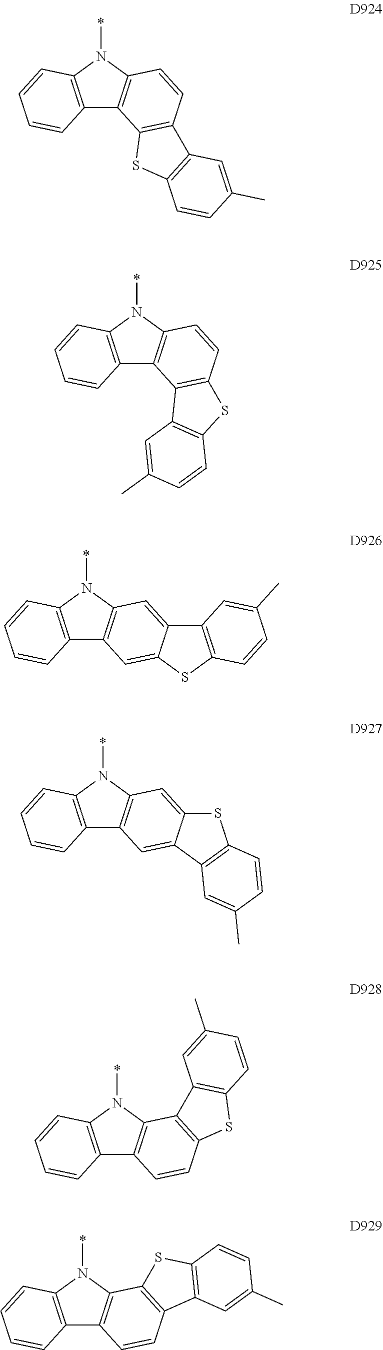 [SMILES: *n1c2ccccc2c2c3sc4ccc(C)cc4c3ccc21 ; *n1c2ccccc2c2c3c(ccc21)sc1ccc(C)cc13 ; *n1c2ccccc2c2cc3sc4ccc(C)cc4c3cc21 ; *n1c2ccccc2c2cc3c(cc21)sc1ccc(C)cc13 ; *n1c2ccccc2c2ccc3sc4ccc(C)cc4c3c21 ; *n1c2ccccc2c2ccc3c4cc(C)ccc4sc3c21]

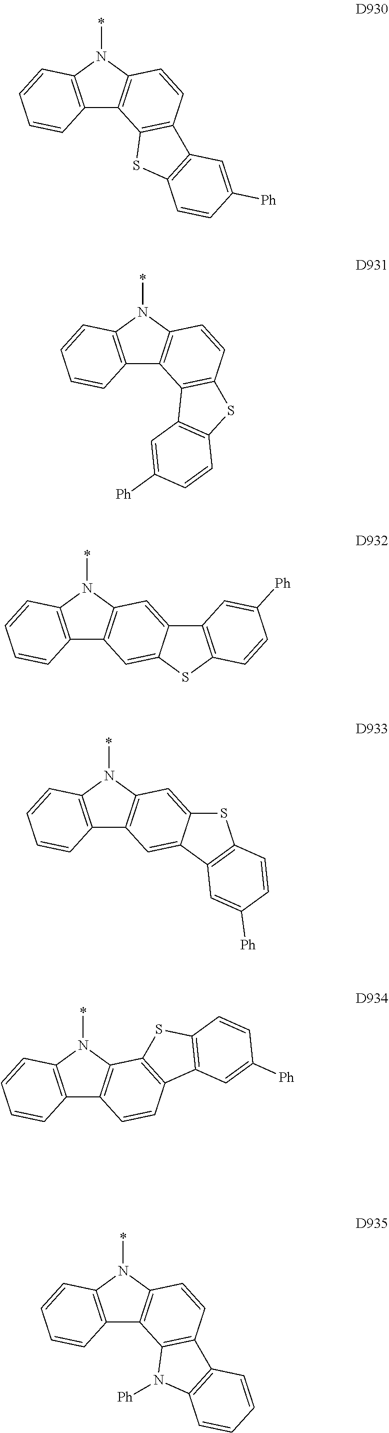 [SMILES: *n1c2ccccc2c2c3sc4ccc(-c5ccccc5)cc4c3ccc21 ; *n1c2ccccc2c2c3c(ccc21)sc1ccc(-c2ccccc2)cc13 ; *n1c2ccccc2c2cc3sc4ccc(-c5ccccc5)cc4c3cc21 ; *n1c2ccccc2c2cc3c(cc21)sc1ccc(-c2ccccc2)cc13 ; *n1c2ccccc2c2ccc3c4cc(-c5ccccc5)ccc4sc3c21 ; *n1c2ccccc2c2c1ccc1c3ccccc3n(-c3ccccc3)c12]

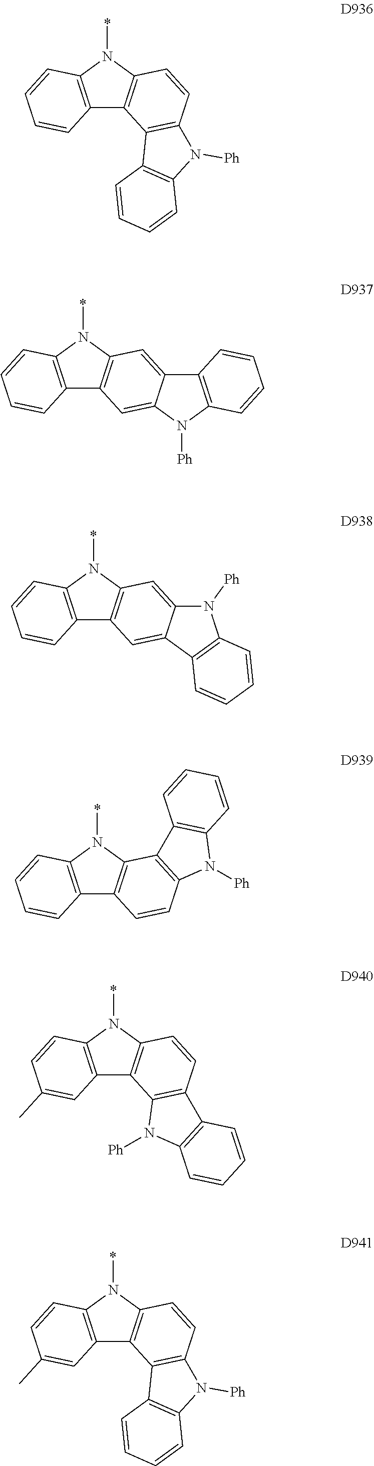 [SMILES: *n1c2ccccc2c2c3c4ccccc4n(-c4ccccc4)c3ccc21 ; *n1c2ccccc2c2cc3c(cc21)c1ccccc1n3-c1ccccc1 ; *n1c2ccccc2c2cc3c4ccccc4n(-c4ccccc4)c3cc21 ; *n1c2ccccc2c2ccc3c(c4ccccc4n3-c3ccccc3)c21 ; *n1c2ccc(C)cc2c2c1ccc1c3ccccc3n(-c3ccccc3)c12 ; *n1c2ccc(C)cc2c2c3c4ccccc4n(-c4ccccc4)c3ccc21]

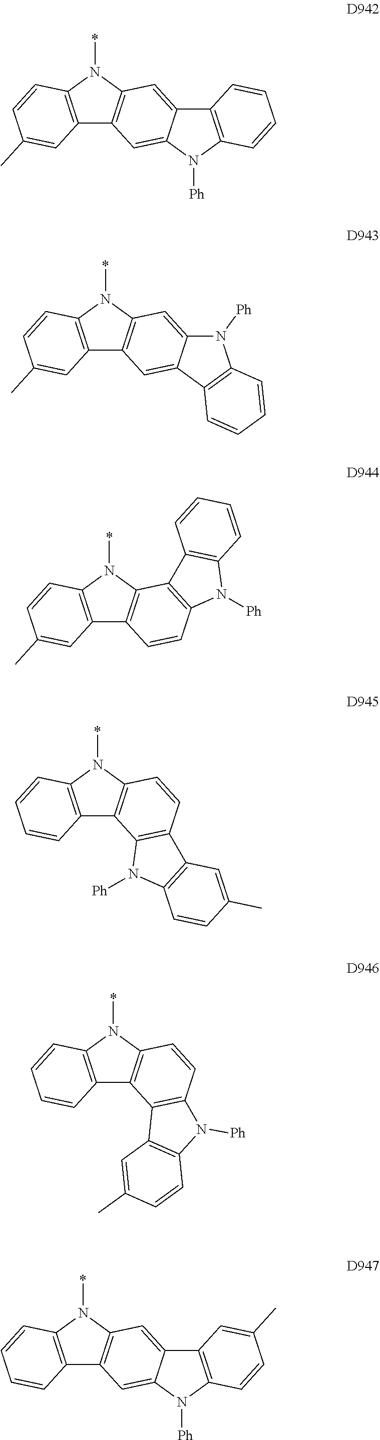 [SMILES: *n1c2ccc(C)cc2c2cc3c(cc21)c1ccccc1n3-c1ccccc1 ; *n1c2ccc(C)cc2c2cc3c4ccccc4n(-c4ccccc4)c3cc21 ; *n1c2ccc(C)cc2c2ccc3c(c4ccccc4n3-c3ccccc3)c21 ; *n1c2ccccc2c2c1ccc1c3cc(C)ccc3n(-c3ccccc3)c12 ; *n1c2ccccc2c2c3c4cc(C)ccc4n(-c4ccccc4)c3ccc21 ; *n1c2ccccc2c2cc3c(cc21)c1cc(C)ccc1n3-c1ccccc1]

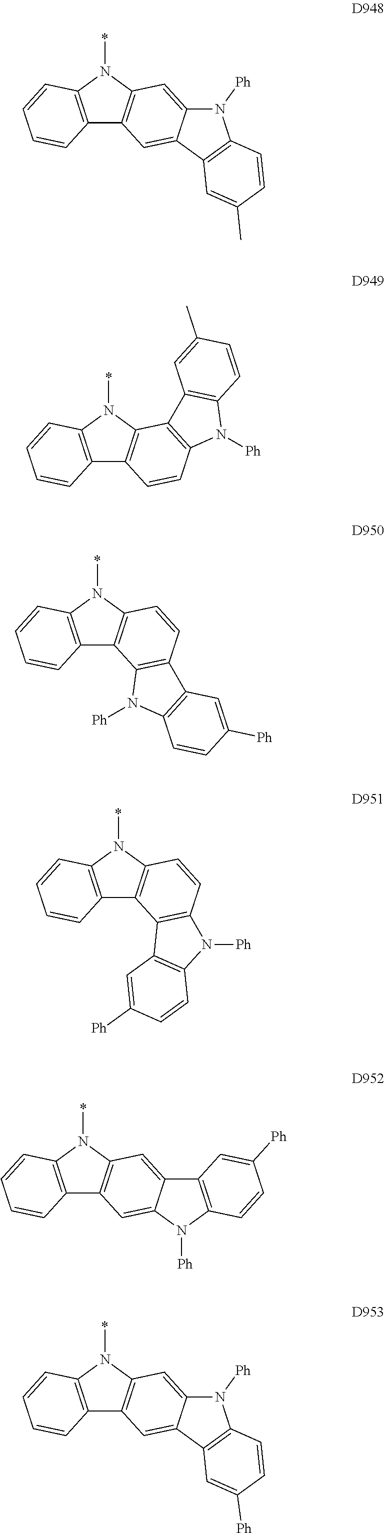 [SMILES: *n1c2ccccc2c2cc3c4cc(C)ccc4n(-c4ccccc4)c3cc21 ; *n1c2ccccc2c2ccc3c(c4cc(C)ccc4n3-c3ccccc3)c21 ; *n1c2ccccc2c2c1ccc1c3cc(-c4ccccc4)ccc3n(-c3ccccc3)c12 ; *n1c2ccccc2c2c3c4cc(-c5ccccc5)ccc4n(-c4ccccc4)c3ccc21 ; *n1c2ccccc2c2cc3c(cc21)c1cc(-c2ccccc2)ccc1n3-c1ccccc1 ; *n1c2ccccc2c2cc3c4cc(-c5ccccc5)ccc4n(-c4ccccc4)c3cc21]

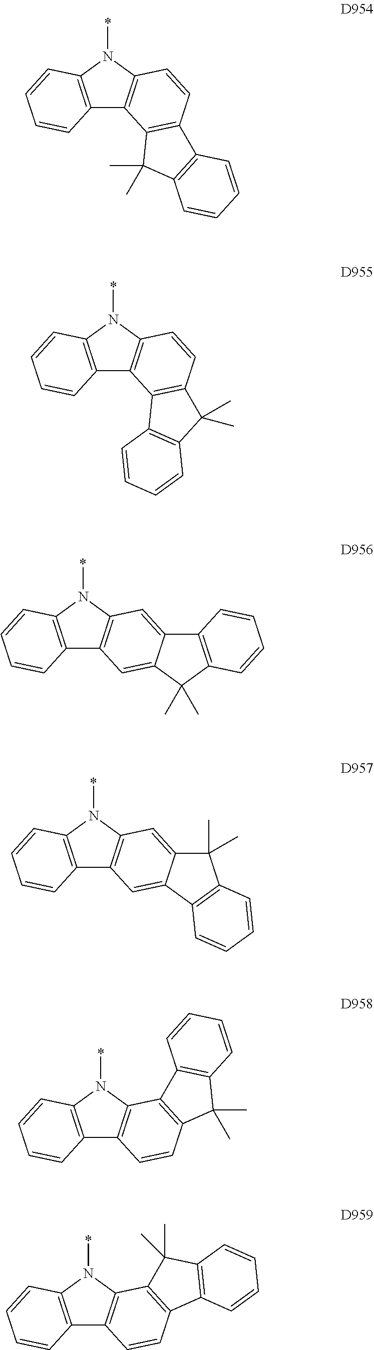 [SMILES: *n1c2ccccc2c2c3c(ccc21)-c1ccccc1C3(C)C ; *n1c2ccccc2c2c3c(ccc21)C(C)(C)c1ccccc1-3 ; *n1c2ccccc2c2cc3c(cc21)-c1ccccc1C3(C)C ; *n1c2ccccc2c2cc3c(cc21)C(C)(C)c1ccccc1-3 ; *n1c2ccccc2c2ccc3c(c21)-c1ccccc1C3(C)C ; *n1c2ccccc2c2ccc3c(c21)C(C)(C)c1ccccc1-3]

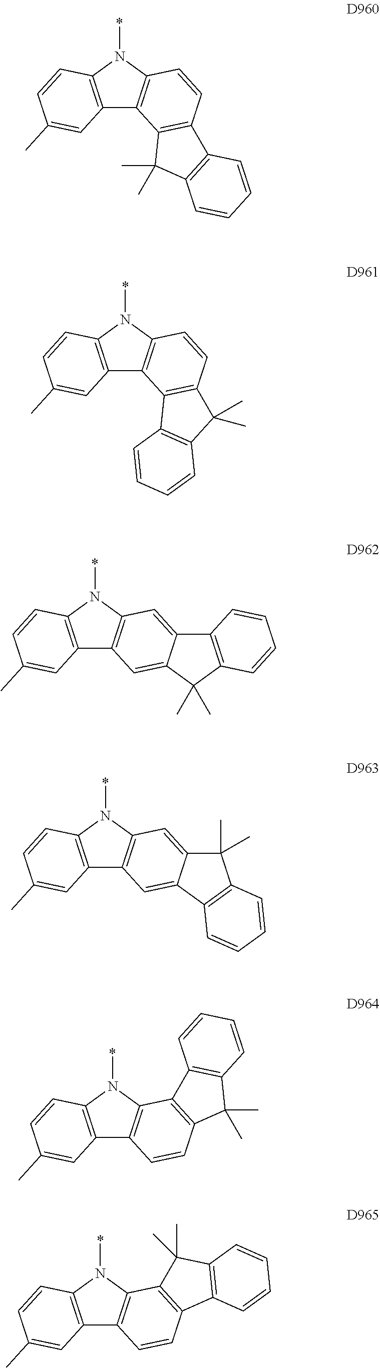

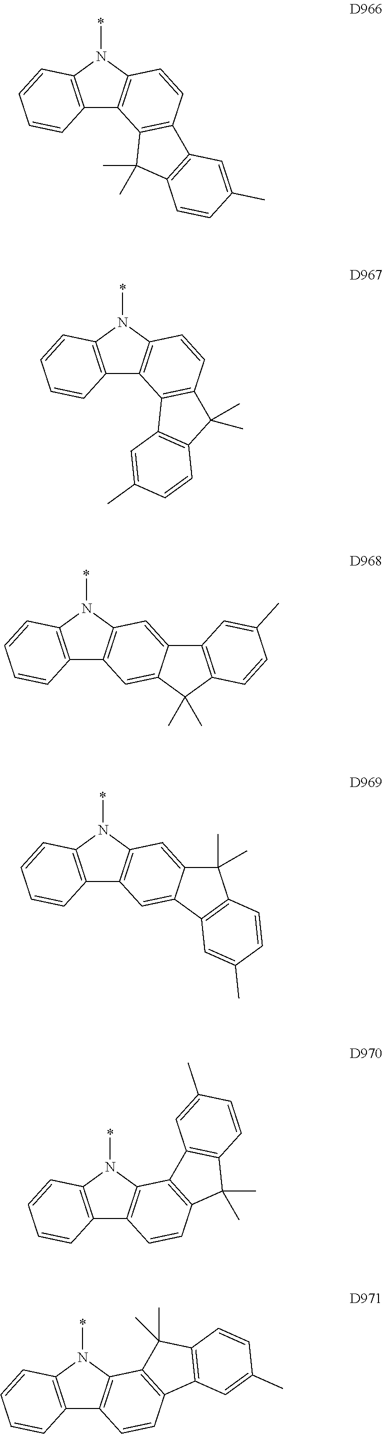 [SMILES: *n1c2ccccc2c2c3c(ccc21)-c1cc(C)ccc1C3(C)C ; *n1c2ccccc2c2c3c(ccc21)C(C)(C)c1ccc(C)cc1-3 ; *n1c2ccccc2c2cc3c(cc21)-c1cc(C)ccc1C3(C)C ; *n1c2ccccc2c2cc3c(cc21)C(C)(C)c1ccc(C)cc1-3 ; *n1c2ccccc2c2ccc3c(c21)-c1cc(C)ccc1C3(C)C ; *n1c2ccccc2c2ccc3c(c21)C(C)(C)c1ccc(C)cc1-3]

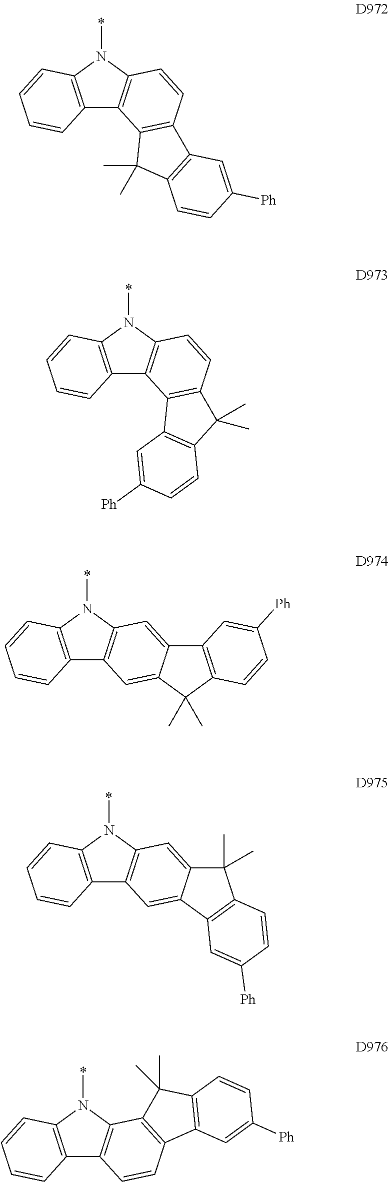 [SMILES: *n1c2ccccc2c2c3c(ccc21)-c1cc(-c2ccccc2)ccc1C3(C)C ; *n1c2ccccc2c2c3c(ccc21)C(C)(C)c1ccc(-c2ccccc2)cc1-3 ; *n1c2ccccc2c2cc3c(cc21)-c1cc(-c2ccccc2)ccc1C3(C)C ; *n1c2ccccc2c2cc3c(cc21)C(C)(C)c1ccc(-c2ccccc2)cc1-3 ; *n1c2ccccc2c2ccc3c(c21)C(C)(C)c1ccc(-c2ccccc2)cc1-3]

Groups obtained by substituting all hydrogen atoms present in the above D889 to D976 with deuterium atoms are disclosed as D977 to D1064, respectively. Groups obtained by substituting all hydrogen atoms present in the phenyl group and methyl group which are substituents for the above D895 to D911, and D918 to D976 with deuterium atoms are disclosed as D1065 to D1140, respectively.

In one aspect of the present invention, the ring-fused carbazol-9-yl group in which the ring skeleton-constituting carbon atoms are not substituted with at least two substituted or unsubstituted aryl groups is selected from D889 to D1140. In one aspect of the present invention, the group is selected from D889 to D953, D977 to D1041, and D1065 to D1117. In one aspect of the present invention, the group is selected from D889 to D934, D977 to D1022, and D1065 to D1098. In one aspect of the present invention, the group is selected from D889 to D911, D977 to D999, and D1065 to D1081. In one aspect of the present invention, the group is selected from D912 to D934, D1000 to D1022, and D1082 to D1098. In one aspect of the present invention, the group is selected from D935 to D953, D1023 to D1041, and D1099 to D1117. In one aspect of the present invention, the group is selected from D954 to D976, D1042 to D1064, and D1118 to D1140.

0 or 1 of R² to R⁴ in the general formula (1) is a substituted or unsubstituted ring-fused carbazol-9-yl group, and the other R² to R⁴ are each a substituted or unsubstituted non-ring-fused carbazol-9-yl group.

In one aspect of the present invention, any one of R² to R⁴ is a substituted or unsubstituted ring-fused carbazol-9-yl group, and the other R² to R⁴ (that is, 2 of R² to R⁴) are each independently a substituted or unsubstituted non-ring-fused carbazol-9-yl group. In one aspect of the present invention, R² is a substituted or unsubstituted ring-fused carbazol-9-yl group, and R³ and R⁴ are each independently a substituted or unsubstituted non-ring-fused carbazol-9-yl group. In one aspect of the present invention, R² is a ring-fused carbazol-9-yl group in which the ring skeleton-constituting carbon atoms are substituted with at least two substituted or unsubstituted aryl groups (where R¹ and R² can differ, but preferably R¹ and R² are the same), and R³ and R⁴ are each independently a substituted or unsubstituted non-ring-fused carbazol-9-yl group. In one aspect of the present invention, R² is a ring-fused carbazol-9-yl group in which the ring skeleton-constituting carbon atoms are not substituted with at least two substituted or unsubstituted aryl groups, and R³ and R⁴ are each independently a substituted or unsubstituted non-ring-fused carbazol-9-yl group. In one aspect of the present invention, R³ is a substituted or unsubstituted ring-fused carbazol-9-yl group, and R² and R⁴ are each independently a substituted or unsubstituted non-ring-fused carbazol-9-yl group. In one aspect of the present invention, R³ is a ring-fused carbazol-9-yl group in which the ring skeleton-constituting carbon atoms are substituted with at least two substituted or unsubstituted aryl groups (where R¹ and R³ can differ, but preferably R¹ and R³ are the same), and R² and R⁴ are each independently a substituted or unsubstituted non-ring-fused carbazol-9-yl group. In one aspect of the present invention, R³ is a ring-fused carbazol-9-yl group in which the ring skeleton-constituting carbon atoms are not substituted with at least two substituted or unsubstituted aryl groups, and R² and R⁴ are each independently a substituted or unsubstituted non-ring-fused carbazol-9-yl group. In one aspect of the present invention, R⁴ is a substituted or unsubstituted ring-fused carbazol-9-yl group, and R² and R³ are each independently a substituted or unsubstituted non-ring-fused carbazol-9-yl group. In one aspect of the present invention, R⁴ is a ring-fused carbazol-9-yl group in which the ring skeleton-constituting carbon atoms are substituted with at least two substituted or unsubstituted aryl groups (where R¹ and R⁴ can differ, but preferably R¹ and R⁴ are the same), and R² and R³ are each independently a substituted or unsubstituted non-ring-fused carbazol-9-yl group. In one aspect of the present invention, R⁴ is a ring-fused carbazol-9-yl group in which the ring skeleton-constituting carbon atoms are not substituted with at least two substituted or unsubstituted aryl groups, and R² and R³ are each independently a substituted or unsubstituted non-ring-fused carbazol-9-yl group.

In one aspect of the present invention, R², R³ and R⁴ are each independently a substituted or unsubstituted non-ring-fused carbazol-9-yl group.

2 or 3 substituted or unsubstituted non-ring-fused carbazol-9-yl groups which R² to R⁴ can represent can be substituted with a substituent selected from Substituent Group A, can be substituted with a substituent selected from Substituent Group B, can be substituted with a substituent selected from Substituent Group C, can be substituted with a substituent selected from Substituent Group D, and can be substituted with a substituent selected from Substituent Group E. In one aspect of the present invention, 2 or 3 non-ring-fused carbazol-9-yl groups which R² to R⁴ can represent are substituted with an aryl group optionally substituted with a deuterium atom or an alkyl group. In one aspect of the present invention, 2 or 3 non-ring-fused carbazol-9-yl groups which R² to R⁴ can represent are substituted with an alkyl group optionally substituted with a deuterium atom. In one aspect of the present invention, 2 or 3 non-ring-fused carbazol-9-yl groups which R² to R⁴ can represent are substituted with deuterium atoms or are unsubstituted. In one aspect of the present invention, 2 or 3 non-ring-fused carbazol-9-yl groups which R² to R⁴ can represent are all unsubstituted (that is, they are unsubstituted carbazol-9-yl groups).

In the case where the non-ring-fused carbazol-9-yl group is substituted, the substitution position of the substituent is not specifically limited. In one aspect of the present invention, each of the two benzene rings constituting the non-ring-fused carbazol-9-yl group is substituted with each one substituent. In one aspect of the present invention, only one of the two benzene rings constituting the non-ring-fused carbazol-9-yl group is substituted with a substituent. In one aspect of the present invention, the non-ring-fused carbazol-9-yl group has a substituent at the 3-position. In one aspect of the present invention, the non-ring-fused carbazol-9-yl group has a substituent at both the 3-position and the 6-position. In one aspect of the present invention, the non-ring-fused carbazol-9-yl group has a substituent only at the 3-position. In one aspect of the present invention, the non-ring-fused carbazol-9-yl group has a substituent only at both the 3-position and the 6-position.

2 or 3 substituted or unsubstituted non-ring-fused carbazol-9-yl groups which R² to R⁴ can represent can be the same as or different from each other.

In one preferred aspect of the present invention, R² and R³ are the same. In one preferred aspect of the present invention, R² and R⁴ are the same. In one preferred aspect of the present invention, R³ and R⁴ are the same. In one preferred aspect of the present invention, R², R³ and R⁴ are the same. In one preferred aspect of the present invention, R², R³ and R⁴ all differ.

Preferably, the substituted or unsubstituted non-ring-fused carbazol-9-yl group has a structure represented by the following general formula (3).

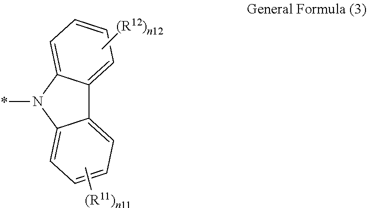

In the general formula (3), R¹¹ and R¹² each independently represent a substituent, in which the substituent includes a deuterium atom. Regarding the substituent, reference can be made to the description of the substituent of the substituted or unsubstituted non-ring-fused carbazol-9-yl group mentioned above.

In the general formula (3), n11 and n12 each independently represent an integer of 0 to 4. In one aspect of the present invention, when R¹¹ and R¹² are deuterium atoms, n11 and n12 are 4. When R¹¹ and R¹² are other substituents than deuterium atoms, n11 and n12 are preferably 0 to 2, for example, 0 or 1. In one aspect of the present invention, n11 and n12 are both 0.

In the general formula (3), R¹¹'s and R¹²'s each do not bond to each other to form a monocyclic structure. R¹¹ and R¹² also do not bond to each other to form a monocyclic structure.

One ring skeleton-constituting carbon atom at 1- to 4-positions of carbazole in the general formula (3) can be substituted with a nitrogen atom. One ring skeleton-constituting carbon atom at 5- to 8-positions of carbazole can also be substituted with a nitrogen atom. In one aspect of the present invention, one of the 1- to 4-positioned ring skeleton-constituting carbon atoms of carbazole is substituted with a nitrogen atom, and the 5- to 8-positioned ring skeleton-constituting carbon atoms of carbazole are not substituted with a nitrogen atom. For example, only the 1-positioned ring skeleton-constituting carbon atom is substituted with a nitrogen atom. For example, only the 2-positioned ring skeleton-constituting carbon atom is substituted with a nitrogen atom. For example, only the 3-positioned ring skeleton-constituting carbon atom is substituted with a nitrogen atom. For example, only the 4-positioned ring skeleton-constituting carbon atom is substituted with a nitrogen atom. For example, only the 3-positioned and 6-positioned ring skeleton-constituting carbon atoms are substituted with a nitrogen atom. In one aspect of the present invention, the 1- to 8-positioned ring skeleton-constituting carbon atoms of carbazole are not substituted with a nitrogen atom.

* in the general formula (3) indicates the bonding site to the benzene ring in the general formula (1).

In one preferred aspect of the present invention, R¹¹ and R¹² are each independently a substituent selected from Substituent Group E, n11+n12 is 0 to 4, preferably 0 to 2, including a case where n11 and n12 are both 1, a case where n11 is 1 and n12 is 0, and a case where n11 and n12 are both 0.

Specific examples of the substituted or unsubstituted non-ring-fused carbazol-9-yl group which R² to R⁴ can represent are shown below. However, the substituted or unsubstituted non-ring-fused carbazol-9-yl group which can be employed in the present invention shall not be construed as being limited by the following specific examples. In the following specific examples, * indicates a bonding site, and Ph represents a phenyl group. A methyl group is not shown. Consequently, Z2 and Z3 have an isopropyl group, and Z4 and Z5 have a methyl group.

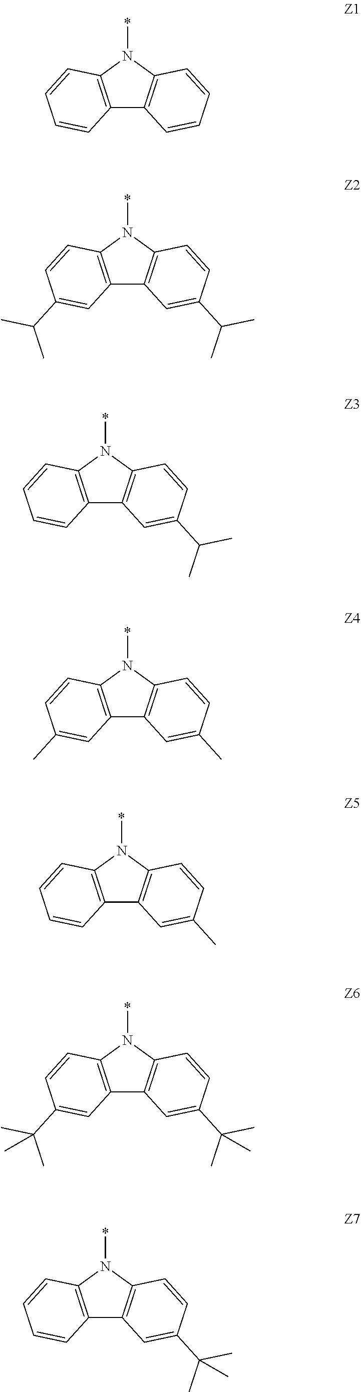

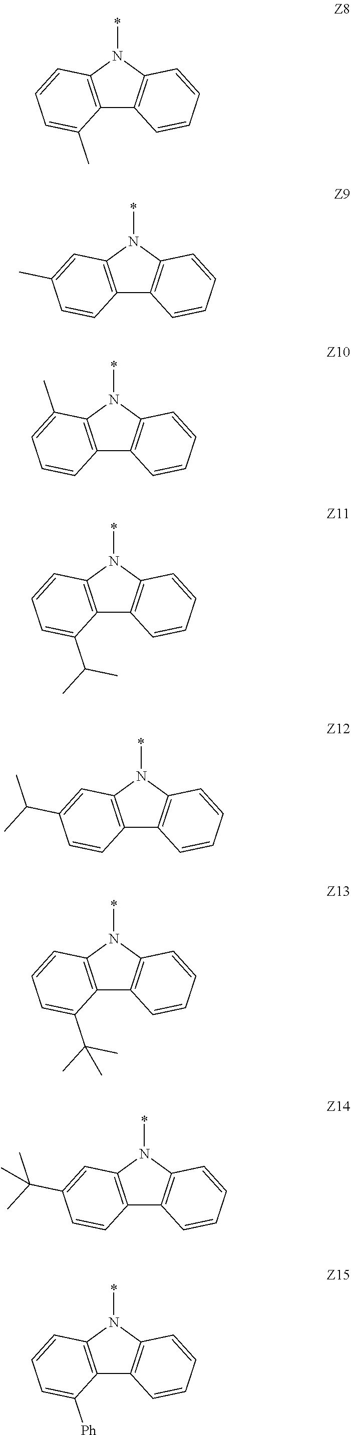

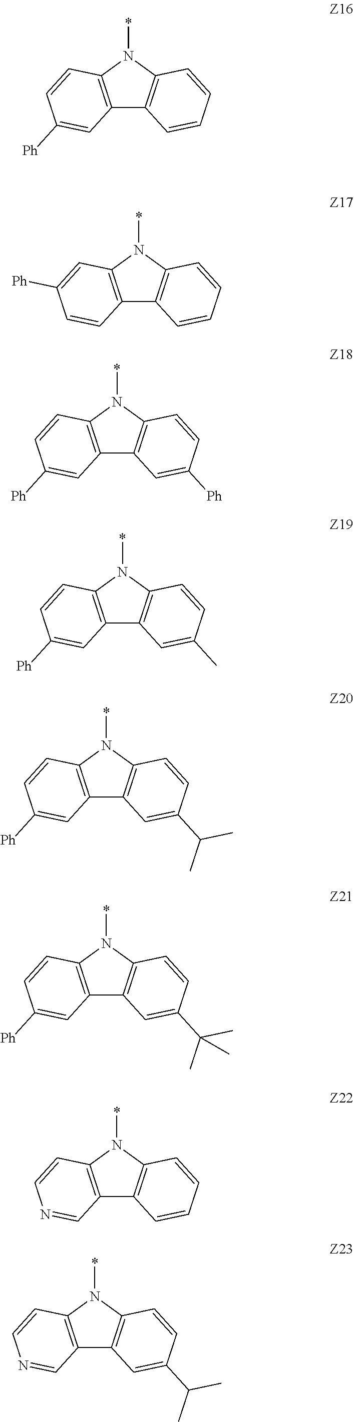

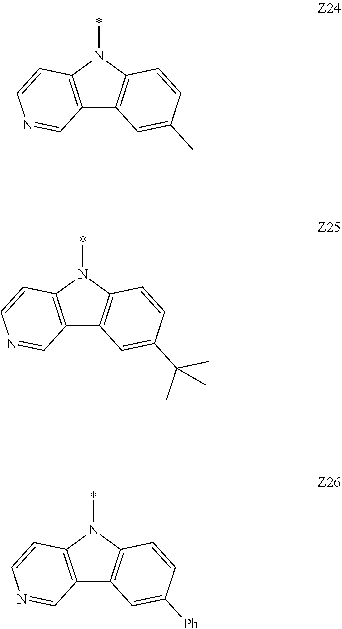 [SMILES: *n1c2ccncc2c2cc(C)ccc21 ; *n1c2ccncc2c2cc(C(C)(C)C)ccc21 ; *n1c2ccncc2c2cc(-c3ccccc3)ccc21]

Groups obtained by substituting all hydrogen atoms present in the above Z1 to Z26 with deuterium atoms are disclosed as Z27 to Z52, respectively. Groups obtained by substituting all hydrogen atoms present in the phenyl group and the alkyl group which are substituents for the above Z2 to Z21 and Z23 to Z26 with deuterium atoms are disclosed as Z53 to Z76, respectively.

In one aspect of the present invention, the substituted or unsubstituted non-ring-fused carbazol-9-yl group is selected from Z1 to Z76. In one aspect of the present invention, the group is selected from Z1 to Z21, Z27 to Z47, and Z53 to Z72. In one aspect of the present invention, the group is selected from Z1 to Z21, Z27 to Z47, and Z53 to Z72. In one aspect of the present invention, the group is selected from Z1 to Z14, Z27 to Z40, and Z53 to Z65. In one aspect of the present invention, the group is selected from Z15 to Z21, Z41 to Z47, and Z66 to Z72.

In one preferred aspect of the present invention, R² to R⁴ are the same, and each is preferably a non-ring-fused carbazol-9-yl group optionally substituted with a substituent selected from Substituent Group E, for example, a non-ring-fused carbazol-9-yl group optionally substituted with a deuterium atom.

In one aspect of the present invention, R² to R⁴ each are a substituted or unsubstituted non-ring-fused carbazol-9-yl group, and R² and R³ are the same. In one aspect of the present invention, R² to R⁴ each are a substituted or unsubstituted non-ring-fused carbazol-9-yl group, and R² and R⁴ are the same. In one aspect of the present invention, R² to R⁴ each are a substituted or unsubstituted non-ring-fused carbazol-9-yl group, and R³ and R⁴ are the same. In one aspect of the present invention, R² to R⁴ each are a substituted or unsubstituted non-ring-fused carbazol-9-yl group, and R² to R⁴ are different from each other.

In one aspect of the present invention, R¹ and R² are the same, and R³ and R⁴ are also the same. In one aspect of the present invention, R¹ and R² are the same, and R³ and R⁴ differ. In one aspect of the present invention, R¹ and R³ are the same, and R² and R⁴ are also the same. In one aspect of the present invention, R¹ and R³ are the same, and R² and R⁴ differ. In one aspect of the present invention, R¹ and R⁴ are the same, and R² and R³ are also the same. In one aspect of the present invention, R¹ and R⁴ are the same, and R² and R³ differ.

In one aspect of the present invention, R² is a ring-fused carbazol-9-yl group in which the ring skeleton-constituting carbon atoms are not substituted with at least two substituted or unsubstituted aryl groups, and R³ and R⁴ are the same. In one aspect of the present invention, R² is a ring-fused carbazol-9-yl group in which the ring skeleton-constituting carbon atoms are not substituted with at least two substituted or unsubstituted aryl groups, and R³ and R⁴ differ. In one aspect of the present invention, R³ is a ring-fused carbazol-9-yl group in which the ring skeleton-constituting carbon atoms are not substituted with at least two substituted or unsubstituted aryl groups, and R² and R⁴ are the same. In one aspect of the present invention, R³ is a ring-fused carbazol-9-yl group in which the ring skeleton-constituting carbon atoms are not substituted with at least two substituted or unsubstituted aryl groups, and R² and R⁴ differ. In one aspect of the present invention, R⁴ is a ring-fused carbazol-9-yl group in which the ring skeleton-constituting carbon atoms are not substituted with at least two substituted or unsubstituted aryl groups, and R² and R³ are the same. In one aspect of the present invention, R⁴ is a ring-fused carbazol-9-yl group in which the ring skeleton-constituting carbon atoms are not substituted with at least two substituted or unsubstituted aryl groups, and R² and R³ differ.

In one preferred aspect of the present invention, the “substituted or unsubstituted aryl group” of the “ring-fused carbazol-9-yl group in which the ring skeleton-constituting carbon atoms are substituted with at least two substituted or unsubstituted aryl groups” in R¹ is an aryl group in which at least one hydrogen atom existing in the group is substituted with a deuterium atom, more preferably an aryl group in which all the hydrogen atoms are substituted with deuterium atoms, and further preferably a phenyl group in which all the hydrogen atoms existing in the group are substituted with deuterium atoms.

In one preferred aspect of the present invention, the “ring-fused carbazol-9-yl group” of the “ring-fused carbazol-9-yl group in which the ring skeleton-constituting carbon atoms are substituted with at least two substituted or unsubstituted aryl groups” in R¹ is a benzofuran ring-fused carbazol-9-yl group, and the “substituted or unsubstituted aryl group” with which the ring-fused carbazol-9-yl group is substituted is an aryl group in which at least one hydrogen atom existing in the group is substituted with a deuterium atom. In one more preferred aspect of the present invention, R¹ is a group represented by the following general formula (2a).

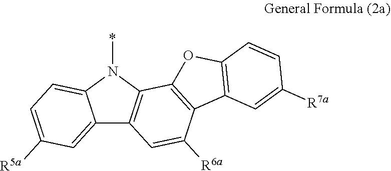

In the general formula (2a), R^{5a} to R^{7a} each independently represent a hydrogen atom, or an aryl group in which at least one hydrogen atom existing in the group is substituted with a deuterium atom, and at least two of R^{5a} to R^{7a} each are an aryl group in which at least one hydrogen atom is substituted with a deuterium atom. * indicates a bonding site. Here, preferably, R^{5a} and R^{6a} each are an aryl group in which at least one hydrogen atom is substituted with a deuterium atom and R^{7a} is a hydrogen atom, and also preferably, R^{5a} is a hydrogen atom and R^{6a} and R^{7a} each are an aryl group in which at least one hydrogen atom is substituted with a deuterium atom. The aryl group in which at least one hydrogen atom is substituted with a deuterium atom is preferably an aryl group in which all hydrogen atoms existing therein are substituted with deuterium atoms, and more preferably a phenyl group in which all hydrogen atoms existing therein are substituted with deuterium atoms.

In one preferred aspect of the present invention, the “ring-fused carbazol-9-yl group” in the “ring-fused carbazol-9-yl group in which the ring skeleton-constituting carbon atoms are substituted with at least two substituted or unsubstituted aryl groups” in R¹ is a benzothiophene ring-fused carbazol-9-yl group, and the “substituted or unsubstituted aryl group” with which the ring-fused carbazol-9-yl group is substituted is an aryl group in which at least one hydrogen atom existing therein is substituted with a deuterium atom. In one more preferred aspect of the present invention, R¹ is a group represented by the following general formula (2b),

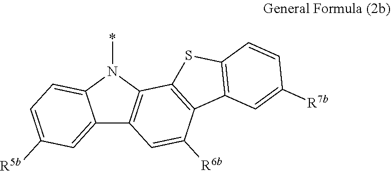

In the general formula (2b), R^{5b} to R^{7b} each independently represent a hydrogen atom, or an aryl group in which at least one hydrogen atom existing in the group is substituted with a deuterium atom, and at least two of R^{5b} to R^{7b} each are an aryl group in which at least one hydrogen atom is substituted with a deuterium atom. * indicates a bonding site. Here, preferably, R^{5b} and R^{6b} each are an aryl group in which at least one hydrogen atom is substituted with a deuterium atom and R^{7b} is a hydrogen atom, and also preferably, R^{5b} is a hydrogen atom and R^{6b} and R^{7b} each are an aryl group in which at least one hydrogen atom is substituted with a deuterium atom. The aryl group in which at least one hydrogen atom is substituted with a deuterium atom is preferably an aryl group in which all hydrogen atoms existing therein are substituted with deuterium atoms, more preferably a phenyl group in which all hydrogen atoms existing therein are substituted with deuterium atoms.

In one preferred aspect of the present invention, R² to R⁴ each are a non-ring-fused carbazol-9-yl group in which at least one hydrogen atom existing therein is substituted with a deuterium atom, and more preferably a non-ring-fused carbazol-9-yl group in which all hydrogen atoms existing therein are substituted with deuterium atoms. In one preferred aspect of the present invention, R² to R⁴ each are a non-ring-fused carbazol-9-yl group in which at least one hydrogen atom is substituted with a deuterium atom, and have the same structure.

In one preferred aspect of the present invention, the “substituted or unsubstituted aryl group” of the “ring-fused carbazol-9-yl group in which the ring skeleton-constituting carbon atoms are substituted with at least two substituted or unsubstituted aryl groups” in R¹ is an aryl group in which at least one hydrogen atom existing in the group is substituted with a deuterium atom, and R² to R⁴ each are a non-ring-fused carbazol-9-yl group in which at least one hydrogen atom existing in the group is substituted with a deuterium atom.

The compound represented by the general formula (1) preferably does not contain a metal atom, and can be a compound composed only of atoms selected from the group consisting of a carbon atom, a hydrogen atom, a deuterium atom, a nitrogen atom, an oxygen atom, and a sulfur atom. In one preferred aspect of the present invention, the compound represented by the general formula (1) is composed only of atoms selected from the group consisting of a carbon atom, a hydrogen atom, a deuterium atom, a nitrogen atom, and an oxygen atom. In addition, the compound represented by the general formula (1) can be a compound composed only of atoms selected from the group consisting of a carbon atom, a hydrogen atom, a deuterium atom, a nitrogen atom, and a sulfur atom. The compound represented by the general formula (1) can be a compound composed only of atoms selected from the group consisting of a carbon atom, a hydrogen atom, a deuterium atom, and a nitrogen atom. Further, the compound represented by the general formula (1) can be a compound which contains a deuterium atom.

In the description herein, the term “Substituent Group A” means one atom or group or a combination of two or more thereof selected from the group consisting of a deuterium atom, a hydroxyl group, a halogen atom (for example, a fluorine atom, a chlorine atom, a bromine atom, and an iodine atom), an alkyl group (for example, having 1 to 40 carbon atoms), an alkoxy group (for example, having 1 to 40 carbon atoms), an alkylthio group (for example, having 1 to 40 carbon atoms), an aryl group (for example, having 6 to 30 carbon atoms), an aryloxy group (for example, having 6 to 30 carbon atoms), an arylthio group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), a heteroarylthio group (for example, having 5 to 30 ring skeleton-constituting atoms), an acyl group (for example, having 1 to 40 carbon atoms), an alkenyl group (for example, having 1 to 40 carbon atoms), an alkynyl group (for example, having 1 to 40 carbon atoms), an alkoxycarbonyl group (for example, having 1 to 40 carbon atoms), an aryloxycarbonyl group (for example, having 1 to 40 carbon atoms), a heteroaryloxycarbonyl group (for example, having 1 to 40 carbon atoms), a silyl group (for example, a trialkylsilyl group having 1 to 40 carbon atoms), and a nitro group.

In the description herein, the term “Substituent Group B” means one atom or group or a combination of two or more thereof selected from the group consisting of a deuterium atom, an alkyl group (for example, having 1 to 40 carbon atoms), an alkoxy group (for example, having 1 to 40 carbon atoms), an aryl group (for example, having 6 to 30 carbon atoms), an aryloxy group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), and a diarylaminoamino group (for example, having 0 to 20 carbon atoms).

In the description herein, the term “Substituent Group C” means one atom or group or a combination of two or more thereof selected from the group consisting of a deuterium atom, an alkyl group (for example, having 1 to 20 carbon atoms), an aryl group (for example, having 6 to 22 carbon atoms), a heteroaryl group (for example, having 5 to 20 ring skeleton-constituting atoms), and a diarylamino group (for example, having 12 to 20 carbon atoms).

In the description herein, the term “Substituent Group D” means one atom or group or a combination of two or more thereof selected from the group consisting of a deuterium atom, an alkyl group (for example, having 1 to 20 carbon atoms), an aryl group (for example, having 6 to 22 carbon atoms), and a heteroaryl group (for example, having 5 to 20 ring skeleton-constituting atoms).

In the description herein, the term “Substituent Group E” means one atom or group or a combination of two or more groups selected from the group consisting of a deuterium atom, an alkyl group (for example, having 1 to 20 carbon atoms), and an aryl group (for example, having 6 to 22 carbon atoms).

In the description herein, the substituent meant by an expression of “substituted or unsubstituted” or “optionally substituted” can be selected, for example, from Substituent Group A, can be selected from Substituent Group B, can be selected from Substituent Group C, can be selected from Substituent Group D, or can be selected from Substituent Group E.

Specific examples of the compound represented by the general formula (1) are shown in the following Tables 1 to 3. However, the compound represented by the general formula (1) that can be used in the present invention should not be construed as being limited by these specific examples.

In Table 1, structures of Compounds 1 to 888 are individually shown by specifying R¹ to R⁴ of the general formula (1) for each compound. For example, Compound 1 is a compound where R¹ is D1 and R² to R⁴ are Z1.

In Table 2, structures of Compounds 1 to 67488 are shown by collectively displaying R¹ to R⁴ of a plurality of compounds in each row. For example, in the row of Compounds 1 to 888 in Table 2, compounds in which R² to R⁴ are fixed to Z1, and R¹ is D1 to D888 are referred to as Compounds 1 to 888 in that order. That is, the row of Compounds 1 to 888 in Table 2 collectively represents Compounds 1 to 888 specified in Table 1. Similarly, in the row of Compounds 889 to 1776 in Table 2, those in which R² to R⁴ are fixed to Z2 and R¹ is D1 to D888 are referred to as Compounds 889 to 1776 in that order. In the same manner, Compounds 1777 to 67488 in Table 2 are also specified.

TABLE 1

| No. | R¹ | R² | R³ | R⁴ |
|---|---|---|---|---|
| 1 | D1 | Z1 | Z1 | Z1 |
| 2 | D2 | Z1 | Z1 | Z1 |
| 3 | D3 | Z1 | Z1 | Z1 |
| 4 | D4 | Z1 | Z1 | Z1 |
| 5 | D5 | Z1 | Z1 | Z1 |
| 6 | D6 | Z1 | Z1 | Z1 |
| 7 | D7 | Z1 | Z1 | Z1 |
| 8 | D8 | Z1 | Z1 | Z1 |
| 9 | D9 | Z1 | Z1 | Z1 |
| 10 | D10 | Z1 | Z1 | Z1 |
| 11 | D11 | Z1 | Z1 | Z1 |
| 12 | D12 | Z1 | Z1 | Z1 |
| 13 | D13 | Z1 | Z1 | Z1 |
| 14 | D14 | Z1 | Z1 | Z1 |
| 15 | D15 | Z1 | Z1 | Z1 |
| 16 | D16 | Z1 | Z1 | Z1 |
| 17 | D17 | Z1 | Z1 | Z1 |
| 18 | D18 | Z1 | Z1 | Z1 |
| 19 | D19 | Z1 | Z1 | Z1 |
| 20 | D20 | Z1 | Z1 | Z1 |
| 21 | D21 | Z1 | Z1 | Z1 |
| 22 | D22 | Z1 | Z1 | Z1 |
| 23 | D23 | Z1 | Z1 | Z1 |
| 24 | D24 | Z1 | Z1 | Z1 |
| 25 | D25 | Z1 | Z1 | Z1 |
| 26 | D26 | Z1 | Z1 | Z1 |
| 27 | D27 | Z1 | Z1 | Z1 |
| 28 | D28 | Z1 | Z1 | Z1 |
| 29 | D29 | Z1 | Z1 | Z1 |
| 30 | D30 | Z1 | Z1 | Z1 |
| 31 | D31 | Z1 | Z1 | Z1 |
| 32 | D32 | Z1 | Z1 | Z1 |
| 33 | D33 | Z1 | Z1 | Z1 |
| 34 | D34 | Z1 | Z1 | Z1 |
| 35 | D35 | Z1 | Z1 | Z1 |
| 36 | D36 | Z1 | Z1 | Z1 |
| 37 | D37 | Z1 | Z1 | Z1 |
| 38 | D38 | Z1 | Z1 | Z1 |
| 39 | D39 | Z1 | Z1 | Z1 |
| 40 | D40 | Z1 | Z1 | Z1 |
| 41 | D41 | Z1 | Z1 | Z1 |
| 42 | D42 | Z1 | Z1 | Z1 |
| 43 | D43 | Z1 | Z1 | Z1 |
| 44 | D44 | Z1 | Z1 | Z1 |
| 45 | D45 | Z1 | Z1 | Z1 |
| 46 | D46 | Z1 | Z1 | Z1 |
| 47 | D47 | Z1 | Z1 | Z1 |
| 48 | D48 | Z1 | Z1 | Z1 |
| 49 | D49 | Z1 | Z1 | Z1 |
| 50 | D50 | Z1 | Z1 | Z1 |
| 51 | D51 | Z1 | Z1 | Z1 |
| 52 | D52 | Z1 | Z1 | Z1 |
| 53 | D53 | Z1 | Z1 | Z1 |
| 54 | D54 | Z1 | Z1 | Z1 |
| 55 | D55 | Z1 | Z1 | Z1 |
| 56 | D56 | Z1 | Z1 | Z1 |
| 57 | D57 | Z1 | Z1 | Z1 |
| 58 | D58 | Z1 | Z1 | Z1 |
| 59 | D59 | Z1 | Z1 | Z1 |
| 60 | D60 | Z1 | Z1 | Z1 |
| 61 | D61 | Z1 | Z1 | Z1 |
| 62 | D62 | Z1 | Z1 | Z1 |
| 63 | D63 | Z1 | Z1 | Z1 |
| 64 | D64 | Z1 | Z1 | Z1 |
| 65 | D65 | Z1 | Z1 | Z1 |
| 66 | D66 | Z1 | Z1 | Z1 |
| 67 | D67 | Z1 | Z1 | Z1 |
| 68 | D68 | Z1 | Z1 | Z1 |
| 69 | D69 | Z1 | Z1 | Z1 |
| 70 | D70 | Z1 | Z1 | Z1 |
| 71 | D71 | Z1 | Z1 | Z1 |
| 72 | D72 | Z1 | Z1 | Z1 |
| 73 | D73 | Z1 | Z1 | Z1 |
| 74 | D74 | Z1 | Z1 | Z1 |
| 75 | D75 | Z1 | Z1 | Z1 |
| 76 | D76 | Z1 | Z1 | Z1 |
| 77 | D77 | Z1 | Z1 | Z1 |
| 78 | D78 | Z1 | Z1 | Z1 |
| 79 | D79 | Z1 | Z1 | Z1 |
| 80 | D80 | Z1 | Z1 | Z1 |
| 81 | D81 | Z1 | Z1 | Z1 |
| 82 | D82 | Z1 | Z1 | Z1 |
| 83 | D83 | Z1 | Z1 | Z1 |
| 84 | D84 | Z1 | Z1 | Z1 |
| 85 | D85 | Z1 | Z1 | Z1 |
| 86 | D86 | Z1 | Z1 | Z1 |
| 87 | D87 | Z1 | Z1 | Z1 |
| 88 | D88 | Z1 | Z1 | Z1 |
| 89 | D89 | Z1 | Z1 | Z1 |
| 90 | D90 | Z1 | Z1 | Z1 |
| 91 | D91 | Z1 | Z1 | Z1 |
| 92 | D92 | Z1 | Z1 | Z1 |
| 93 | D93 | Z1 | Z1 | Z1 |
| 94 | D94 | Z1 | Z1 | Z1 |
| 95 | D95 | Z1 | Z1 | Z1 |
| 96 | D96 | Z1 | Z1 | Z1 |
| 97 | D97 | Z1 | Z1 | Z1 |
| 98 | D98 | Z1 | Z1 | Z1 |
| 99 | D99 | Z1 | Z1 | Z1 |
| 100 | D100 | Z1 | Z1 | Z1 |
| 101 | D101 | Z1 | Z1 | Z1 |
| 102 | D102 | Z1 | Z1 | Z1 |
| 103 | D103 | Z1 | Z1 | Z1 |
| 104 | D104 | Z1 | Z1 | Z1 |
| 105 | D105 | Z1 | Z1 | Z1 |
| 106 | D106 | Z1 | Z1 | Z1 |
| 107 | D107 | Z1 | Z1 | Z1 |
| 108 | D108 | Z1 | Z1 | Z1 |
| 109 | D109 | Z1 | Z1 | Z1 |
| 110 | D110 | Z1 | Z1 | Z1 |
| 111 | D111 | Z1 | Z1 | Z1 |
| 112 | D112 | Z1 | Z1 | Z1 |
| 113 | D113 | Z1 | Z1 | Z1 |
| 114 | D114 | Z1 | Z1 | Z1 |
| 115 | D115 | Z1 | Z1 | Z1 |
| 116 | D116 | Z1 | Z1 | Z1 |
| 117 | D117 | Z1 | Z1 | Z1 |
| 118 | D118 | Z1 | Z1 | Z1 |
| 119 | D119 | Z1 | Z1 | Z1 |
| 120 | D120 | Z1 | Z1 | Z1 |
| 121 | D121 | Z1 | Z1 | Z1 |
| 122 | D122 | Z1 | Z1 | Z1 |
| 123 | D123 | Z1 | Z1 | Z1 |
| 124 | D124 | Z1 | Z1 | Z1 |
| 125 | D125 | Z1 | Z1 | Z1 |
| 126 | D126 | Z1 | Z1 | Z1 |
| 127 | D127 | Z1 | Z1 | Z1 |
| 128 | D128 | Z1 | Z1 | Z1 |
| 129 | D129 | Z1 | Z1 | Z1 |
| 130 | D130 | Z1 | Z1 | Z1 |
| 131 | D131 | Z1 | Z1 | Z1 |
| 132 | D132 | Z1 | Z1 | Z1 |
| 133 | D133 | Z1 | Z1 | Z1 |
| 134 | D134 | Z1 | Z1 | Z1 |
| 135 | D135 | Z1 | Z1 | Z1 |
| 136 | D136 | Z1 | Z1 | Z1 |
| 137 | D137 | Z1 | Z1 | Z1 |
| 138 | D138 | Z1 | Z1 | Z1 |
| 139 | D139 | Z1 | Z1 | Z1 |
| 140 | D140 | Z1 | Z1 | Z1 |
| 141 | D141 | Z1 | Z1 | Z1 |
| 142 | D142 | Z1 | Z1 | Z1 |
| 143 | D143 | Z1 | Z1 | Z1 |
| 144 | D144 | Z1 | Z1 | Z1 |
| 145 | D145 | Z1 | Z1 | Z1 |
| 146 | D146 | Z1 | Z1 | Z1 |
| 147 | D147 | Z1 | Z1 | Z1 |
| 148 | D148 | Z1 | Z1 | Z1 |
| 149 | D149 | Z1 | Z1 | Z1 |
| 150 | D150 | Z1 | Z1 | Z1 |
| 151 | D151 | Z1 | Z1 | Z1 |
| 152 | D152 | Z1 | Z1 | Z1 |
| 153 | D153 | Z1 | Z1 | Z1 |
| 154 | D154 | Z1 | Z1 | Z1 |
| 155 | D155 | Z1 | Z1 | Z1 |
| 156 | D156 | Z1 | Z1 | Z1 |
| 157 | D157 | Z1 | Z1 | Z1 |
| 158 | D158 | Z1 | Z1 | Z1 |
| 159 | D159 | Z1 | Z1 | Z1 |
| 160 | D160 | Z1 | Z1 | Z1 |
| 161 | D161 | Z1 | Z1 | Z1 |
| 162 | D162 | Z1 | Z1 | Z1 |
| 163 | D163 | Z1 | Z1 | Z1 |
| 164 | D164 | Z1 | Z1 | Z1 |
| 165 | D165 | Z1 | Z1 | Z1 |
| 166 | D166 | Z1 | Z1 | Z1 |
| 167 | D167 | Z1 | Z1 | Z1 |
| 168 | D168 | Z1 | Z1 | Z1 |
| 169 | D169 | Z1 | Z1 | Z1 |
| 170 | D170 | Z1 | Z1 | Z1 |
| 171 | D171 | Z1 | Z1 | Z1 |
| 172 | D172 | Z1 | Z1 | Z1 |
| 173 | D173 | Z1 | Z1 | Z1 |
| 174 | D174 | Z1 | Z1 | Z1 |
| 175 | D175 | Z1 | Z1 | Z1 |
| 176 | D176 | Z1 | Z1 | Z1 |
| 177 | D177 | Z1 | Z1 | Z1 |
| 178 | D178 | Z1 | Z1 | Z1 |
| 179 | D179 | Z1 | Z1 | Z1 |
| 180 | D180 | Z1 | Z1 | Z1 |
| 181 | D181 | Z1 | Z1 | Z1 |
| 182 | D182 | Z1 | Z1 | Z1 |
| 183 | D183 | Z1 | Z1 | Z1 |
| 184 | D184 | Z1 | Z1 | Z1 |
| 185 | D185 | Z1 | Z1 | Z1 |
| 186 | D186 | Z1 | Z1 | Z1 |
| 187 | D187 | Z1 | Z1 | Z1 |
| 188 | D188 | Z1 | Z1 | Z1 |
| 189 | D189 | Z1 | Z1 | Z1 |
| 190 | D190 | Z1 | Z1 | Z1 |
| 191 | D191 | Z1 | Z1 | Z1 |
| 192 | D192 | Z1 | Z1 | Z1 |
| 193 | D193 | Z1 | Z1 | Z1 |
| 194 | D194 | Z1 | Z1 | Z1 |
| 195 | D195 | Z1 | Z1 | Z1 |
| 196 | D196 | Z1 | Z1 | Z1 |
| 197 | D197 | Z1 | Z1 | Z1 |
| 198 | D198 | Z1 | Z1 | Z1 |
| 199 | D199 | Z1 | Z1 | Z1 |
| 200 | D200 | Z1 | Z1 | Z1 |
| 201 | D201 | Z1 | Z1 | Z1 |
| 202 | D202 | Z1 | Z1 | Z1 |
| 203 | D203 | Z1 | Z1 | Z1 |
| 204 | D204 | Z1 | Z1 | Z1 |
| 205 | D205 | Z1 | Z1 | Z1 |
| 206 | D206 | Z1 | Z1 | Z1 |
| 207 | D207 | Z1 | Z1 | Z1 |
| 208 | D208 | Z1 | Z1 | Z1 |
| 209 | D209 | Z1 | Z1 | Z1 |
| 210 | D210 | Z1 | Z1 | Z1 |
| 211 | D211 | Z1 | Z1 | Z1 |
| 212 | D212 | Z1 | Z1 | Z1 |
| 213 | D213 | Z1 | Z1 | Z1 |
| 214 | D214 | Z1 | Z1 | Z1 |
| 215 | D215 | Z1 | Z1 | Z1 |
| 216 | D216 | Z1 | Z1 | Z1 |
| 217 | D217 | Z1 | Z1 | Z1 |
| 218 | D218 | Z1 | Z1 | Z1 |
| 219 | D219 | Z1 | Z1 | Z1 |
| 220 | D220 | Z1 | Z1 | Z1 |
| 221 | D221 | Z1 | Z1 | Z1 |
| 222 | D222 | Z1 | Z1 | Z1 |
| 223 | D223 | Z1 | Z1 | Z1 |
| 224 | D224 | Z1 | Z1 | Z1 |
| 225 | D225 | Z1 | Z1 | Z1 |
| 226 | D226 | Z1 | Z1 | Z1 |
| 227 | D227 | Z1 | Z1 | Z1 |
| 228 | D228 | Z1 | Z1 | Z1 |
| 229 | D229 | Z1 | Z1 | Z1 |
| 230 | D230 | Z1 | Z1 | Z1 |
| 231 | D231 | Z1 | Z1 | Z1 |
| 232 | D232 | Z1 | Z1 | Z1 |
| 233 | D233 | Z1 | Z1 | Z1 |
| 234 | D234 | Z1 | Z1 | Z1 |
| 235 | D235 | Z1 | Z1 | Z1 |
| 236 | D236 | Z1 | Z1 | Z1 |
| 237 | D237 | Z1 | Z1 | Z1 |
| 238 | D238 | Z1 | Z1 | Z1 |
| 239 | D239 | Z1 | Z1 | Z1 |
| 240 | D240 | Z1 | Z1 | Z1 |
| 241 | D241 | Z1 | Z1 | Z1 |
| 242 | D242 | Z1 | Z1 | Z1 |
| 243 | D243 | Z1 | Z1 | Z1 |
| 244 | D244 | Z1 | Z1 | Z1 |
| 245 | D245 | Z1 | Z1 | Z1 |
| 246 | D246 | Z1 | Z1 | Z1 |
| 247 | D247 | Z1 | Z1 | Z1 |
| 248 | D248 | Z1 | Z1 | Z1 |
| 249 | D249 | Z1 | Z1 | Z1 |
| 250 | D250 | Z1 | Z1 | Z1 |
| 251 | D251 | Z1 | Z1 | Z1 |
| 252 | D252 | Z1 | Z1 | Z1 |
| 253 | D253 | Z1 | Z1 | Z1 |
| 254 | D254 | Z1 | Z1 | Z1 |
| 255 | D255 | Z1 | Z1 | Z1 |
| 256 | D256 | Z1 | Z1 | Z1 |
| 257 | D257 | Z1 | Z1 | Z1 |
| 258 | D258 | Z1 | Z1 | Z1 |
| 259 | D259 | Z1 | Z1 | Z1 |
| 260 | D260 | Z1 | Z1 | Z1 |
| 261 | D261 | Z1 | Z1 | Z1 |
| 262 | D262 | Z1 | Z1 | Z1 |
| 263 | D263 | Z1 | Z1 | Z1 |
| 264 | D264 | Z1 | Z1 | Z1 |
| 265 | D265 | Z1 | Z1 | Z1 |
| 266 | D266 | Z1 | Z1 | Z1 |
| 267 | D267 | Z1 | Z1 | Z1 |
| 268 | D268 | Z1 | Z1 | Z1 |
| 269 | D269 | Z1 | Z1 | Z1 |
| 270 | D270 | Z1 | Z1 | Z1 |
| 271 | D271 | Z1 | Z1 | Z1 |
| 272 | D272 | Z1 | Z1 | Z1 |
| 273 | D273 | Z1 | Z1 | Z1 |
| 274 | D274 | Z1 | Z1 | Z1 |
| 275 | D275 | Z1 | Z1 | Z1 |
| 276 | D276 | Z1 | Z1 | Z1 |
| 277 | D277 | Z1 | Z1 | Z1 |
| 278 | D278 | Z1 | Z1 | Z1 |
| 279 | D279 | Z1 | Z1 | Z1 |
| 280 | D280 | Z1 | Z1 | Z1 |
| 281 | D281 | Z1 | Z1 | Z1 |
| 282 | D282 | Z1 | Z1 | Z1 |
| 283 | D283 | Z1 | Z1 | Z1 |
| 284 | D284 | Z1 | Z1 | Z1 |
| 285 | D285 | Z1 | Z1 | Z1 |
| 286 | D286 | Z1 | Z1 | Z1 |
| 287 | D287 | Z1 | Z1 | Z1 |
| 288 | D288 | Z1 | Z1 | Z1 |
| 289 | D289 | Z1 | Z1 | Z1 |
| 290 | D290 | Z1 | Z1 | Z1 |
| 291 | D291 | Z1 | Z1 | Z1 |
| 292 | D292 | Z1 | Z1 | Z1 |
| 293 | D293 | Z1 | Z1 | Z1 |
| 294 | D294 | Z1 | Z1 | Z1 |
| 295 | D295 | Z1 | Z1 | Z1 |
| 296 | D296 | Z1 | Z1 | Z1 |
| 297 | D297 | Z1 | Z1 | Z1 |
| 298 | D298 | Z1 | Z1 | Z1 |
| 299 | D299 | Z1 | Z1 | Z1 |
| 300 | D300 | Z1 | Z1 | Z1 |
| 301 | D301 | Z1 | Z1 | Z1 |
| 302 | D302 | Z1 | Z1 | Z1 |
| 303 | D303 | Z1 | Z1 | Z1 |
| 304 | D304 | Z1 | Z1 | Z1 |
| 305 | D305 | Z1 | Z1 | Z1 |
| 306 | D306 | Z1 | Z1 | Z1 |
| 307 | D307 | Z1 | Z1 | Z1 |
| 308 | D308 | Z1 | Z1 | Z1 |
| 309 | D309 | Z1 | Z1 | Z1 |
| 310 | D310 | Z1 | Z1 | Z1 |
| 311 | D311 | Z1 | Z1 | Z1 |
| 312 | D312 | Z1 | Z1 | Z1 |
| 313 | D313 | Z1 | Z1 | Z1 |
| 314 | D314 | Z1 | Z1 | Z1 |
| 315 | D315 | Z1 | Z1 | Z1 |
| 316 | D316 | Z1 | Z1 | Z1 |
| 317 | D317 | Z1 | Z1 | Z1 |
| 318 | D318 | Z1 | Z1 | Z1 |
| 319 | D319 | Z1 | Z1 | Z1 |
| 320 | D320 | Z1 | Z1 | Z1 |
| 321 | D321 | Z1 | Z1 | Z1 |
| 322 | D322 | Z1 | Z1 | Z1 |
| 323 | D323 | Z1 | Z1 | Z1 |
| 324 | D324 | Z1 | Z1 | Z1 |
| 325 | D325 | Z1 | Z1 | Z1 |
| 326 | D326 | Z1 | Z1 | Z1 |
| 327 | D327 | Z1 | Z1 | Z1 |
| 328 | D328 | Z1 | Z1 | Z1 |
| 329 | D329 | Z1 | Z1 | Z1 |
| 330 | D330 | Z1 | Z1 | Z1 |
| 331 | D331 | Z1 | Z1 | Z1 |
| 332 | D332 | Z1 | Z1 | Z1 |
| 333 | D333 | Z1 | Z1 | Z1 |
| 334 | D334 | Z1 | Z1 | Z1 |
| 335 | D335 | Z1 | Z1 | Z1 |
| 336 | D336 | Z1 | Z1 | Z1 |
| 337 | D337 | Z1 | Z1 | Z1 |
| 338 | D338 | Z1 | Z1 | Z1 |
| 339 | D339 | Z1 | Z1 | Z1 |
| 340 | D340 | Z1 | Z1 | Z1 |
| 341 | D341 | Z1 | Z1 | Z1 |
| 342 | D342 | Z1 | Z1 | Z1 |
| 343 | D343 | Z1 | Z1 | Z1 |
| 344 | D344 | Z1 | Z1 | Z1 |
| 345 | D345 | Z1 | Z1 | Z1 |
| 346 | D346 | Z1 | Z1 | Z1 |
| 347 | D347 | Z1 | Z1 | Z1 |
| 348 | D348 | Z1 | Z1 | Z1 |
| 349 | D349 | Z1 | Z1 | Z1 |
| 350 | D350 | Z1 | Z1 | Z1 |
| 351 | D351 | Z1 | Z1 | Z1 |
| 352 | D352 | Z1 | Z1 | Z1 |
| 353 | D353 | Z1 | Z1 | Z1 |
| 354 | D354 | Z1 | Z1 | Z1 |
| 355 | D355 | Z1 | Z1 | Z1 |
| 356 | D356 | Z1 | Z1 | Z1 |
| 357 | D357 | Z1 | Z1 | Z1 |
| 358 | D358 | Z1 | Z1 | Z1 |
| 359 | D359 | Z1 | Z1 | Z1 |
| 360 | D360 | Z1 | Z1 | Z1 |
| 361 | D361 | Z1 | Z1 | Z1 |
| 362 | D362 | Z1 | Z1 | Z1 |
| 363 | D363 | Z1 | Z1 | Z1 |
| 364 | D364 | Z1 | Z1 | Z1 |
| 365 | D365 | Z1 | Z1 | Z1 |
| 366 | D366 | Z1 | Z1 | Z1 |
| 367 | D367 | Z1 | Z1 | Z1 |
| 368 | D368 | Z1 | Z1 | Z1 |
| 369 | D369 | Z1 | Z1 | Z1 |
| 370 | D370 | Z1 | Z1 | Z1 |
| 371 | D371 | Z1 | Z1 | Z1 |
| 372 | D372 | Z1 | Z1 | Z1 |
| 373 | D373 | Z1 | Z1 | Z1 |
| 374 | D374 | Z1 | Z1 | Z1 |
| 375 | D375 | Z1 | Z1 | Z1 |
| 376 | D376 | Z1 | Z1 | Z1 |
| 377 | D377 | Z1 | Z1 | Z1 |
| 378 | D378 | Z1 | Z1 | Z1 |
| 379 | D379 | Z1 | Z1 | Z1 |
| 380 | D380 | Z1 | Z1 | Z1 |
| 381 | D381 | Z1 | Z1 | Z1 |
| 382 | D382 | Z1 | Z1 | Z1 |
| 383 | D383 | Z1 | Z1 | Z1 |
| 384 | D384 | Z1 | Z1 | Z1 |
| 385 | D385 | Z1 | Z1 | Z1 |
| 386 | D386 | Z1 | Z1 | Z1 |
| 387 | D387 | Z1 | Z1 | Z1 |
| 388 | D388 | Z1 | Z1 | Z1 |
| 389 | D389 | Z1 | Z1 | Z1 |
| 390 | D390 | Z1 | Z1 | Z1 |
| 391 | D391 | Z1 | Z1 | Z1 |
| 392 | D392 | Z1 | Z1 | Z1 |
| 393 | D393 | Z1 | Z1 | Z1 |
| 394 | D394 | Z1 | Z1 | Z1 |
| 395 | D395 | Z1 | Z1 | Z1 |
| 396 | D396 | Z1 | Z1 | Z1 |
| 397 | D397 | Z1 | Z1 | Z1 |
| 398 | D398 | Z1 | Z1 | Z1 |
| 399 | D399 | Z1 | Z1 | Z1 |
| 400 | D400 | Z1 | Z1 | Z1 |
| 401 | D401 | Z1 | Z1 | Z1 |
| 402 | D402 | Z1 | Z1 | Z1 |
| 403 | D403 | Z1 | Z1 | Z1 |
| 404 | D404 | Z1 | Z1 | Z1 |
| 405 | D405 | Z1 | Z1 | Z1 |
| 406 | D406 | Z1 | Z1 | Z1 |
| 407 | D407 | Z1 | Z1 | Z1 |
| 408 | D408 | Z1 | Z1 | Z1 |
| 409 | D409 | Z1 | Z1 | Z1 |
| 410 | D410 | Z1 | Z1 | Z1 |
| 411 | D411 | Z1 | Z1 | Z1 |
| 412 | D412 | Z1 | Z1 | Z1 |
| 413 | D413 | Z1 | Z1 | Z1 |
| 414 | D414 | Z1 | Z1 | Z1 |
| 415 | D415 | Z1 | Z1 | Z1 |
| 416 | D416 | Z1 | Z1 | Z1 |
| 417 | D417 | Z1 | Z1 | Z1 |
| 418 | D418 | Z1 | Z1 | Z1 |
| 419 | D419 | Z1 | Z1 | Z1 |
| 420 | D420 | Z1 | Z1 | Z1 |
| 421 | D421 | Z1 | Z1 | Z1 |
| 422 | D422 | Z1 | Z1 | Z1 |
| 423 | D423 | Z1 | Z1 | Z1 |
| 424 | D424 | Z1 | Z1 | Z1 |
| 425 | D425 | Z1 | Z1 | Z1 |
| 426 | D426 | Z1 | Z1 | Z1 |
| 427 | D427 | Z1 | Z1 | Z1 |
| 428 | D428 | Z1 | Z1 | Z1 |
| 429 | D429 | Z1 | Z1 | Z1 |
| 430 | D430 | Z1 | Z1 | Z1 |
| 431 | D431 | Z1 | Z1 | Z1 |
| 432 | D432 | Z1 | Z1 | Z1 |
| 433 | D433 | Z1 | Z1 | Z1 |
| 434 | D434 | Z1 | Z1 | Z1 |
| 435 | D435 | Z1 | Z1 | Z1 |
| 436 | D436 | Z1 | Z1 | Z1 |
| 437 | D437 | Z1 | Z1 | Z1 |
| 438 | D438 | Z1 | Z1 | Z1 |
| 439 | D439 | Z1 | Z1 | Z1 |
| 440 | D440 | Z1 | Z1 | Z1 |
| 441 | D441 | Z1 | Z1 | Z1 |
| 442 | D442 | Z1 | Z1 | Z1 |
| 443 | D443 | Z1 | Z1 | Z1 |
| 444 | D444 | Z1 | Z1 | Z1 |
| 445 | D445 | Z1 | Z1 | Z1 |
| 446 | D446 | Z1 | Z1 | Z1 |
| 447 | D447 | Z1 | Z1 | Z1 |
| 448 | D448 | Z1 | Z1 | Z1 |
| 449 | D449 | Z1 | Z1 | Z1 |
| 450 | D450 | Z1 | Z1 | Z1 |
| 451 | D451 | Z1 | Z1 | Z1 |
| 452 | D452 | Z1 | Z1 | Z1 |
| 453 | D453 | Z1 | Z1 | Z1 |
| 454 | D454 | Z1 | Z1 | Z1 |
| 455 | D455 | Z1 | Z1 | Z1 |
| 456 | D456 | Z1 | Z1 | Z1 |
| 457 | D457 | Z1 | Z1 | Z1 |
| 458 | D458 | Z1 | Z1 | Z1 |
| 459 | D459 | Z1 | Z1 | Z1 |
| 460 | D460 | Z1 | Z1 | Z1 |
| 461 | D461 | Z1 | Z1 | Z1 |
| 462 | D462 | Z1 | Z1 | Z1 |
| 463 | D463 | Z1 | Z1 | Z1 |
| 464 | D464 | Z1 | Z1 | Z1 |
| 465 | D465 | Z1 | Z1 | Z1 |
| 466 | D466 | Z1 | Z1 | Z1 |
| 467 | D467 | Z1 | Z1 | Z1 |
| 468 | D468 | Z1 | Z1 | Z1 |
| 469 | D469 | Z1 | Z1 | Z1 |
| 470 | D470 | Z1 | Z1 | Z1 |
| 471 | D471 | Z1 | Z1 | Z1 |
| 472 | D472 | Z1 | Z1 | Z1 |
| 473 | D473 | Z1 | Z1 | Z1 |
| 474 | D474 | Z1 | Z1 | Z1 |
| 475 | D475 | Z1 | Z1 | Z1 |
| 476 | D476 | Z1 | Z1 | Z1 |
| 477 | D477 | Z1 | Z1 | Z1 |
| 478 | D478 | Z1 | Z1 | Z1 |
| 479 | D479 | Z1 | Z1 | Z1 |
| 480 | D480 | Z1 | Z1 | Z1 |
| 481 | D481 | Z1 | Z1 | Z1 |
| 482 | D482 | Z1 | Z1 | Z1 |
| 483 | D483 | Z1 | Z1 | Z1 |
| 484 | D484 | Z1 | Z1 | Z1 |
| 485 | D485 | Z1 | Z1 | Z1 |
| 486 | D486 | Z1 | Z1 | Z1 |
| 487 | D487 | Z1 | Z1 | Z1 |
| 488 | D488 | Z1 | Z1 | Z1 |
| 489 | D489 | Z1 | Z1 | Z1 |
| 490 | D490 | Z1 | Z1 | Z1 |
| 491 | D491 | Z1 | Z1 | Z1 |
| 492 | D492 | Z1 | Z1 | Z1 |
| 493 | D493 | Z1 | Z1 | Z1 |
| 494 | D494 | Z1 | Z1 | Z1 |
| 495 | D495 | Z1 | Z1 | Z1 |
| 496 | D496 | Z1 | Z1 | Z1 |
| 497 | D497 | Z1 | Z1 | Z1 |
| 498 | D498 | Z1 | Z1 | Z1 |
| 499 | D499 | Z1 | Z1 | Z1 |
| 500 | D500 | Z1 | Z1 | Z1 |
| 501 | D501 | Z1 | Z1 | Z1 |
| 502 | D502 | Z1 | Z1 | Z1 |
| 503 | D503 | Z1 | Z1 | Z1 |
| 504 | D504 | Z1 | Z1 | Z1 |
| 505 | D505 | Z1 | Z1 | Z1 |
| 506 | D506 | Z1 | Z1 | Z1 |
| 507 | D507 | Z1 | Z1 | Z1 |
| 508 | D508 | Z1 | Z1 | Z1 |
| 509 | D509 | Z1 | Z1 | Z1 |
| 510 | D510 | Z1 | Z1 | Z1 |
| 511 | D511 | Z1 | Z1 | Z1 |
| 512 | D512 | Z1 | Z1 | Z1 |
| 513 | D513 | Z1 | Z1 | Z1 |
| 514 | D514 | Z1 | Z1 | Z1 |
| 515 | D515 | Z1 | Z1 | Z1 |
| 516 | D516 | Z1 | Z1 | Z1 |
| 517 | D517 | Z1 | Z1 | Z1 |
| 518 | D518 | Z1 | Z1 | Z1 |
| 519 | D519 | Z1 | Z1 | Z1 |
| 520 | D520 | Z1 | Z1 | Z1 |
| 521 | D521 | Z1 | Z1 | Z1 |
| 522 | D522 | Z1 | Z1 | Z1 |
| 523 | D523 | Z1 | Z1 | Z1 |
| 524 | D524 | Z1 | Z1 | Z1 |
| 525 | D525 | Z1 | Z1 | Z1 |
| 526 | D526 | Z1 | Z1 | Z1 |
| 527 | D527 | Z1 | Z1 | Z1 |
| 528 | D528 | Z1 | Z1 | Z1 |
| 529 | D529 | Z1 | Z1 | Z1 |
| 530 | D530 | Z1 | Z1 | Z1 |
| 531 | D531 | Z1 | Z1 | Z1 |
| 532 | D532 | Z1 | Z1 | Z1 |
| 533 | D533 | Z1 | Z1 | Z1 |
| 534 | D534 | Z1 | Z1 | Z1 |
| 535 | D535 | Z1 | Z1 | Z1 |
| 536 | D536 | Z1 | Z1 | Z1 |
| 537 | D537 | Z1 | Z1 | Z1 |
| 538 | D538 | Z1 | Z1 | Z1 |
| 539 | D539 | Z1 | Z1 | Z1 |
| 540 | D540 | Z1 | Z1 | Z1 |
| 541 | D541 | Z1 | Z1 | Z1 |
| 542 | D542 | Z1 | Z1 | Z1 |
| 543 | D543 | Z1 | Z1 | Z1 |
| 544 | D544 | Z1 | Z1 | Z1 |
| 545 | D545 | Z1 | Z1 | Z1 |
| 546 | D546 | Z1 | Z1 | Z1 |
| 547 | D547 | Z1 | Z1 | Z1 |
| 548 | D548 | Z1 | Z1 | Z1 |
| 549 | D549 | Z1 | Z1 | Z1 |
| 550 | D550 | Z1 | Z1 | Z1 |
| 551 | D551 | Z1 | Z1 | Z1 |
| 552 | D552 | Z1 | Z1 | Z1 |
| 553 | D553 | Z1 | Z1 | Z1 |
| 554 | D554 | Z1 | Z1 | Z1 |
| 555 | D555 | Z1 | Z1 | Z1 |
| 556 | D556 | Z1 | Z1 | Z1 |
| 557 | D557 | Z1 | Z1 | Z1 |
| 558 | D558 | Z1 | Z1 | Z1 |
| 559 | D559 | Z1 | Z1 | Z1 |
| 560 | D560 | Z1 | Z1 | Z1 |
| 561 | D561 | Z1 | Z1 | Z1 |
| 562 | D562 | Z1 | Z1 | Z1 |
| 563 | D563 | Z1 | Z1 | Z1 |
| 564 | D564 | Z1 | Z1 | Z1 |
| 565 | D565 | Z1 | Z1 | Z1 |
| 566 | D566 | Z1 | Z1 | Z1 |
| 567 | D567 | Z1 | Z1 | Z1 |
| 568 | D568 | Z1 | Z1 | Z1 |
| 569 | D569 | Z1 | Z1 | Z1 |
| 570 | D570 | Z1 | Z1 | Z1 |
| 571 | D571 | Z1 | Z1 | Z1 |
| 572 | D572 | Z1 | Z1 | Z1 |
| 573 | D573 | Z1 | Z1 | Z1 |
| 574 | D574 | Z1 | Z1 | Z1 |
| 575 | D575 | Z1 | Z1 | Z1 |
| 576 | D576 | Z1 | Z1 | Z1 |
| 577 | D577 | Z1 | Z1 | Z1 |
| 578 | D578 | Z1 | Z1 | Z1 |
| 579 | D579 | Z1 | Z1 | Z1 |
| 580 | D580 | Z1 | Z1 | Z1 |
| 581 | D581 | Z1 | Z1 | Z1 |
| 582 | D582 | Z1 | Z1 | Z1 |
| 583 | D583 | Z1 | Z1 | Z1 |
| 584 | D584 | Z1 | Z1 | Z1 |
| 585 | D585 | Z1 | Z1 | Z1 |
| 586 | D586 | Z1 | Z1 | Z1 |
| 587 | D587 | Z1 | Z1 | Z1 |
| 588 | D588 | Z1 | Z1 | Z1 |
| 589 | D589 | Z1 | Z1 | Z1 |
| 590 | D590 | Z1 | Z1 | Z1 |
| 591 | D591 | Z1 | Z1 | Z1 |
| 592 | D592 | Z1 | Z1 | Z1 |
| 593 | D593 | Z1 | Z1 | Z1 |
| 594 | D594 | Z1 | Z1 | Z1 |
| 595 | D595 | Z1 | Z1 | Z1 |
| 596 | D596 | Z1 | Z1 | Z1 |
| 597 | D597 | Z1 | Z1 | Z1 |
| 598 | D598 | Z1 | Z1 | Z1 |
| 599 | D599 | Z1 | Z1 | Z1 |
| 600 | D600 | Z1 | Z1 | Z1 |
| 601 | D601 | Z1 | Z1 | Z1 |
| 602 | D602 | Z1 | Z1 | Z1 |
| 603 | D603 | Z1 | Z1 | Z1 |
| 604 | D604 | Z1 | Z1 | Z1 |
| 605 | D605 | Z1 | Z1 | Z1 |
| 606 | D606 | Z1 | Z1 | Z1 |
| 607 | D607 | Z1 | Z1 | Z1 |
| 608 | D608 | Z1 | Z1 | Z1 |
| 609 | D609 | Z1 | Z1 | Z1 |
| 610 | D610 | Z1 | Z1 | Z1 |
| 611 | D611 | Z1 | Z1 | Z1 |
| 612 | D612 | Z1 | Z1 | Z1 |
| 613 | D613 | Z1 | Z1 | Z1 |
| 614 | D614 | Z1 | Z1 | Z1 |
| 615 | D615 | Z1 | Z1 | Z1 |
| 616 | D616 | Z1 | Z1 | Z1 |
| 617 | D617 | Z1 | Z1 | Z1 |
| 618 | D618 | Z1 | Z1 | Z1 |
| 619 | D619 | Z1 | Z1 | Z1 |
| 620 | D620 | Z1 | Z1 | Z1 |
| 621 | D621 | Z1 | Z1 | Z1 |
| 622 | D622 | Z1 | Z1 | Z1 |
| 623 | D623 | Z1 | Z1 | Z1 |
| 624 | D624 | Z1 | Z1 | Z1 |
| 625 | D625 | Z1 | Z1 | Z1 |
| 626 | D626 | Z1 | Z1 | Z1 |
| 627 | D627 | Z1 | Z1 | Z1 |
| 628 | D628 | Z1 | Z1 | Z1 |
| 629 | D629 | Z1 | Z1 | Z1 |
| 630 | D630 | Z1 | Z1 | Z1 |
| 631 | D631 | Z1 | Z1 | Z1 |
| 632 | D632 | Z1 | Z1 | Z1 |
| 633 | D633 | Z1 | Z1 | Z1 |
| 634 | D634 | Z1 | Z1 | Z1 |
| 635 | D635 | Z1 | Z1 | Z1 |
| 636 | D636 | Z1 | Z1 | Z1 |
| 637 | D637 | Z1 | Z1 | Z1 |
| 638 | D638 | Z1 | Z1 | Z1 |
| 639 | D639 | Z1 | Z1 | Z1 |
| 640 | D640 | Z1 | Z1 | Z1 |
| 641 | D641 | Z1 | Z1 | Z1 |
| 642 | D642 | Z1 | Z1 | Z1 |
| 643 | D643 | Z1 | Z1 | Z1 |
| 644 | D644 | Z1 | Z1 | Z1 |
| 645 | D645 | Z1 | Z1 | Z1 |
| 646 | D646 | Z1 | Z1 | Z1 |
| 647 | D647 | Z1 | Z1 | Z1 |
| 648 | D648 | Z1 | Z1 | Z1 |
| 649 | D649 | Z1 | Z1 | Z1 |
| 650 | D650 | Z1 | Z1 | Z1 |
| 651 | D651 | Z1 | Z1 | Z1 |
| 652 | D652 | Z1 | Z1 | Z1 |
| 653 | D653 | Z1 | Z1 | Z1 |
| 654 | D654 | Z1 | Z1 | Z1 |
| 655 | D655 | Z1 | Z1 | Z1 |
| 656 | D656 | Z1 | Z1 | Z1 |
| 657 | D657 | Z1 | Z1 | Z1 |
| 658 | D658 | Z1 | Z1 | Z1 |
| 659 | D659 | Z1 | Z1 | Z1 |
| 660 | D660 | Z1 | Z1 | Z1 |
| 661 | D661 | Z1 | Z1 | Z1 |
| 662 | D662 | Z1 | Z1 | Z1 |
| 663 | D663 | Z1 | Z1 | Z1 |
| 664 | D664 | Z1 | Z1 | Z1 |
| 665 | D665 | Z1 | Z1 | Z1 |
| 666 | D666 | Z1 | Z1 | Z1 |
| 667 | D667 | Z1 | Z1 | Z1 |
| 668 | D668 | Z1 | Z1 | Z1 |
| 669 | D669 | Z1 | Z1 | Z1 |
| 670 | D670 | Z1 | Z1 | Z1 |
| 671 | D671 | Z1 | Z1 | Z1 |
| 672 | D672 | Z1 | Z1 | Z1 |
| 673 | D673 | Z1 | Z1 | Z1 |
| 674 | D674 | Z1 | Z1 | Z1 |
| 675 | D675 | Z1 | Z1 | Z1 |
| 676 | D676 | Z1 | Z1 | Z1 |
| 677 | D677 | Z1 | Z1 | Z1 |
| 678 | D678 | Z1 | Z1 | Z1 |
| 679 | D679 | Z1 | Z1 | Z1 |
| 680 | D680 | Z1 | Z1 | Z1 |
| 681 | D681 | Z1 | Z1 | Z1 |
| 682 | D682 | Z1 | Z1 | Z1 |
| 683 | D683 | Z1 | Z1 | Z1 |
| 684 | D684 | Z1 | Z1 | Z1 |
| 685 | D685 | Z1 | Z1 | Z1 |
| 686 | D686 | Z1 | Z1 | Z1 |
| 687 | D687 | Z1 | Z1 | Z1 |
| 688 | D688 | Z1 | Z1 | Z1 |
| 689 | D689 | Z1 | Z1 | Z1 |
| 690 | D690 | Z1 | Z1 | Z1 |
| 691 | D691 | Z1 | Z1 | Z1 |
| 692 | D692 | Z1 | Z1 | Z1 |
| 693 | D693 | Z1 | Z1 | Z1 |
| 694 | D694 | Z1 | Z1 | Z1 |
| 695 | D695 | Z1 | Z1 | Z1 |
| 696 | D696 | Z1 | Z1 | Z1 |
| 697 | D697 | Z1 | Z1 | Z1 |
| 698 | D698 | Z1 | Z1 | Z1 |
| 699 | D699 | Z1 | Z1 | Z1 |
| 700 | D700 | Z1 | Z1 | Z1 |
| 701 | D701 | Z1 | Z1 | Z1 |
| 702 | D702 | Z1 | Z1 | Z1 |
| 703 | D703 | Z1 | Z1 | Z1 |
| 704 | D704 | Z1 | Z1 | Z1 |
| 705 | D705 | Z1 | Z1 | Z1 |
| 706 | D706 | Z1 | Z1 | Z1 |
| 707 | D707 | Z1 | Z1 | Z1 |
| 708 | D708 | Z1 | Z1 | Z1 |
| 709 | D709 | Z1 | Z1 | Z1 |
| 710 | D710 | Z1 | Z1 | Z1 |
| 711 | D711 | Z1 | Z1 | Z1 |
| 712 | D712 | Z1 | Z1 | Z1 |
| 713 | D713 | Z1 | Z1 | Z1 |
| 714 | D714 | Z1 | Z1 | Z1 |
| 715 | D715 | Z1 | Z1 | Z1 |
| 716 | D716 | Z1 | Z1 | Z1 |
| 717 | D717 | Z1 | Z1 | Z1 |
| 718 | D718 | Z1 | Z1 | Z1 |
| 719 | D719 | Z1 | Z1 | Z1 |
| 720 | D720 | Z1 | Z1 | Z1 |
| 721 | D721 | Z1 | Z1 | Z1 |
| 722 | D722 | Z1 | Z1 | Z1 |
| 723 | D723 | Z1 | Z1 | Z1 |
| 724 | D724 | Z1 | Z1 | Z1 |
| 725 | D725 | Z1 | Z1 | Z1 |
| 726 | D726 | Z1 | Z1 | Z1 |
| 727 | D727 | Z1 | Z1 | Z1 |
| 728 | D728 | Z1 | Z1 | Z1 |
| 729 | D729 | Z1 | Z1 | Z1 |
| 730 | 0730 | Z1 | Z1 | Z1 |
| 731 | D731 | Z1 | Z1 | Z1 |
| 732 | D732 | Z1 | Z1 | Z1 |
| 733 | D733 | Z1 | Z1 | Z1 |
| 734 | D734 | Z1 | Z1 | Z1 |
| 735 | D735 | Z1 | Z1 | Z1 |
| 736 | D736 | Z1 | Z1 | Z1 |
| 737 | D737 | Z1 | Z1 | Z1 |
| 738 | D738 | Z1 | Z1 | Z1 |
| 739 | D739 | Z1 | Z1 | Z1 |
| 740 | D740 | Z1 | Z1 | Z1 |
| 741 | D741 | Z1 | Z1 | Z1 |
| 742 | D742 | Z1 | Z1 | Z1 |
| 743 | D743 | Z1 | Z1 | Z1 |
| 744 | D744 | Z1 | Z1 | Z1 |
| 745 | D745 | Z1 | Z1 | Z1 |
| 746 | D746 | Z1 | Z1 | Z1 |
| 747 | D747 | Z1 | Z1 | Z1 |
| 748 | D748 | Z1 | Z1 | Z1 |
| 749 | D749 | Z1 | Z1 | Z1 |
| 750 | D750 | Z1 | Z1 | Z1 |
| 751 | D751 | Z1 | Z1 | Z1 |
| 752 | D752 | Z1 | Z1 | Z1 |
| 753 | D753 | Z1 | Z1 | Z1 |
| 754 | D754 | Z1 | Z1 | Z1 |
| 755 | D755 | Z1 | Z1 | Z1 |
| 756 | D756 | Z1 | Z1 | Z1 |
| 757 | D757 | Z1 | Z1 | Z1 |
| 758 | D758 | Z1 | Z1 | Z1 |
| 759 | D759 | Z1 | Z1 | Z1 |
| 760 | D760 | Z1 | Z1 | Z1 |
| 761 | D761 | Z1 | Z1 | Z1 |
| 762 | D762 | Z1 | Z1 | Z1 |
| 763 | D763 | Z1 | Z1 | Z1 |
| 764 | D764 | Z1 | Z1 | Z1 |
| 765 | D765 | Z1 | Z1 | Z1 |
| 766 | D766 | Z1 | Z1 | Z1 |
| 767 | D767 | Z1 | Z1 | Z1 |
| 768 | D768 | Z1 | Z1 | Z1 |
| 769 | D769 | Z1 | Z1 | Z1 |
| 770 | D770 | Z1 | Z1 | Z1 |
| 771 | D771 | Z1 | Z1 | Z1 |
| 772 | D772 | Z1 | Z1 | Z1 |
| 773 | D773 | Z1 | Z1 | Z1 |
| 774 | D774 | Z1 | Z1 | Z1 |
| 775 | D775 | Z1 | Z1 | Z1 |
| 776 | D776 | Z1 | Z1 | Z1 |
| 777 | D777 | Z1 | Z1 | Z1 |
| 778 | D778 | Z1 | Z1 | Z1 |
| 779 | D779 | Z1 | Z1 | Z1 |
| 780 | D780 | Z1 | Z1 | Z1 |
| 781 | D781 | Z1 | Z1 | Z1 |
| 782 | D782 | Z1 | Z1 | Z1 |
| 783 | D783 | Z1 | Z1 | Z1 |
| 784 | D784 | Z1 | Z1 | Z1 |
| 785 | D785 | Z1 | Z1 | Z1 |
| 786 | D786 | Z1 | Z1 | Z1 |
| 787 | D787 | Z1 | Z1 | Z1 |
| 788 | D788 | Z1 | Z1 | Z1 |
| 789 | D789 | Z1 | Z1 | Z1 |
| 790 | D790 | Z1 | Z1 | Z1 |
| 791 | D791 | Z1 | Z1 | Z1 |
| 792 | D792 | Z1 | Z1 | Z1 |
| 793 | D793 | Z1 | Z1 | Z1 |
| 794 | D794 | Z1 | Z1 | Z1 |
| 795 | D795 | Z1 | Z1 | Z1 |
| 796 | D796 | Z1 | Z1 | Z1 |
| 797 | D797 | Z1 | Z1 | Z1 |
| 798 | D798 | Z1 | Z1 | Z1 |
| 799 | D799 | Z1 | Z1 | Z1 |
| 800 | D800 | Z1 | Z1 | Z1 |
| 801 | D801 | Z1 | Z1 | Z1 |
| 802 | D802 | Z1 | Z1 | Z1 |
| 803 | D803 | Z1 | Z1 | Z1 |
| 804 | D804 | Z1 | Z1 | Z1 |
| 805 | D805 | Z1 | Z1 | Z1 |
| 806 | D806 | Z1 | Z1 | Z1 |
| 807 | D807 | Z1 | Z1 | Z1 |
| 808 | D808 | Z1 | Z1 | Z1 |
| 809 | D809 | Z1 | Z1 | Z1 |
| 810 | D810 | Z1 | Z1 | Z1 |
| 811 | D811 | Z1 | Z1 | Z1 |
| 812 | D812 | Z1 | Z1 | Z1 |
| 813 | D813 | Z1 | Z1 | Z1 |
| 814 | D814 | Z1 | Z1 | Z1 |
| 815 | D815 | Z1 | Z1 | Z1 |
| 816 | D816 | Z1 | Z1 | Z1 |
| 817 | D817 | Z1 | Z1 | Z1 |
| 818 | D818 | Z1 | Z1 | Z1 |
| 819 | D819 | Z1 | Z1 | Z1 |
| 820 | D820 | Z1 | Z1 | Z1 |
| 821 | D821 | Z1 | Z1 | Z1 |
| 822 | D822 | Z1 | Z1 | Z1 |
| 823 | D823 | Z1 | Z1 | Z1 |
| 824 | D824 | Z1 | Z1 | Z1 |
| 825 | D825 | Z1 | Z1 | Z1 |
| 826 | D826 | Z1 | Z1 | Z1 |
| 827 | D827 | Z1 | Z1 | Z1 |
| 828 | D828 | Z1 | Z1 | Z1 |
| 829 | D829 | Z1 | Z1 | Z1 |
| 830 | D830 | Z1 | Z1 | Z1 |
| 831 | D831 | Z1 | Z1 | Z1 |
| 832 | D832 | Z1 | Z1 | Z1 |
| 833 | D833 | Z1 | Z1 | Z1 |
| 834 | D834 | Z1 | Z1 | Z1 |
| 835 | D835 | Z1 | Z1 | Z1 |
| 836 | D836 | Z1 | Z1 | Z1 |
| 837 | D837 | Z1 | Z1 | Z1 |
| 838 | D838 | Z1 | Z1 | Z1 |
| 839 | D839 | Z1 | Z1 | Z1 |
| 840 | D840 | Z1 | Z1 | Z1 |
| 841 | D841 | Z1 | Z1 | Z1 |
| 842 | D842 | Z1 | Z1 | Z1 |
| 843 | D843 | Z1 | Z1 | Z1 |
| 844 | D844 | Z1 | Z1 | Z1 |
| 845 | D845 | Z1 | Z1 | Z1 |
| 846 | D846 | Z1 | Z1 | Z1 |
| 847 | D847 | Z1 | Z1 | Z1 |
| 848 | D848 | Z1 | Z1 | Z1 |
| 849 | D849 | Z1 | Z1 | Z1 |
| 850 | D850 | Z1 | Z1 | Z1 |
| 851 | D851 | Z1 | Z1 | Z1 |
| 852 | D852 | Z1 | Z1 | Z1 |
| 853 | D853 | Z1 | Z1 | Z1 |
| 854 | D854 | Z1 | Z1 | Z1 |
| 855 | D855 | Z1 | Z1 | Z1 |
| 856 | D856 | Z1 | Z1 | Z1 |
| 857 | D857 | Z1 | Z1 | Z1 |
| 858 | D858 | Z1 | Z1 | Z1 |
| 859 | D859 | Z1 | Z1 | Z1 |
| 860 | D860 | Z1 | Z1 | Z1 |
| 861 | D861 | Z1 | Z1 | Z1 |
| 862 | D862 | Z1 | Z1 | Z1 |
| 863 | D863 | Z1 | Z1 | Z1 |
| 864 | D864 | Z1 | Z1 | Z1 |
| 865 | D865 | Z1 | Z1 | Z1 |
| 866 | D866 | Z1 | Z1 | Z1 |
| 867 | D867 | Z1 | Z1 | Z1 |
| 868 | D868 | Z1 | Z1 | Z1 |
| 869 | D869 | Z1 | Z1 | Z1 |
| 870 | D870 | Z1 | Z1 | Z1 |
| 871 | D871 | Z1 | Z1 | Z1 |
| 872 | D872 | Z1 | Z1 | Z1 |
| 873 | D873 | Z1 | Z1 | Z1 |
| 874 | D874 | Z1 | Z1 | Z1 |
| 875 | D875 | Z1 | Z1 | Z1 |
| 876 | D876 | Z1 | Z1 | Z1 |
| 877 | D877 | Z1 | Z1 | Z1 |
| 878 | D878 | Z1 | Z1 | Z1 |
| 879 | D879 | Z1 | Z1 | Z1 |
| 880 | D880 | Z1 | Z1 | Z1 |
| 881 | D881 | Z1 | Z1 | Z1 |
| 882 | D882 | Z1 | Z1 | Z1 |
| 883 | D883 | Z1 | Z1 | Z1 |
| 884 | D884 | Z1 | Z1 | Z1 |
| 885 | D885 | Z1 | Z1 | Z1 |
| 886 | D886 | Z1 | Z1 | Z1 |
| 887 | D887 | Z1 | Z1 | Z1 |
| 888 | D888 | Z1 | Z1 | Z1 |

TABLE 2

| No. | R¹ | R² | R³ | R⁴ |
|---|---|---|---|---|
| 1~888 | D1~D888 | Z1 | Z1 | Z1 |
| 889~1776 | D1~D888 | Z2 | Z2 | Z2 |
| 1777~2664 | D1~D888 | Z3 | Z3 | Z3 |
| 2665~3552 | D1~D888 | Z4 | Z4 | Z4 |
| 3553~4440 | D1~D888 | Z5 | Z5 | Z5 |
| 4441~5328 | D1~D888 | Z6 | Z6 | Z6 |
| 5329~6216 | D1~D888 | Z7 | Z7 | Z7 |
| 6217~7104 | D1~D888 | Z8 | Z8 | Z8 |
| 7105~7992 | D1~D888 | Z9 | Z9 | Z9 |
| 7993~8880 | D1~D888 | Z10 | Z10 | Z10 |
| 8881~9768 | D1~D888 | Z11 | Z11 | Z11 |
| 9769~10656 | D1~D888 | Z12 | Z12 | Z12 |
| 10657~11544 | D1~D888 | Z13 | Z13 | Z13 |
| 11545~12432 | D1~D888 | Z14 | Z14 | Z14 |
| 12433~13320 | D1~D888 | Z15 | Z15 | Z15 |
| 13321~14208 | D1~D888 | Z16 | Z16 | Z16 |
| 14209~15096 | D1~D888 | Z17 | Z17 | Z17 |
| 15097~15984 | D1~D888 | Z18 | Z18 | Z18 |
| 15985~16872 | D1~D888 | Z19 | Z19 | Z19 |
| 16873~17760 | D1~D888 | Z20 | Z20 | Z20 |
| 17761~18648 | D1~D888 | Z21 | Z21 | Z21 |
| 18649~19536 | D1~D888 | Z22 | Z22 | Z22 |
| 19537~20424 | D1~D888 | Z23 | Z23 | Z23 |
| 20425~21312 | D1~D888 | Z24 | Z24 | Z24 |
| 21313~22200 | D1~D888 | Z25 | Z25 | Z25 |
| 22201~23088 | D1~D888 | Z26 | Z26 | Z26 |
| 23089~23976 | D1~D888 | Z27 | Z27 | Z27 |
| 23977~24864 | D1~D888 | Z28 | Z28 | Z28 |
| 24865~25752 | D1~D888 | Z29 | Z29 | Z29 |
| 25753~26640 | D1~D888 | Z30 | Z30 | Z30 |
| 26641~27528 | D1~D888 | Z31 | Z31 | Z31 |
| 27529~28416 | D1~D888 | Z32 | Z32 | Z32 |
| 28417~29304 | D1~D888 | Z33 | Z33 | Z33 |
| 29305~30192 | D1~D888 | Z34 | Z34 | Z34 |
| 30193~31080 | D1~D888 | Z35 | Z35 | Z35 |
| 31081~31968 | D1~D888 | Z36 | Z36 | Z36 |
| 31969~32856 | D1~D888 | Z37 | Z37 | Z37 |
| 32857~33744 | D1~D888 | Z38 | Z38 | Z38 |
| 33745~34632 | D1~D888 | Z39 | Z39 | Z39 |
| 34633~35520 | D1~D888 | Z40 | Z40 | Z40 |
| 35521~36408 | D1~D888 | Z41 | Z41 | Z41 |
| 36409~37296 | D1~D888 | Z42 | Z42 | Z42 |
| 37297~38184 | D1~D888 | Z43 | Z43 | Z43 |
| 38185~39072 | D1~D888 | Z44 | Z44 | Z44 |
| 39073~39960 | D1~D888 | Z45 | Z45 | Z45 |
| 39961~40848 | D1~D888 | Z46 | Z46 | Z46 |
| 40849~41736 | D1~D888 | Z47 | Z47 | Z47 |
| 41737~42624 | D1~D888 | Z48 | Z48 | Z48 |
| 42625~43512 | D1~D888 | Z49 | Z49 | Z49 |
| 43513~44400 | D1~D888 | Z50 | Z50 | 250 |
| 44401~45288 | D1~D888 | Z51 | Z51 | Z51 |
| 45289~46176 | D1~D888 | Z52 | Z52 | Z52 |
| 46177~47064 | D1~D888 | Z53 | Z53 | Z53 |
| 47065~47952 | D1~D888 | Z54 | Z54 | Z54 |
| 47953~48840 | D1~D888 | Z55 | Z55 | Z55 |
| 48841~49728 | D1~D888 | Z56 | Z56 | Z56 |
| 49729~50616 | D1~D888 | Z57 | Z57 | Z57 |
| 50617~51504 | D1~D888 | Z58 | Z58 | Z58 |
| 51505~52392 | D1~D888 | Z59 | Z59 | Z59 |
| 52393~53280 | D1~D888 | Z60 | Z60 | Z60 |
| 53281~54168 | D1~D888 | Z61 | Z61 | Z61 |
| 54169~55056 | D1~D888 | Z62 | Z62 | Z62 |
| 55057~55944 | D1~D888 | Z63 | Z63 | Z63 |
| 55945~56832 | D1~D888 | Z64 | Z64 | Z64 |
| 56833~57720 | D1~D888 | Z65 | Z65 | Z65 |
| 57721~58608 | D1~D888 | Z66 | Z66 | Z66 |
| 58609~59496 | D1~D888 | Z67 | Z67 | Z67 |
| 59497~60384 | D1~D888 | Z68 | Z68 | Z68 |
| 60385~61272 | D1~D888 | Z69 | Z69 | Z69 |
| 61273~62160 | D1~D888 | Z70 | Z70 | Z70 |
| 62161~63048 | D1~D888 | Z71 | Z71 | Z71 |
| 63049~63936 | D1~D888 | Z72 | Z72 | Z72 |
| 83937~64824 | D1~D888 | Z73 | Z73 | Z73 |
| 64825~65712 | D1~D888 | Z74 | Z74 | Z74 |
| 65713~66600 | D1~D888 | Z75 | Z75 | Z75 |
| 66601~67488 | D1~D888 | Z76 | Z76 | Z76 |

In Table 3, structures of Compounds 1 to 1975974 are shown by collectively displaying R¹ to R⁴ of a plurality of compounds in each row.

Compounds 1 to 1463424 in Table 3 specify structures in which 1 to 2 of R¹ to R⁴ is any of D1 to D888, and 2 to 3 of R² to R⁴ are any of Z1 to Z76. In each row in Table 2, Z1 to Z76 are first fixed to one while D1 to D888 are changed in order to specify the compounds, and thereafter Z1 to Z76 are fixed to the next one while D1 to D888 are changed in order to specify the compounds. That is, the row of Compounds 1 to 67488 in Table 3 collectively represents Compounds 1 to 67488 specified in Table 2.

In the row of Compounds 67489 to 134088 in Table 3, those in which R² and R³ are Z2, R⁴ is fixed to Z1 and R¹ is D1 to D888 are Compounds 67489 to 68376 in that order, those in which R² and R³ are Z3, R⁴ is fixed to Z1 and R¹ is D1 to D888 are Compounds 68377 to 69264 in that order, those in which R² and R³ are Z4, R⁴ is fixed to Z1 and R¹ is D1 to D888 are Compounds 69265 to 70152 in that order, and those in which R² and R³ are Z76, R⁴ is fixed to Z1 and R¹ is D1 to D888 are Compounds 11425 to 11592 in that order. In the same manner, Compounds 134089 to 1463424 are also specified.

Compounds 1463425 to 1975974 in Table 3 specify structures in which R¹ is D1, one of R² to R⁴ is Z1, the other one of R² to R⁴ is any of D2 to D1140, and the remaining one of R² to R⁴ is any of Z2 to Z76. Here, Z2 to Z76 are first fixed to one while D2 to D1140 are changed in order to specify the compounds, and thereafter Z2 to Z76 are fixed to the next one while D2 to D1140 are changed in order to specify the compounds. In the row of Compounds 1463425 to 1548849, those in which R¹ is D1, R³ is Z1 and R⁴ is fixed to Z2, and R² is D2 to D1140 are Compounds 1463425 to 1464563 in that order, those in which R¹ is D1, R³ is Z1 and R⁴ is fixed to Z3, and R² is D2 to D1140 are Compounds 1464564 to 1465702, those in which R¹ is D1, R³ is Z1 and R⁴ is fixed to Z4, and R² is D2 to D1140 are Compounds 1465703 to 1466841, and those in which R¹ is D1, R³ is Z1 and R⁴ is fixed to Z35, and R² is D2 to D1140 are Compounds 1547711 to 1548849. In the same manner, Compounds 1548850 to 1975974 are also specified.

TABLE 3

| No. | R¹ | R² | R³ | R⁴ | Note |
|---|---|---|---|---|---|
| 1~67488 | D1~D888 | Z1~Z76 | Z1~Z76 | Z1~Z76 | R² = R³ = R⁴ |
| 67489~134088 | D1~D888 | Z2~Z76 | Z2~Z76 | Z1 | R² = R³ |
| 134089~200688 | D1~D888 | Z2~Z76 | Z1 | Z2~Z76 | R² = R⁴ |
| 200689~267288 | D1~D888 | Z1 | Z2~Z76 | Z2~Z76 | R³ = R⁴ |
| 267289~333888 | D1~D888 | Z1 | Z1 | Z2~Z76 | R² = R³ |
| 333889~400488 | D1~D888 | Z1 | Z2~Z76 | Z1 | R² = R⁴ |
| 400489~467088 | D1~D888 | Z2~Z76 | Z1 | Z1 | R³ = R⁴ |
| 467089~532800 | D1~D888 | Z1 | Z2 | Z3~Z76 | |
| 532801~598512 | D1~D888 | Z1 | Z3~Z76 | Z2 | |
| 598513~664224 | D1~D888 | Z3~Z76 | Z1 | Z2 | |
| 664225~729936 | D1~D888 | Z2 | Z1 | Z3~Z76 | |
| 729937~795648 | D1~D888 | Z2 | Z3~Z76 | Z1 | |
| 795649~861360 | D1~D888 | Z3~Z76 | Z2 | Z1 | |
| 861361~928848 | D1~D888 | D1~D888 | Z1~Z76 | Z1~276 | R¹ = R², R³ = R⁴ |
| 928849~996336 | D1~D888 | Z1~Z76 | D1~D888 | Z1~276 | R¹ = R³, R² = R⁴ |
| 996337~1063824 | D1~D888 | Z1~Z76 | Z1~Z76 | D1~D888 | R¹ = R⁴, R² = R³ |
| 1063825~1130424 | D1~D888 | D1~D888 | Z1 | Z2~Z76 | R¹ = R² |
| 1130425~1197024 | D1~D888 | Z1 | D1~D888 | Z2~Z76 | R¹ = R³ |
| 1197025~1263624 | D1~D888 | Z1 | Z2~Z76 | D1~D888 | R¹ = R⁴ |
| 1263625~1330224 | D1~D888 | D1~D888 | Z2~Z76 | Z1 | R¹ = R² |
| 1330225~1396824 | D1~D888 | Z2~Z76 | D1~D888 | Z1 | R¹ = R³ |
| 1396825~1463424 | D1~D888 | Z2~Z76 | Z1 | D1~D888 | R¹ = R⁴ |
| 1463425~1548849 | D1 | D2~D1140 | Z1 | Z2~Z76 | |
| 1548850~1634274 | D1 | Z1 | D2~D1140 | Z2~Z76 | |
| 1634275~1719699 | D1 | Z1 | Z2~Z76 | D2~D1140 | |
| 1719700~1805124 | D1 | D2~D1140 | Z2~Z76 | Z1 | |
| 1805125~1890549 | D1 | Z2~Z76 | D2~D1140 | Z1 | |
| 1890550~1975974 | D1 | Z2~Z76 | Z1 | D2~D1140 | |

The compounds specified by the above numbers are all individually disclosed. In addition, among the specific examples of the compounds, in the case where a rotamer is present, a mixture of rotamers and each separated rotamer are also disclosed in the description herein.

In one aspect of the present invention, compounds are selected from Compounds 1 to 1975974.

In one aspect of the present invention, compounds are selected from Compounds 1 to 861360. In one aspect of the present invention, compounds are selected from Compounds 861361 to 1975974.

In one aspect of the present invention, compounds are selected from Compounds 1 to 67488. In one aspect of the present invention, compounds are selected from Compounds 67489 to 467088. In one aspect of the present invention, compounds are selected from Compounds 467089 to 861360. In one aspect of the present invention, compounds are selected from Compounds 861361 to 1063824. In one aspect of the present invention, compounds are selected from Compounds 1608325 to 1463424. In one aspect of the present invention, compounds are selected from Compounds 1463425 to 1975974.

In one aspect of the present invention, compounds are selected from Compounds 67489 to 467088, and 1063825 to 1463424. In one aspect of the present invention, compounds are selected from Compounds 467089 to 861360, and 1463425 to 1975974.

In one aspect of the present invention, compounds are selected from Compounds 861361 to 928848, 1063825 to 1130424, 1263625 to 1330224, 1463425 to 1548849, and 1719700 to 1805124. In one aspect of the present invention, compounds are selected from Compounds 928849 to 996336, 1130425 to 1197024, 1330225 to 1396824, 1548850 to 1634274, and 1805125 to 1890549. In one aspect of the present invention, compounds are selected from Compounds 996337 to 1063824, 1197025 to 1263624, 1396825 to 1463424, 1634275 to 1719699, 1890550 to 1975974.

In the above, specific examples of compounds where all the substituted or unsubstituted aryl groups are an unsubstituted phenyl group (Ar1), or a phenyl group substituted with a deuterium atom (Ar21), as included in D1 to D888, are specified as Compounds 1 to 1975974. In Table 4, compounds obtained by changing the unsubstituted phenyl group (Ar1) or the phenyl group substituted with a deuterium atom (Ar21) of the substituted or unsubstituted aryl group (Ar¹, Ar²) existing in D1 to D175308 that Compounds 1 to 1975974 have, to those as in Table 4 are sequentially shown in the tabular form. In Table 4, Compounds 1 to 1975974 are also shown for clarifying the correspondence relationship. For example, Compound 1 (1) is a compound having a structure in which the unsubstituted phenyl group (Ar1) existing in D1 of Compound 1 is substituted with a 2-naphthyl group (Ar2). Compound 2 (1) is a compound having a structure in which the unsubstituted phenyl group (Ar1) existing in D2 of Compound 2 is substituted with a 2-naphthyl group (Ar2). Compound 1975974 (1) is a compound having a structure in which the unsubstituted phenyl group (Ar1) existing in D1 of Compound 1975974 is substituted with a 2-naphthyl group (Ar2). Compounds 1 (2) to 1975974 (2) and the subsequent compounds are specified in the same manner. In Table 4, Ar¹ and Ar² are the same in the compounds.

TABLE 4

| No. | Ar¹, Ar² |
|---|---|
| 1~1975974 | Ar1 or Ar21 |
| 1(1)~1975974(1) | Ar2 |
| 1(2)~1975974(2) | Ar3 |
| 1(3)~1975974(3) | Ar4 |
| 1(4)~1975974(4) | Ar5 |
| 1(5)~1975974(5) | Ar6 |
| 1(6)~1975974(6) | Ar7 |
| 1(7)~1975974(7) | Ar8 |
| 1(8)~1975974(8) | Ar9 |
| 1(9)~1975974(9) | Ar10 |
| 1(10)~1975974(10) | Ar11 |
| 1(11)~1975974(11) | Ar12 |
| 1(12)~1975974(12) | Ar13 |
| 1(13)~1975974(13) | Ar14 |
| 1(14)~1975974(14) | Ar15 |
| 1(15)~1975974(15) | Ar16 |
| 1(16)~1975974(16) | Ar17 |
| 1(17)~1975974(17) | Ar18 |
| 1(18)~1975974(18) | Ar19 |
| 1(19)~1975974(19) | Ar20 |
| 1(20)~1975974(20) | Ar22 |
| 1(21)~1975974(21) | Ar23 |
| 1(22)~1975974(22) | Ar24 |
| 1(23)~1975974(23) | Ar25 |
| 1(24)~1975974(24) | Ar26 |
| 1(25)~1975974(25) | Ar27 |
| 1(26)~1975974(26) | Ar28 |
| 1(27)~1975974(27) | Ar29 |
| 1(28)~1975974(28) | Ar30 |
| 1(29)~1975974(29) | Ar31 |
| 1(30)~1975974(30) | Ar32 |
| 1(31)~1975974(31) | Ar33 |
| 1(32)~1975974(32) | Ar34 |
| 1(33)~1975974(33) | Ar35 |
| 1(34)~1975974(34) | Ar36 |
| 1(35)~1975974(35) | Ar37 |
| 1(36)~1975974(36) | Ar38 |
| 1(37)~1975974(37) | Ar39 |
| 1(38)~1975974(38) | Ar40 |
| 1(39)~1975974(39) | Ar41 |
| 1(40)~1975974(40) | Ar42 |
| 1(41)~1975974(41) | Ar43 |
| 1(42)~1975974(42) | Ar44 |
| 1(43)~1975974(43) | Ar45 |
| 1(44)~1975974(44) | Ar46 |
| 1(45)~1975974(45) | Ar47 |
| 1(46)~1975974(46) | Ar48 |
| 1(47)~1975974(47) | Ar49 |
| 1(48)~1975974(48) | Ar50 |
| 1(49)~1975974(49) | Ar51 |
| 1(50)~1975974(50) | Ar52 |
| 1(51)~1975974(51) | Ar53 |
| 1(52)~1975974(52) | Ar54 |
| 1(53)~1975974(53) | Ar55 |
| 1(54)~1975974(54) | Ar56 |
| 1(55)~1975974(55) | Ar57 |

In one preferred aspect of the present invention, the compound represented by the general formula (1) is selected from the following group of compounds.

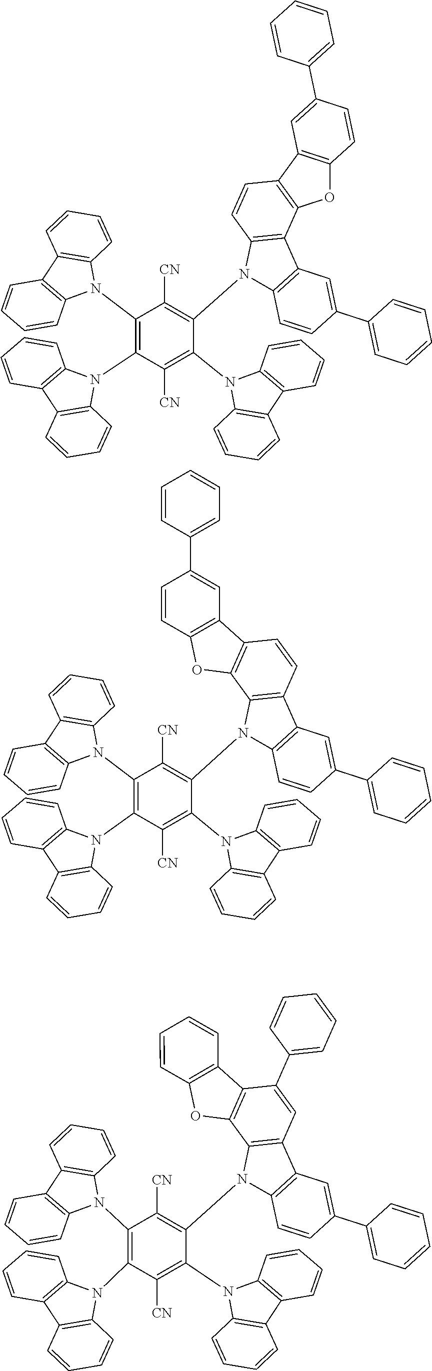

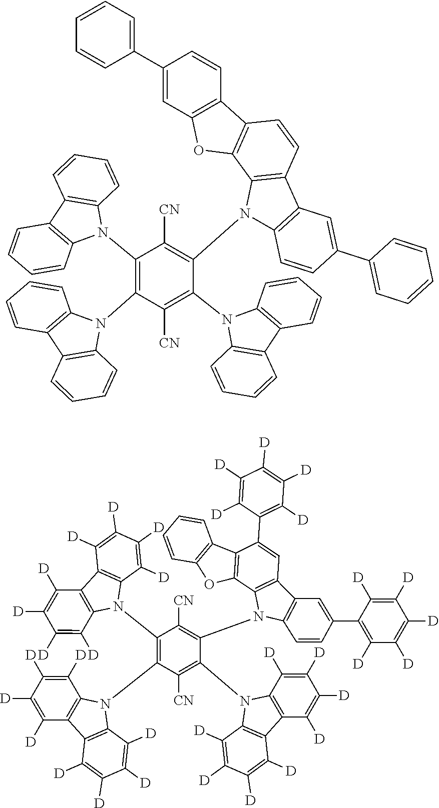

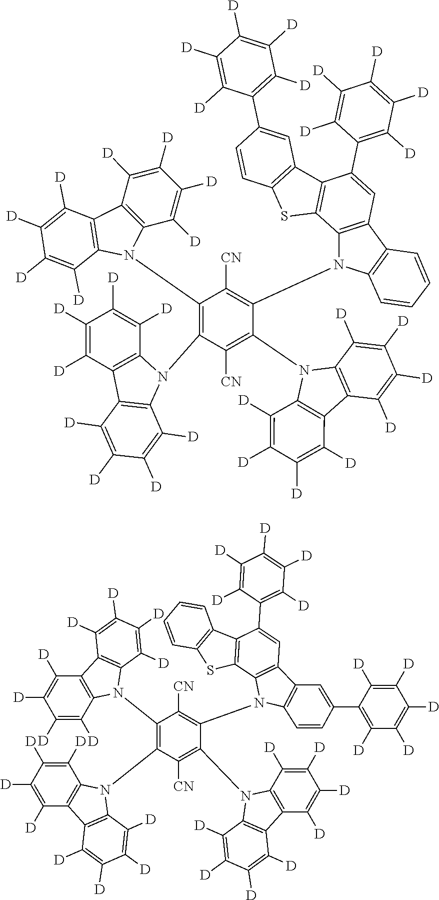

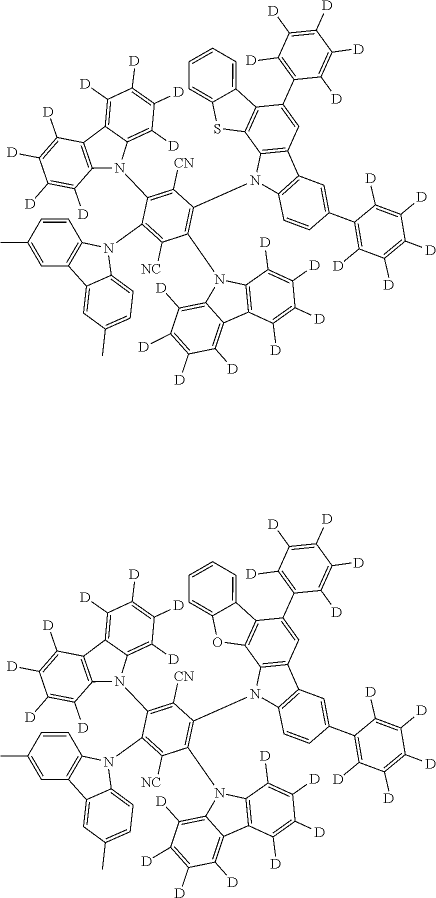

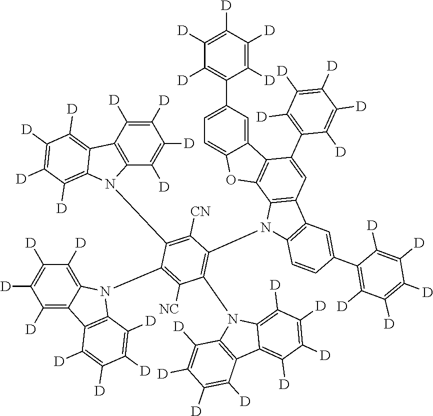

The molecular weight of the compound represented by the general formula (1) is preferably 1500 or less, and more preferably 1200 or less, for example, in the case where an organic layer containing the compound represented by the general formula (1) is intended to be film-formed by a vapor deposition method, and used as a film. The lower limit of the molecular weight is the molecular weight of the minimum compound represented by the general formula (1).

The compound represented by the general formula (1) can be formed into a film by a coating method regardless of the molecular weight. When the coating method is used, even a compound having a relatively large molecular weight can be formed into a film. The compound represented by the general formula (1) has an advantage of being easily dissolved in an organic solvent. For this reason, the compound represented by the general formula (1) is easily applicable to a coating method and is easily purified to increase its purity.

It is also conceivable to use a compound containing a plurality of structures represented by the general formula (1) in a molecule as a light emitting material by applying the present invention.

For example, it is conceivable that a polymer obtained by allowing a polymerizable group to be present in the structure represented by the general formula (1) in advance and polymerizing the polymerizable group is used as the light emitting material. For example, it is conceivable that a polymer having a repeating unit is obtained by preparing a monomer containing a polymerizable functional group at any site of the general formula (1) and polymerizing the monomer alone or copolymerizing the monomer with another monomer, and the polymer is used as the light emitting material. Alternatively, it is also conceivable to obtain a dimer or a trimer by coupling compounds having a structure represented by the general formula (1) to each other and to use the dimer or the trimer as a light emitting material.

Examples of the polymer having a repeating unit containing a structure represented by the general formula (1) include polymers containing a structure represented by any one of the following two general formulae.

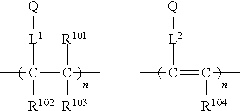

In the above general formulae, Q represents a group containing the structure represented by the general formula (1), and L¹ and L² represent a linking group. The linking group preferably has 0 to 20 carbon atoms, more preferably 1 to 15 carbon atoms, and further preferably 2 to 10 carbon atoms. The linking group preferably has a structure represented by —X¹¹-L¹¹-. Here, X¹¹ represents an oxygen atom or a sulfur atom, and is preferably an oxygen atom. L¹¹ represents a linking group, and is preferably a substituted or unsubstituted alkylene group or a substituted or unsubstituted arylene group, and more preferably a substituted or unsubstituted alkylene group having 1 to 10 carbon atoms or a substituted or unsubstituted phenylene group.

In the above general formulae, R¹⁰¹, R¹⁰², R¹⁰³ and R¹⁰⁴ each independently represent a substituent. It is preferably a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and further preferably an unsubstituted alkyl group having 1 to 3 carbon atoms or an unsubstituted alkoxy group having 1 to 3 carbon atoms.

The linking group represented by L¹ and L² can bond to any site of the general formula (1) constituting Q. Two or more linking groups can be linked to one Q to form a cross-linked structure or a network structure.

Specific structural examples of the repeating unit include structures represented by the following formulae.

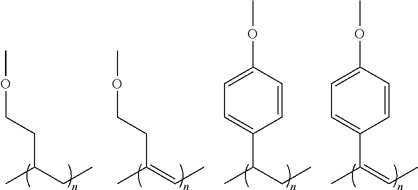

The polymer having a repeating unit including these formulae can be synthesized by introducing a hydroxy group into any site of the general formula (1), reacting the following compound using the hydroxy group as a linker to introduce a polymerizable group, and polymerizing the polymerizable group.

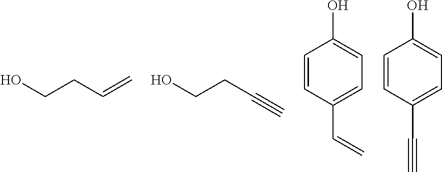

The polymer having a structure represented by the general formula (1) in the molecule can be a polymer having only a repeating unit that has the structure represented by the general formula (1), or can be a polymer containing a repeating unit that has any other structure. The repeating unit having the structure represented by the general formula (1) to be contained in the polymer can be a single kind or two or more kinds. The repeating unit not having the structure represented by the general formula (1) includes those derived from monomers used in general copolymerization. For example, it includes repeating units derived from monomers having an ethylenically unsaturated bond, such as ethylene or styrene.

In some embodiments, the compound represented by the general formula (1) is a light emitting material.

In some embodiments, the compound represented by the general formula (1) is a compound capable of emitting delayed fluorescence.

In some embodiments of the present disclosure, the compound represented by the general formula (1) is, when excited thermally or by an electronic means, able to emit light in a UV region, emit light of blue, green, yellow, orange, or red in a visible spectral region (e.g., about 420 nm to about 500 nm, about 500 nm to about 600 nm, or about 600 nm to about 700 nm) or emit light in a near IR region.

In some embodiments of the present disclosure, the compound represented by the general formula (1) is, when excited thermally or by an electronic means, able to emit light in an orange or yellow region of a visible spectrum (e.g., about 570 nm to about 620 nm, for example, 570 to 590 nm).

In some embodiments of the present disclosure, an organic semiconductor device using the compound represented by the general formula (1) can be produced. The organic semiconductor device referred to herein can be an organic optical device in which light is interposed or an organic device in which light is not interposed. The organic optical device can be an organic light emitting device in which the device emits light, an organic light receiving device in which the device receives light, or a device in which energy transfer by light occurs in the device. In some embodiments of the present disclosure, an organic optical device such as an organic electroluminescent device or a solid-state imaging device (for example, a CMOS image sensor) can be produced by using the compound represented by the general formula (1). In some embodiments of the present disclosure, a CMOS (complementary metal-oxide semiconductor) or the like using the compound represented by the general formula (1) can be produced.

Electronic characteristics of small-molecule chemical substance libraries can be calculated by known ab initio quantum chemistry calculation. For example, according to time-dependent density functional theory calculation using 6-31G* as a basis, and a functional group known as Becke's three parameters, Lee-Yang-Parr hybrid functionals, the Hartree-Fock equation (TD-DFT/B3LYP/6-31G*) is analyzed and molecular fractions (parts) having HOMO not lower than a specific threshold value and LUMO not higher than a specific threshold value can be screened.

With that, for example, in the presence of a HOMO energy (for example, ionizing potential) of −6.5 eV or more, a donor part (“D”) can be selected. On the other hand, for example, in the presence of a LUMO energy (for example, electron affinity) of −0.5 eV or less, an acceptor part (“A”) can be selected. A bridge part (“B”) is a strong conjugated system, for example, capable of strictly limiting the acceptor part and the donor part in a specific three-dimensional configuration, and therefore prevents the donor part and the acceptor part from overlapping in the x-conjugated system.

In some embodiments, a compound library is screened using at least one of the following characteristics.
1. Light emission around a specific wavelength
2. A triplet state over a calculated specific energy level
3. ΔE_{ST} value lower than a specific value
4. Quantum yield more than a specific value
5. HOMO level
6. LUMO level

In some embodiments, the difference (ΔE_{ST}) between the lowest singlet excited state and the lowest triplet excited state at 77 K is less than about 0.5 eV, less than about 0.4 eV, less than about 0.3 eV, less than about 0.2 eV, or less than about 0.1 eV. In some embodiments, ΔE_{ST} value is less than about 0.09 eV, less than about 0.08 eV, less than about 0.07 eV, less than about 0.06 eV, less than about 0.05 eV, less than about 0.04 eV, less than about 0.03 eV, less than about 0.02 eV, or less than about 0.01 eV.

In some embodiments, the compound represented by the general formula (1) shows a quantum yield of more than 25%, for example, about 30%, about 35%, about 40%, about 45%, about 50%, about 55%, about 60%, about 65%, about 70%, about 75%, about 80%, about 85%, about 90%, about 95% or more.

### [Method for Synthesizing Compound Represented by General Formula (1)]

The compound represented by the general formula (1) includes a novel compound.

The compound represented by the general formula (1) can be synthesized by combining known reactions. For example, a compound of the general formula (1) where R¹ is a halogen atom is first synthesized, and then reacted with a ring-fused carbazole in which the ring skeleton constituting carbon atoms are substituted with at least two substituted or unsubstituted aryl groups to give the compound of the general formula (1). For details of the reaction conditions, Synthesis Examples described later can be referred to.

### [Structure Using Compound Represented by General Formula (1)]

In some embodiments, the compound represented by the general formula (1) is used along with one or more materials (e.g., small molecules, polymers, metals, metal complexes), by combining them, or by dispersing the compound, or by covalent-bonding with the compound, or by coating with the compound, or by carrying the compound, or by associating with the compound, and solid films or layers are formed. For example, by combining the compound represented by the general formula (1) with an electroactive material, a film can be formed. In some cases, the compound represented by the general formula (1) can be combined with a hole transporting polymer. In some cases, the compound represented by the general formula (1) can be combined with an electron transporting polymer. In some cases, the compound represented by the general formula (1) can be combined with a hole transporting polymer and an electron transporting polymer. In some cases, the compound represented by the general formula (1) can be combined with a copolymer having both a hole transporting moiety and an electron transporting moiety. In the embodiments mentioned above, the electrons and/or the holes formed in a solid film or layer can be interacted with the compound represented by the general formula (1).

### [Film Formation]

In some embodiments, a film containing the compound represented by the general formula (1) can be formed in a wet process. In a wet process, a solution prepared by dissolving a composition containing the compound of the present invention is applied onto a surface, and then the solvent is removed to form a film. The wet process includes a spin coating method, a slit coating method, an inkjet method (a spraying method), a gravure printing method, an offset printing method and flexographic printing method, which, however are not limitative. In the wet process, an appropriate organic solvent capable of dissolving a composition containing the compound of the present invention is selected and used. In some embodiments, a substituent (e.g., an alkyl group) capable of increasing the solubility in an organic solvent can be introduced into the compound contained in the composition.

In some embodiments, a film containing the compound of the present invention can be formed in a dry process. In some embodiments, a vacuum deposition method is employable as a dry process, which, however, is not limitative. In the case where a vacuum deposition method is employed, compounds to constitute a film can be co-deposited from individual vapor deposition sources, or can be co-deposited from a single vapor deposition source formed by mixing the compounds. In the case where a single vapor deposition source is used, a mixed powder prepared by mixing compound powders can be used, or a compression molded body prepared by compression-molding the mixed powder can be used, or a mixture prepared by heating and melting the compounds and cooling the resulting melt can be used. In some embodiments, by co-deposition under the condition where the vapor deposition rate (weight reduction rate) of the plural compounds contained in a single vapor deposition source is the same or is nearly the same, a film having a compositional ratio corresponding to the compositional ratio of the plural compounds contained in the vapor deposition source can be formed. When plural compounds are mixed in the same compositional ratio as the compositional ratio of the film to be formed to prepare a vapor deposition source, a film having a desired compositional ratio can be formed in a simplified manner. In some embodiments, the temperature at which the compounds to be co-deposited have the same weight reduction ratio is specifically defined, and the temperature can be employed as the temperature of co-deposition.

### [Use Examples of Compound Represented by General Formula (1)]

The compound represented by the general formula (1) is useful as a material for an organic light emitting device. In particular, the compound is preferably used for an organic light emitting diode or the like.

### Organic Light Emitting Diode:

One embodiment of the present invention relates to use of the compound represented by the general formula (1) of the present invention as a light emitting material for organic light emitting devices. In some embodiments, the compound represented by the general formula (1) of the present invention can be effectively used as a light emitting material in a light emitting layer in an organic light emitting device. In some embodiments, the compound represented by the general formula (1) includes a delayed fluorescent material that emits delayed fluorescence. In some embodiments, the present invention provides a delayed fluorescent material having a structure represented by the general formula (1). In some embodiments, the present invention relates to use of the compound represented by the general formula (1) as a delayed fluorescent material. In some embodiments, the compound represented by the general formula (1) of the present invention can be used as a host material, and can be used along with one or more light emitting materials, and the light emitting material can be a fluorescent material, a phosphorescent material or a TADF. In some embodiments, the compound represented by the general formula (1) can be used as a hole transporting material. In some embodiments, the compound represented by the general formula (1) can be used as an electron transporting material. In some embodiments, the present invention relates to a method of generating delayed fluorescence from the compound represented by the general formula (1). In some embodiments, the organic light emitting device containing the compound as a light emitting material emits delayed fluorescence and shows a high light emission efficiency.

In some embodiments, the light emitting layer contains the compound represented by the general formula (1), and the compound represented by the general formula (1) is aligned in parallel to the substrate. In some embodiments, the substrate is a film-forming surface. In some embodiment, the alignment of the compound represented by the general formula (1) relative to the film-forming surface can have some influence on the propagation direction of light emitted by the aligned compounds, or can determine the direction. In some embodiments, by aligning the propagation direction of light emitted by the compound represented by the general formula (1), the light extraction efficiency from the light emitting layer can be improved.

One aspect of the present invention relates to an organic light emitting device. In some embodiments, the organic light emitting device includes a light emitting layer. In some embodiments, the light emitting layer contains, as a light emitting material, the compound represented by the general formula (1). In some embodiments, the organic light emitting device is an organic photoluminescent device (organic PL device). In some embodiments, the organic light emitting device is an organic electroluminescent device (organic EL device). In some embodiments, the compound represented by the general formula (1) assists light irradiation from the other light emitting materials contained in the light emitting layer (as a so-called assist dopant). In some embodiments, the compound represented by the general formula (1) contained in the light emitting layer is in a lowest excited singlet energy level, and is contained between the lowest excited single energy level of the host material contained in the light emitting layer and the lowest excited singlet energy level of the other light emitting materials contained in the light emitting layer.

In some embodiments, the organic photoluminescent device comprises at least one light emitting layer. In some embodiments, the organic electroluminescent device includes at least an anode, a cathode, and an organic layer between the anode and the cathode. In some embodiments, the organic layer includes at least a light emitting layer. In some embodiments, the organic layer includes only a light emitting layer. In some embodiments, the organic layer includes one or more organic layers in addition to the light emitting layer. Examples of the organic layer include a hole transporting layer, a hole injection layer, an electron barrier layer, a hole barrier layer, an electron injection layer, an electron transporting layer and an exciton barrier layer. In some embodiments, the hole transporting layer can be a hole injection and transporting layer having a hole injection function, and the electron transporting layer can be an electron injection and transporting layer having an electron injection function.

### Light Emitting Layer:

In some embodiments, the light emitting layer is a layer where holes and electrons injected from the anode and the cathode, respectively, are recombined to form excitons. In some embodiments, the layer emits light.

In some embodiments, only a light emitting material is used as the light emitting layer. In some embodiments, the light emitting layer contains a light emitting material and a host material. In some embodiments, the light emitting material is one or more compounds represented by the general formula (1). In some embodiments, for improving luminous radiation efficiency of an organic electroluminescent device and an organic photoluminescent device, the singlet exciton and the triplet exciton generated in a light emitting material are confined inside the light emitting material. In some embodiments, a host material is used in the light emitting layer in addition to a light emitting material. In some embodiments, the host material is an organic compound. In some embodiments, the organic compound has an excited singlet energy and an excited triplet energy, and at least one of them is higher than those in the light emitting material of the present invention. In some embodiments, the singlet exciton and the triplet exciton generated in the light emitting material of the present invention are confined in the molecules of the light emitting material of the present invention. In some embodiments, the singlet and triplet excitons are fully confined for improving luminous radiation efficiency. In some embodiments, although high luminous radiation efficiency is still attained, singlet excitons and triplet excitons are not fully confined, that is, a host material capable of attaining high luminous radiation efficiency can be used in the present invention with no specific limitation. In some embodiments, in the light emitting material in the light emitting layer of the device of the present invention, luminous radiation occurs. In some embodiments, radiated light includes both fluorescence and delayed fluorescence. In some embodiments, radiated light includes radiated light from a host material. In some embodiments, radiated light is composed of radiated light from a host material. In some embodiments, radiated light includes radiated light from the compound represented by the general formula (1) and radiated light from a host material. In some embodiment, a TADF molecule and a host material are used. In some embodiments, TADF is an assist dopant and has a lower excited singlet energy than the host material in the light emitting layer and a higher excited singlet energy than the light emitting material in the light emitting layer.

In the case where the compound represented by the general formula (1) is used as an assist dopant, various compounds can be employed as a light emitting material (preferably a fluorescent material). As such light emitting materials, employable are an anthracene derivative, a tetracene derivative, a naphthacene derivative, a pyrene derivative, a perylene derivative, a chrysene derivative, a rubrene derivative, a coumarin derivative, a pyran derivative, a stilbene derivative, a fluorene derivative, an anthryl derivative, a pyrromethene derivative, a terphenyl derivative, a terphenylene derivative, a fluoranthene derivative, an amine derivative, a quinacridone derivative, an oxadiazole derivative, a malononitrile derivative, a pyran derivative, a carbazole derivative, a julolidine derivative, a thiazole derivative, and a derivative having a metal (Al, Zn). These exemplified skeletons can have a substituent, or may not have a substituent. These exemplified skeletons can be combined.

Examples of the light emitting material that can be used in combination with an assist dopant having a structure represented by the general formula (1) include compounds represented by the following general formula (4).

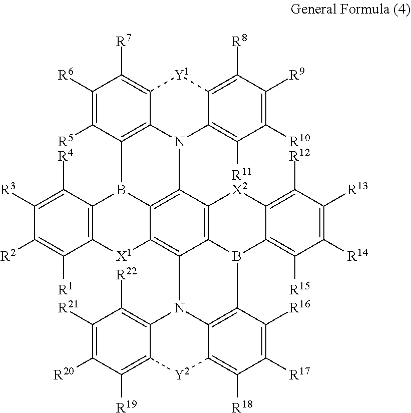

In the general formula (4), X¹ and X² each independently represent O or S. Y¹ and Y² each independently represent a single bond, O, S or C(R^{a})(R^{b}). R¹ to R²², R^{a}, and R^{b} each independently represent a hydrogen atom, a deuterium atom or a substituent, but at least one of R¹ to R²² is a substituent. R¹ and R², R² and R³, R³ and R⁴, R⁵ and R⁶, R⁶ and R⁷, R⁷ and Y¹, Y¹ and R⁸, R⁸ and R⁹, R⁹ and R¹⁰, R¹⁰ and R¹¹, R¹² and R¹³, R¹³ and R¹⁴, R¹⁴ and R¹⁵, R¹⁶ and R¹⁷, R¹⁷ and R¹⁸, R¹⁸ and Y², Y² and R¹⁹, R¹⁹ and R²⁰, R²⁰ and R²¹, and R²¹ and R²² each can bond to each other to form a cyclic structure. In one preferred aspect, these do not bond to each other to form a cyclic structure. R²¹ and R¹, R⁴ and R⁵, R¹⁰ and R¹², and R¹⁵ and R¹⁶ each do not bond to each other to form a cyclic structure. In the general formula (1), C—R¹, C—R², C—R³, C—R⁴, C—R³, C—R⁶, C—R⁷, C—R⁸, C—R⁹, C—R¹⁰, C—R¹¹, C—R¹², C—R¹³, C—R¹⁴, C—R¹⁵, C—R¹⁶, C—R¹⁷, C—R¹⁸, C—R¹⁹, C—R²⁰, C—R²¹, and C—R²² can be substituted with N, but are preferably not substituted with N.

Preferably, R¹ to R²² each are a group selected from the group consisting of a hydrogen atom, a deuterium atom, an alkyl group and an aryl group or a group formed by combining at least two thereof, in which a part or all of the hydrogen atoms existing in the group can be substituted with deuterium atoms, or each are a substituted or unsubstituted diarylamino group, in which the two aryl groups constituting the diarylamino group can be linked to each other via a linking group. Preferably, at least one of R¹ to R²² is an aryl group optionally substituted with at least one selected from the group consisting of a deuterium atom, an alkyl group and an aryl group. Preferably, among R¹ to R²², only 1 to 6 selected from the group consisting of R², R³, R⁶, R⁹, R¹³, R¹⁴, R¹⁷, and R²⁰ are substituents. Preferably, the total carbon number of R¹ to R²² is 10 to 60, and also preferably, the total number of the benzene rings of R¹ to R²² is 2 to 6. In one preferred aspect, Y¹ and Y² each are a single bond. In one preferred aspect, X¹ and X² are O. In one preferred aspect, the compound has a point-symmetrical structure.

The compound represented by the general formula (4) preferably has, for example, any of the following skeleton structures. In one preferred aspect, the compound has any skeleton of the following Group 1. In one preferred aspect, the compound has any skeleton of the following Group 2. In one preferred aspect, the compound has any skeleton of the following Group 3. In one preferred aspect, the compound has any skeleton of the following Group 4. In one preferred aspect, the compound has any skeleton of the following Group 5. In the following skeleton structures, X represents O or S, Y represents O, S or C(R^{a})(R^{b}), preferably O or S, and R^{a} and R^{b} are as defined in the general formula (4). At least one hydrogen atom in the following skeleton can be substituted with a substituent containing a deuterium atom. The substituent can be selected from Substituent Group A, can be selected from Substituent Group B, can be selected from Substituent Group C, can be selected from Substituent Group D, or can be selected from Substituent Group E.

[chemical structure]

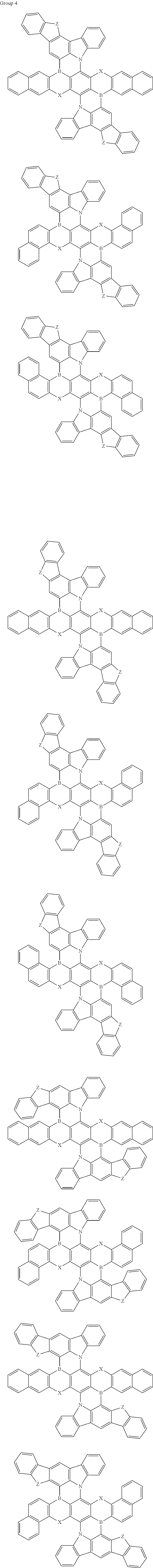

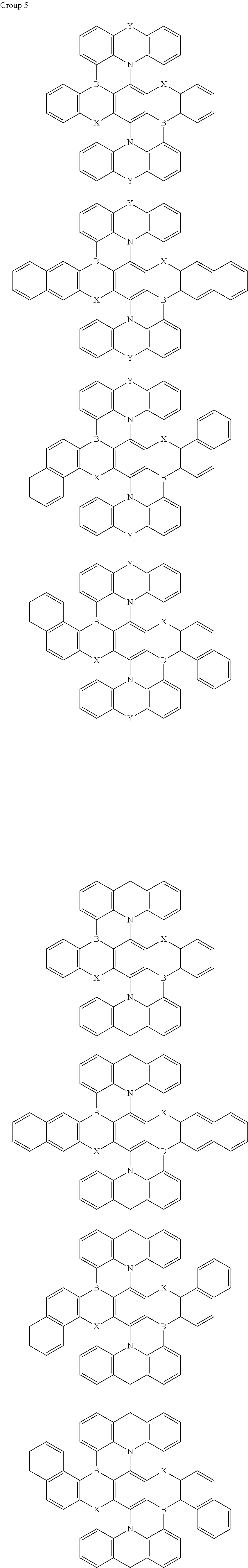

Specific examples of the compound represented by the general formula (4) are shown below, but the compound represented by the general formula (4) that can be used in the present invention should not be construed as being limited by these specific examples.

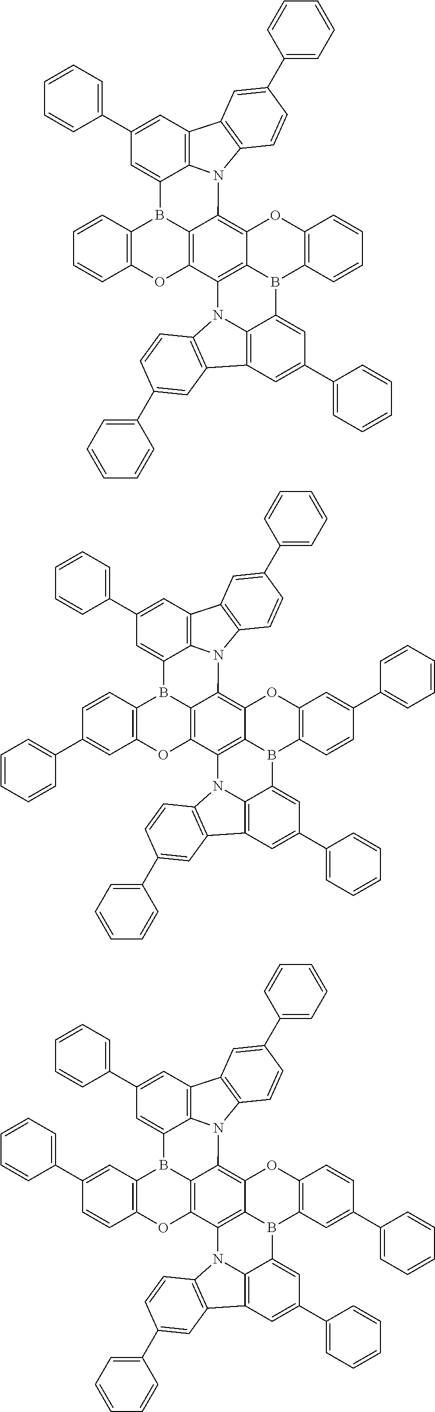

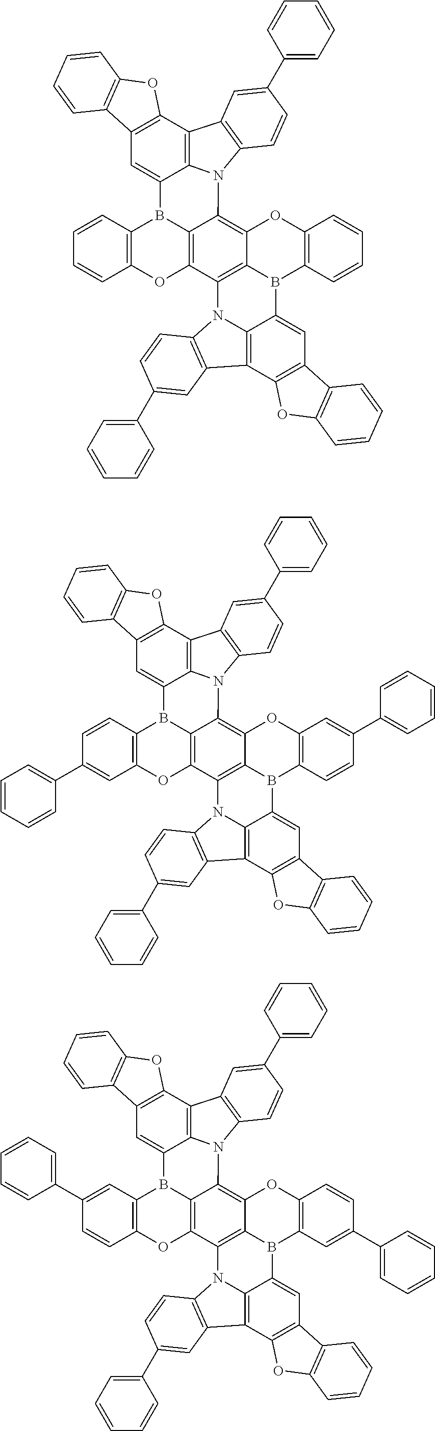

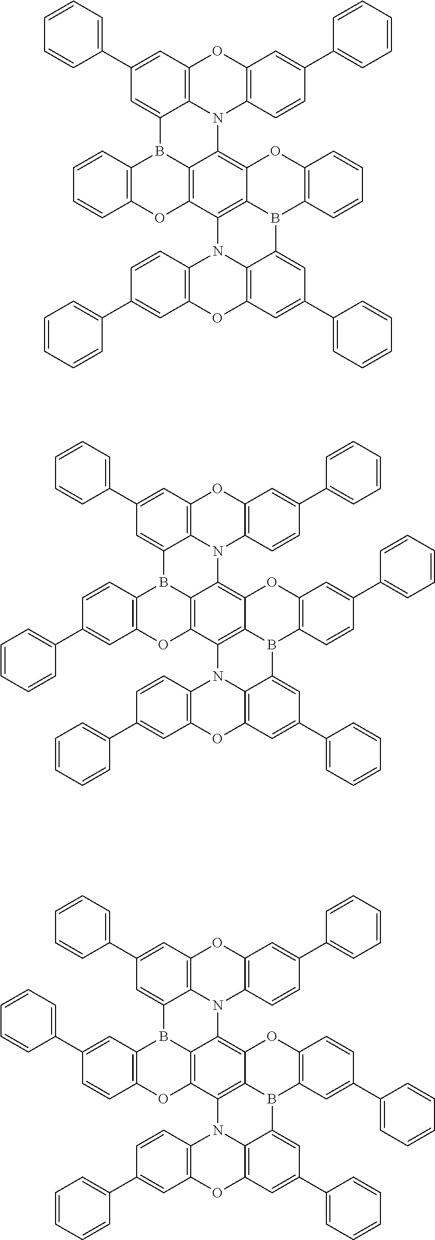

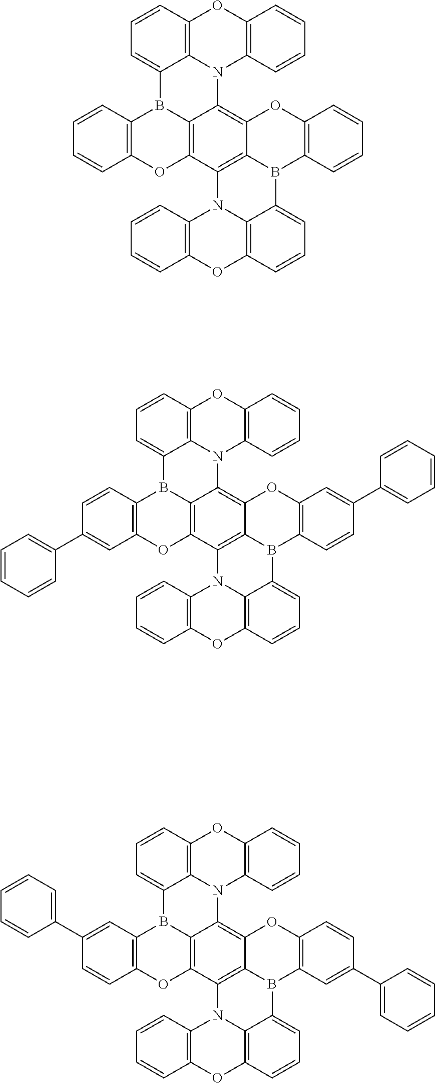

Examples of the light emitting material that can be used in combination with an assist dopant having a structure represented by the general formula (1) include pyrromethene-boron complex compounds. Examples are pyrromethene-boron complex compounds represented by the following general formula (5).

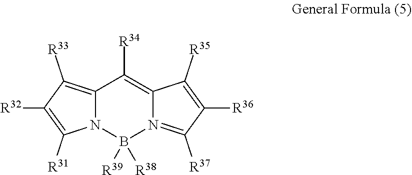

In the general formula (5), R³¹ to R³⁷ each independently represent a hydrogen atom or a substituent, and the substituent includes a deuterium atom, and R³¹ and R³², R³² and R³³, R³³ and R³⁴, R³⁴ and R³⁵, R³⁵ and R³⁶, and R³⁶ and R³⁷ each can bond to each other to form a cyclic structure. R³⁸ and R³⁹ each independently represent an atom or a group selected from the group consisting of a halogen atom, an alkyl group, an aryl group, an alkoxy group and an aryloxy group, or a group formed by combining at least two such atoms and groups.

Specific examples of the compound represented by the general formula (5) include compounds described in WO2012/015177, paragraphs 0196 to 0255.

Examples of the light emitting material that can be used in combination with an assist dopant having a structure represented by the general formula (1) include compounds described in JP-A 2022-027733, paragraph 0050, and compounds described in WO2015/022974, paragraphs 0220 to 0239.

Compounds represented by the following general formula (E1) are further preferred light emitting materials.

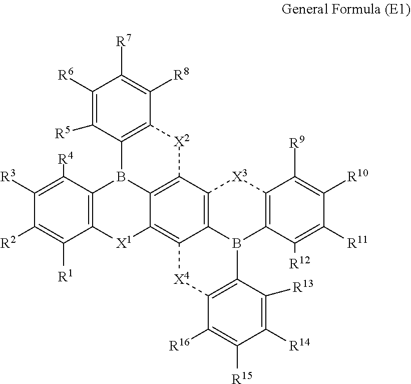

In the general formula (E1), R¹ and R³ to R¹⁶ each independently represent a hydrogen atom, a deuterium atom, or a substituent. R² represents an acceptor group, or R¹ and R² bond to each other to form an acceptor group, or R² and R³ bond to each other to form an acceptor group. R³ and R⁴, R⁴ and R⁵, R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each can bond to each other to form a cyclic structure. X¹ represents O or NR, and R represents a substituent. Of X² to X⁴, at least one of X³ and X⁴ is O or NR, and the remainder may be O or NR, or unlinked. When not linked, both ends each independently represent a hydrogen atom, a deuterium atom or a substituent. In the general formula (1), C—R¹, C—R³, C—R⁴, C—R⁵, C—R⁶, C—R⁷, C—R⁸, C—R⁹, C—R¹⁰, C—R¹¹, C—R¹², C—R¹³, C—R¹⁴, C—R¹⁵, and C—R¹⁶ can be substituted with N.

Compounds represented by the following general formula (E2) are further preferred light emitting materials.

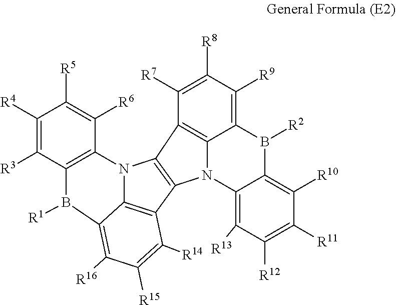

In the general formula (E2), R¹ and R² each independently represent a substituted or unsubstituted alkyl group, a substituted or unsubstituted aryl group, or a substituted or unsubstituted heteroaryl group, and R³ to R¹⁶ each independently represent a hydrogen atom, a deuterium atom or a substituent. R¹ and R³, R³ and R⁴, R⁴ and R⁵, R⁵ and R⁶, R⁶ and R⁷, R⁷ and R⁸, R⁸ and R⁹, R⁹ and R², R² and R¹⁰, R¹⁰ and R¹¹, R₁₁ and R¹², R¹² and R¹³, R¹³ and R¹⁴, R¹⁴ and R¹⁵, R¹⁵ and R¹⁶, and R¹⁶ and R¹ each can bond to each other to form a cyclic structure. In the general formula (1), C—R³, C—R⁴, C—R⁵, C—R⁶, C—R⁷, C—R⁸, C—R⁹, C—R¹⁰, C—R¹¹, C—R¹², C—R¹³, C—R¹⁴, C—R¹⁵, and C—R¹⁶ can be substituted with N.

Compounds represented by the following general formula (E3) are further preferred light emitting materials.

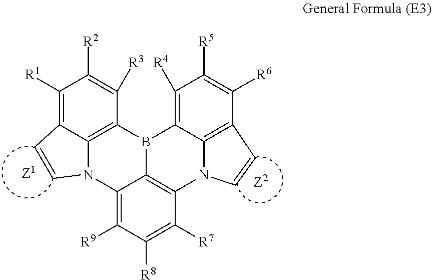

In the general formula (E3), Z1 and Z2 each independently represent a substituted or unsubstituted aromatic ring, or a substituted or unsubstituted heteroaromatic ring, R¹ to R⁹ each independently represent a hydrogen atom, a deuterium atom or a substituent. R¹ and R², R² and R³, R³ and R⁴, R⁴ and R⁵, R⁵ and R⁶, R⁷ and R⁸, and R⁸ and R⁹ each can bond to each other to form a cyclic structure. However, at least one of the ring formed by Z¹, Z², R¹ and R² bonding to each other, the ring formed by R² and R³ bonding to each other, the ring formed by R⁴ and R⁵ bonding to each other, and the ring formed by R⁵ and R⁶ bonding to each other is a furan ring of a substituted or unsubstituted benzofuran, a thiophene ring of a substituted or unsubstituted benzothiophene, or a pyrrole ring of a substituted or unsubstituted indole, and at least one of R¹ to R⁹ is a substituted or unsubstituted aryl group or an acceptor group, or at least one of Z¹ and Z² is a ring having an aryl group or an acceptor group as a substituent. Of the benzene ring skeleton-constituting carbon atoms to constitute the benzofuran ring, the benzothiophene ring, and the indole ring, a substitutable carbon atom can be substituted with a nitrogen atom. In the general formula (1), C—R¹, C—R², C—R³, C—R⁴, C—R⁵, C—R⁶, C—R⁷, C—R⁸, and C—R⁹ can be substituted with N.

Compounds represented by the following general formula (E4) are further preferred light emitting materials.

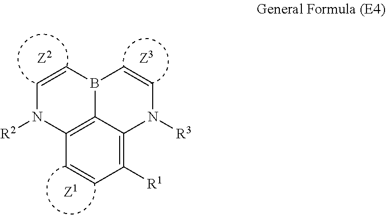

In the general formula (E4), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring, Z² and Z³ each independently represent a substituted or unsubstituted aromatic ring, or a substituted or unsubstituted heteroaromatic ring, R¹ represents a hydrogen atom, a deuterium atom, or a substituent, and R² and R³ each independently represent a substituted or unsubstituted aryl group, or a substituted or unsubstituted heteroaryl group. Z¹ and R¹, R² and Z², Z² and Z³, and Z³ and R³ each can bond to each other to form a cyclic structure. However, at least one pair of R² and Z², Z² and Z³, and Z³ and R³ bonds to each other to form a cyclic structure.

Compounds represented by the following general formula (E5) are further preferred light emitting materials.

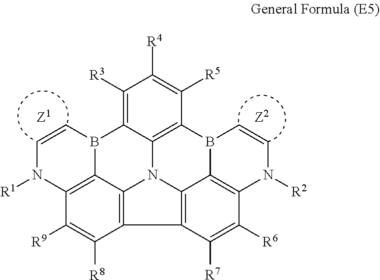

In the general formula (E5), R¹ and R² each independently represent a substituted or unsubstituted alkyl group, a substituted or unsubstituted aryl group, or a substituted or unsubstituted heteroaryl group, Z¹ and Z² each independently represent a substituted or unsubstituted aromatic ring, or a substituted or unsubstituted heteroaromatic ring, and R³ to R⁹ each independently represent a hydrogen atom, a deuterium atom or a substituent. However, at least one of R¹, R², Z¹ and Z² includes a substituted or unsubstituted benzofuran ring, a substituted or unsubstituted benzothiophene ring, or a substituted or unsubstituted indole ring. R¹ and Z¹, Z¹ and R³, R³ and R⁴, R⁴ and R⁵, R⁵ and Z², Z² and R², R² and R⁶, R⁶ and R⁷, R⁷ and R⁸, R⁸ and R⁹, and R⁹ and R¹ each can bond to each other to form a cyclic structure. Of the benzene ring skeleton-constituting carbon atoms to constitute the benzofuran ring, the benzothiophene ring, and the indole ring, a substitutable carbon atom can be substituted with a nitrogen atom. In the general formula (1), C—R³, C—R⁴, C—R⁵, C—R⁶, C—R⁷, C—R⁸, and C—R⁹ can be substituted with N.

Compounds represented by the following general formula (E6) are further preferred light emitting materials.

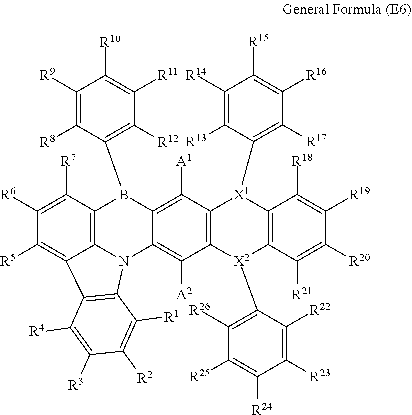

In the general formula (E6), one of X¹ and X² is a nitrogen atom, and the other is a boron atom. R¹ to R²⁶, A¹ and A² each independently represent a hydrogen atom, a deuterium atom, or a substituent. R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ each can bond to each other to form a cyclic structure. However, when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring. However, in the case where X¹ is a nitrogen atom, and where R⁷ and R⁸ and R²¹ and R₂₂ each bond to each other via a nitrogen atom to form a 6-membered ring, and R¹⁷ and R¹⁸ bond to each other to form a single bond, at least one of R¹ to R⁶ is a substituted or unsubstituted aryl group, or any of R¹ and R², R² and R³, R³ and R⁴, R⁴ and R⁵, and R⁵ and R⁶ bond to each other to form an aromatic ring or a heteroaromatic ring.

Compounds represented by the following general formula (E7) are further preferred light emitting materials.

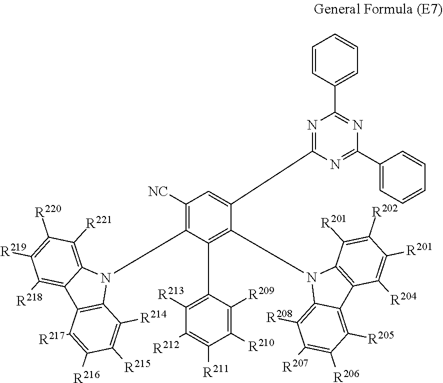

In the general formula (E7), R²⁰¹ to R²²¹ each independently represent a hydrogen atom, a deuterium atom or a substituent, preferably a hydrogen atom, a deuterium atom, an alkyl group, an aryl group, or a group formed by combining an alkyl group and an aryl group. At least one pair of R²⁰¹ and R²⁰², R²⁰² and R²⁰³, R²⁰³ and R²⁰⁴, R²⁰⁵ and R²⁰⁶, R²⁰⁶ and R²⁰⁷, R²⁰⁷ and R²⁰⁸, R²¹⁴ and R²¹⁵, R²¹⁵ and R²¹⁶, R²¹⁶ and R²¹⁷, R²¹⁸ and R²¹⁹, R²¹⁹ and R²²⁰, and R²²⁰ and R²²¹ each bond to each other to form a benzofuro structure or a benzothieno structure. Preferably, one or two pairs of R²⁰¹ and R²⁰², R²⁰² and R²⁰³, R²⁰³ and R²⁰⁴, R²⁰⁵ and R²⁰⁶, R²⁰⁶ and R²⁰⁷ and R²⁰⁷ and R²⁰⁸, and one or two pairs of R²¹⁴ and R²¹⁵, R²¹⁵ and R²¹⁶, R²¹⁶ and R²¹⁷, R²¹⁸ and R²¹⁹, R²¹⁹ and R²²⁰ and R²²⁰ and R²²¹ bond to each other to form a benzofuro structure or a benzothieno structure. Further preferably, R²⁰³ and R²⁰⁴ bond to each other to form a benzofuro structure or a benzothieno structure, and even more preferably, R²⁰³ and R²⁰⁴, and R²¹⁶ and R²¹⁷ each bond to each other to form a benzofuro structure or a benzothieno structure. Especially preferably, R²⁰³ and R²⁰⁴, and R²¹⁶ and R²¹⁷ each bond to each other to form a benzofuro structure or a benzothieno structure, and R²⁰⁶ and R²¹⁹ each represent a substituted or unsubstituted aryl group (preferably, a substituted or unsubstituted phenyl group, and more preferably an unsubstituted phenyl group).

Further, compounds represented by the general formula (1) described in each specification of Japanese Patent Application Nos. 2021-103698, 2021-103699, 2021-103700, 2021-081332, 2021-103701, 2021-151805, and 2021-188860 can be used as a light emitting material. Descriptions of these general formulae (1) and specific compounds are hereby incorporated by reference as a part of this description.

In some embodiments where a host material is used, the amount of the compound of the present invention contained in a light emitting layer as a light emitting material is 0.1% by weight or more. In some embodiments where a host material is used, the amount of the compound of the present invention contained in a light emitting layer as a light emitting material is 1% by weight or more. In some embodiments where a host material is used, the amount of the compound of the present invention contained in a light emitting layer as a light emitting material is 50% by weight or less. In some embodiments where a host material is used, the amount of the compound of the present invention contained in a light emitting layer as a light emitting material is 20% by weight or less. In some embodiments where a host material is used, the amount of the compound of the present invention contained in a light emitting layer as a light emitting material is 10% by weight or less.

In some embodiments, the host material in a light emitting layer is an organic compound having a hole transporting capability and an electron transporting capability. In some embodiments, the host material in a light emitting layer is an organic compound that prevents increase in the wavelength of emitted light. In some embodiments, the host material in a light emitting layer is an organic compound having a high glass transition temperature.

In some embodiments, the host material is selected from the group consisting of the followings:

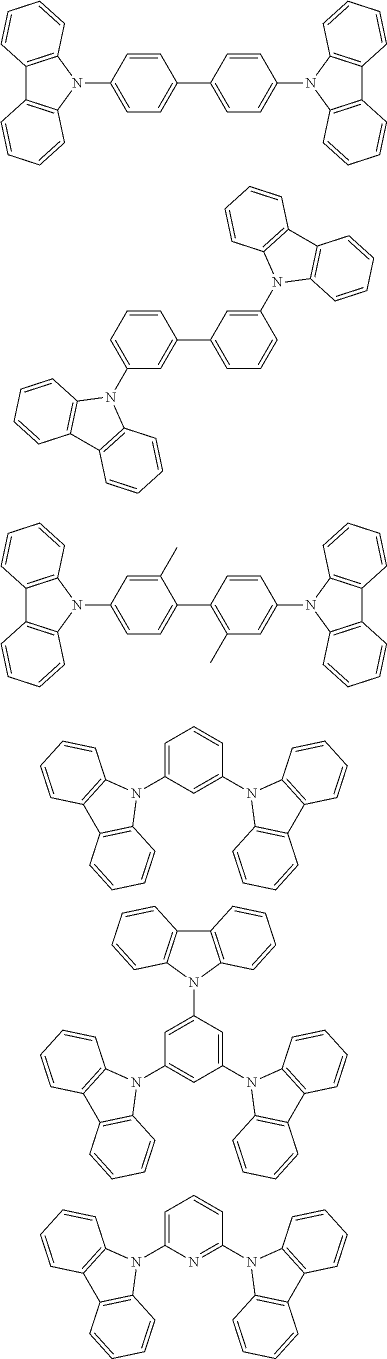

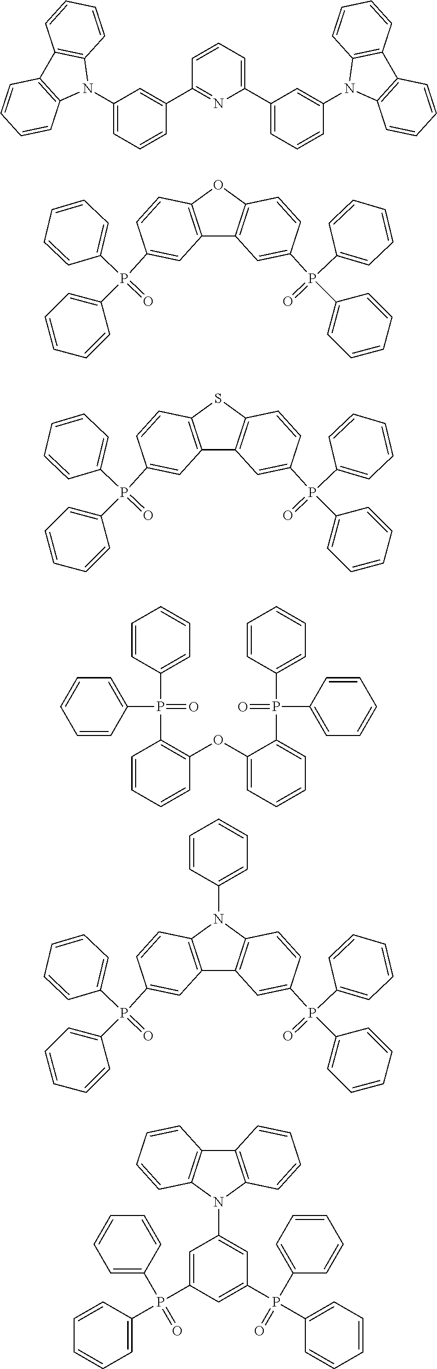

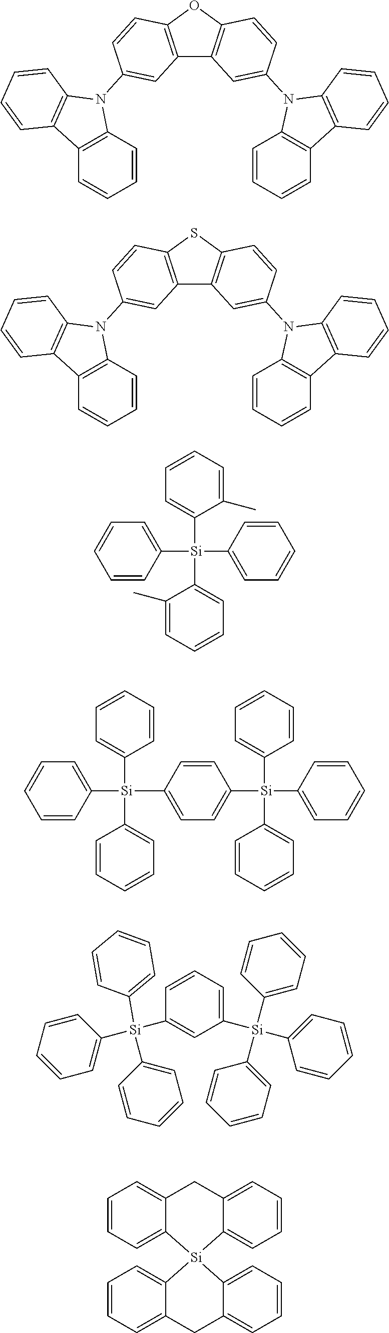

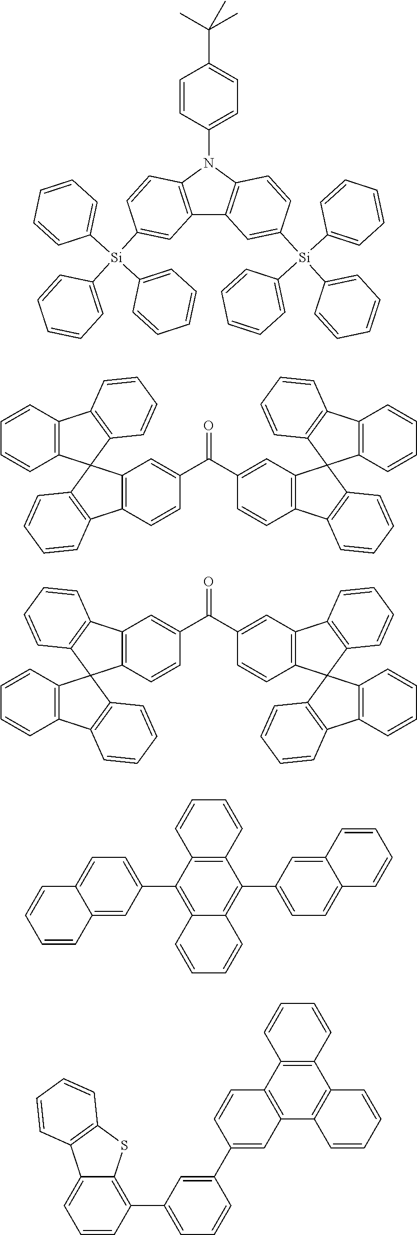

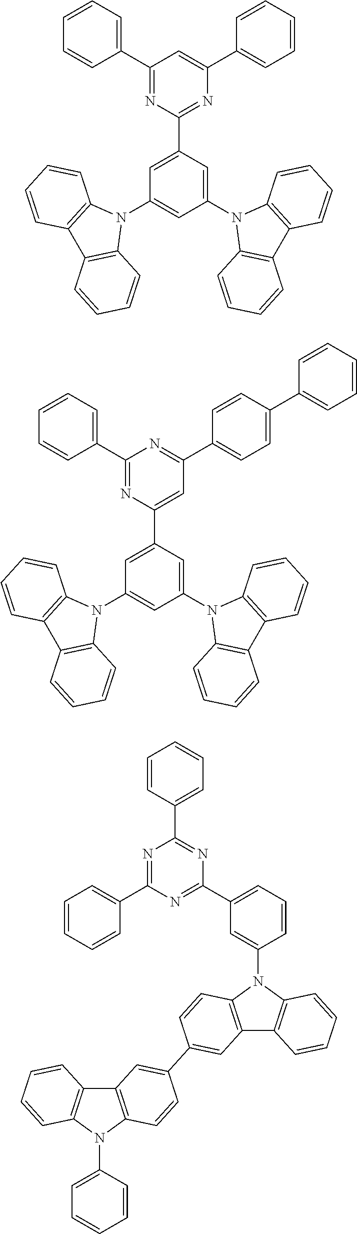 [SMILES: c1ccc(-c2cc(-c3ccccc3)nc(-c3cc(-n4c5ccccc5c5ccccc54)cc(-n4c5ccccc5c5ccccc54)c3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

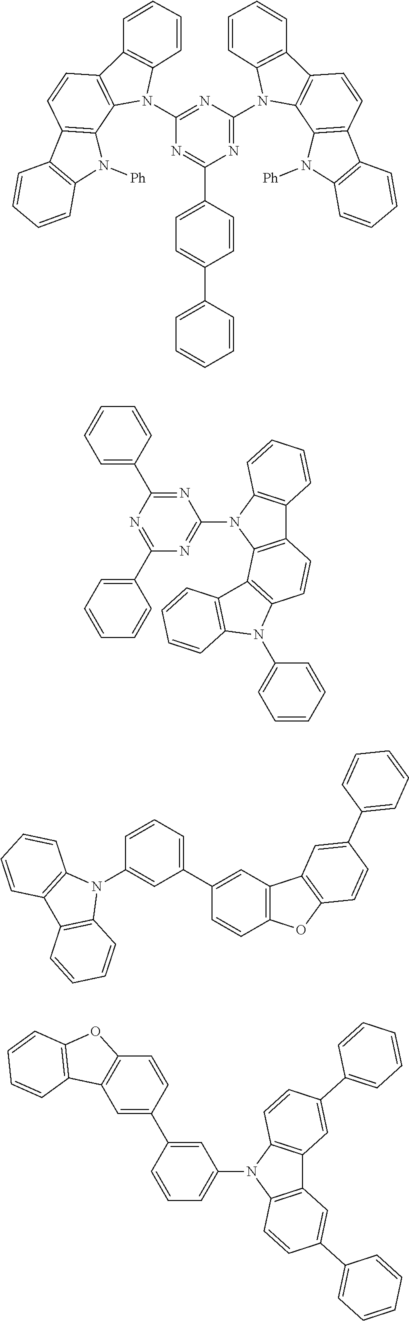 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2ccc3oc4ccc(-c5cccc(-n6c7ccccc7c7ccccc76)c5)cc4c3c2)cc1 ; c1ccc(-c2ccc3c(c2)c2cc(-c4ccccc4)ccc2n3-c2cccc(-c3ccc4oc5ccccc5c4c3)c2)cc1]

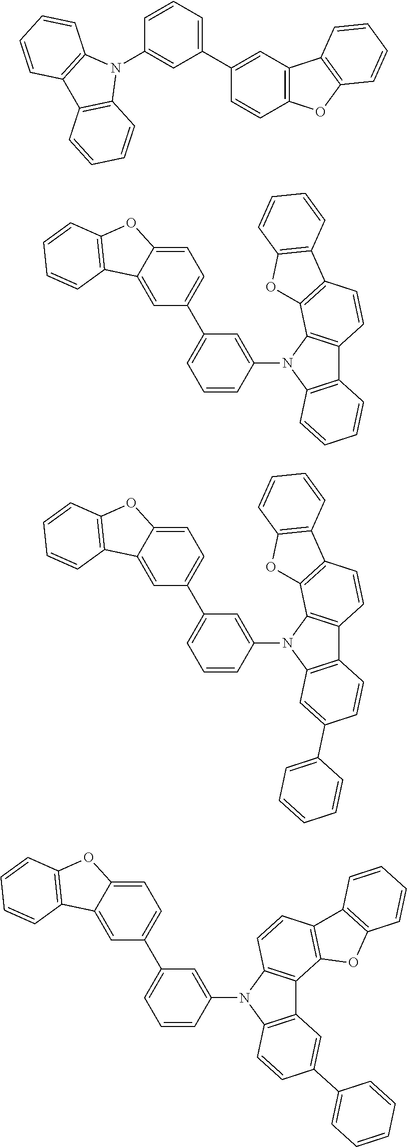

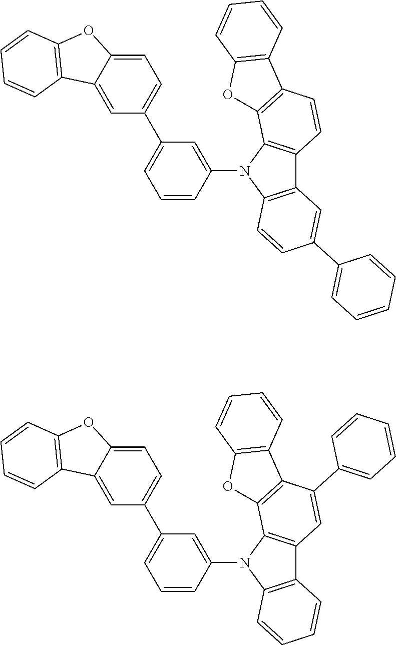 [SMILES: c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

In some embodiments, the light emitting layer contains two or more kinds of TADF molecules differing in the structure. For example, the light emitting layer can contain three kinds of materials of a host material, a first TADF molecule and a second TADF molecule whose excited singlet energy level is higher in that order. In that case, both the first TADF molecule and the second TADF molecule are preferably such that the difference ΔE_{ST} between the lowest excited singlet energy level and the lowest excited triplet energy level at 77 K is 0.3 eV or less, more preferably 0.25 eV or less, even more preferably 0.2 eV or less, further more preferably 0.15 eV or less, further more preferably 0.1 eV or less, further more preferably 0.07 eV or less, further more preferably 0.05 eV or less, further more preferably 0.03 eV or less, and particularly preferably 0.01 eV or less. The content of the first TADF molecule in the light emitting layer is preferably larger than the content of the second TADF molecule therein. The content of the host material in the light emitting layer is preferably larger than the content of the second TADF molecule therein. The content of the first TADF molecule in the light emitting layer can be larger than or can be smaller than or can be the same as the content of the host material therein. In some embodiments, the composition in the light emitting layer can be 10 to 70% by weight of a host material, 10 to 80% by weight of a first TADF molecule, and 0.1 to 30% by weighty of a second TADF molecule. In some embodiments, the composition in the light emitting layer can be 20 to 45% by weight of a host material, 50 to 75% by weight of a first TADF molecule, and 5 to 20% by weighty of a second TADF molecule. In some embodiments, the emission quantum yield φPL1(A) by photo-excitation of a co-deposited film of a first TADF molecule and a host material (the content of the first TADF molecule in the co-deposited film=A % by weight) and the emission quantum yield φPL2(A) by photo-excitation of a co-deposited film of a second TADF molecule and a host material (the content of the second TADF molecule in the co-deposited film=A % by weight) satisfy a relational formula φPL1(A)>φPL2(A). In some embodiments, the emission quantum yield φPL2(B) by photo-excitation of a co-deposited film of a second TADF molecule and a host material (the content of the second TADF molecule in the co-deposited film=B % by weight) and the emission quantum yield φPL2(100) by photo-excitation of a single film of a second TADF molecule satisfy a relational formula φPL2(B)>φPL2(100). In some embodiments, the light emitting layer can contain three kinds of TADF molecules differing in the structure. The compound of the present invention can be any of the plural TADF compounds contained in the light emitting layer.

In some embodiments, the light emitting layer can be composed of materials selected from the group consisting of a host material, an assist dopant and a light emitting material. In some embodiments, the light emitting layer does not contain a metal element. In some embodiments, the light emitting layer can be formed of a material composed of atoms alone selected from the group consisting of a carbon atom, a hydrogen atom, a deuterium atom, a nitrogen atom, an oxygen atom and a sulfur atom. Or the light emitting layer can be formed of a material composed of atoms alone selected from the group consisting of a carbon atom, a hydrogen atom, a deuterium atom, a nitrogen atom and an oxygen atom. Or the light emitting layer can be formed of a material composed of atoms alone selected from the group consisting of a carbon atom, a hydrogen atom, a nitrogen atom and an oxygen atom.

In the case where the light emitting layer contains any other TADF material than the compound of the present invention, the TADF material can be a known delayed fluorescent material. As preferred delayed fluorescent materials, there can be mentioned compounds included in the general formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101 to 0121; JP 2014-9352 A, paragraphs 0007 to 0041 and 0060 to 0069; JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; WO2018/047853, paragraphs 0016 to 0044; and especially, exemplary compounds therein capable of emitting delayed fluorescence. In addition, also preferably employable here are light emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240) A, WO2015/129714, WO2015/129715, WO2015/133501, WO2015/136880, WO2015/137244, WO2015/137202, WO2015/137136, WO2015/146541, and WO2015/159541, These patent publications described in this paragraph are hereby incorporated as a part of this description by reference.

In the following, the constituent members and the other layers than the light emitting layer of the organic electroluminescent device are described.

### Substrate:

In some embodiments, the organic electroluminescent device of the invention is supported by a substrate, wherein the substrate is not particularly limited and can be any of those that have been commonly used in an organic electroluminescent device, for example those formed of glass, transparent plastics, quartz and silicon.

### Anode:

In some embodiments, the anode of the organic electroluminescent device is made of a metal, an alloy, an electroconductive compound, or a combination thereof. In some embodiments, the metal, alloy, or electroconductive compound has a large work function (4 eV or more). In some embodiments, the metal is Au. In some embodiments, the electroconductive transparent material is selected from CuI, indium tin oxide (ITO), SnO₂, and ZnO. In some embodiments, an amorphous material capable of forming a transparent electroconductive film, such as IDIXO (In₂O₃—ZnO), is be used. In some embodiments, the anode is a thin film. In some embodiments, the thin film is made by vapor deposition or sputtering. In some embodiments, the film is patterned by a photolithography method. In some embodiments, where the pattern may not require high accuracy (for example, approximately 100 μm or more), the pattern can be formed with a mask having a desired shape on vapor deposition or sputtering of the electrode material. In some embodiments, when a material can be applied as a coating, such as an organic electroconductive compound, a wet film forming method, such as a printing method or a coating method is used. In some embodiments, when the emitted light goes through the anode, the anode has a transmittance of more than 10%, and the anode has a sheet resistance of several hundred Ohm per unit area or less. In some embodiments, the thickness of the anode is from 10 to 1,000 nm. In some embodiments, the thickness of the anode is from 10 to 200 nm. In some embodiments, the thickness of the anode varies depending on the material used.

### Cathode:

In some embodiments, the cathode is made of an electrode material such as a metal having a small work function (4 eV or less) (referred to as an electron injection metal), an alloy, an electroconductive compound, or a combination thereof. In some embodiments, the electrode material is selected from sodium, a sodium-potassium alloy, magnesium, lithium, a magnesium-copper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth element. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per unit area or less. In some embodiments, the thickness of the cathode ranges from 10 nm to 5 μm. In some embodiments, the thickness of the cathode ranges from 50 to 200 nm. In some embodiments, for transmitting the emitted light, any one of the anode and the cathode of the organic electroluminescent device is transparent or translucent. In some embodiments, the transparent or translucent electroluminescent devices enhances the light emission luminance.

In some embodiments, the cathode is formed with an electroconductive transparent material, as described for the anode, to form a transparent or translucent cathode. In some embodiments, a device comprises an anode and a cathode, both being transparent or translucent.

### Injection Layer;

An injection layer is a layer between the electrode and the organic layer. In some embodiments, the injection layer decreases the drive voltage and enhances the light emission luminance. In some embodiments, the injection layer includes a hole injection layer and an electron injection layer. The injection layer can be positioned between the anode and the light emitting layer or the hole transporting layer, and between the cathode and the light emitting layer or the electron transporting layer. In some embodiments, an injection layer is present. In some embodiments, no injection layer is present.

Preferred compound examples for use as a hole injection material are shown below.

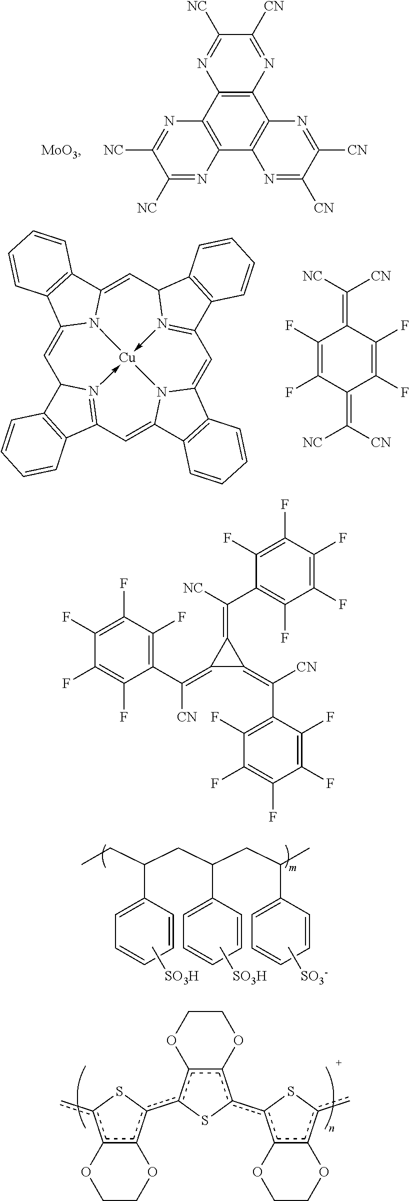

Next, preferred compound examples for use as an electron injection material are shown below.

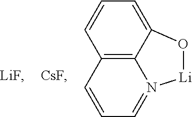

### Barrier Layer:

A barrier layer is a layer capable of inhibiting charges (electrons or holes) and/or excitons present in the light emitting layer from being diffused outside the light emitting layer. In some embodiments, the electron barrier layer is between the light emitting layer and the hole transporting layer, and inhibits electrons from passing through the light emitting layer toward the hole transporting layer. In some embodiments, the hole barrier layer is between the light emitting layer and the electron transporting layer, and inhibits holes from passing through the light emitting layer toward the electron transporting layer. In some embodiments, the barrier layer inhibits excitons from being diffused outside the light emitting layer. In some embodiments, the electron barrier layer and the hole barrier layer are exciton barrier layers. As used herein, the term “electron barrier layer” or “exciton barrier layer” includes a layer that has both the function of an electron barrier layer and the function of an exciton barrier layer.

### Hole Barrier Layer:

A hole barrier layer acts as an electron transporting layer. In some embodiments, the hole barrier layer inhibits holes from reaching the electron transporting layer while transporting electrons. In some embodiments, the hole barrier layer enhances the recombination probability of electrons and holes in the light emitting layer. The material used for the hole barrier layer can be the same materials as the ones described for the electron transporting layer.

Preferred compound examples for use for the hole barrier layer are shown below.

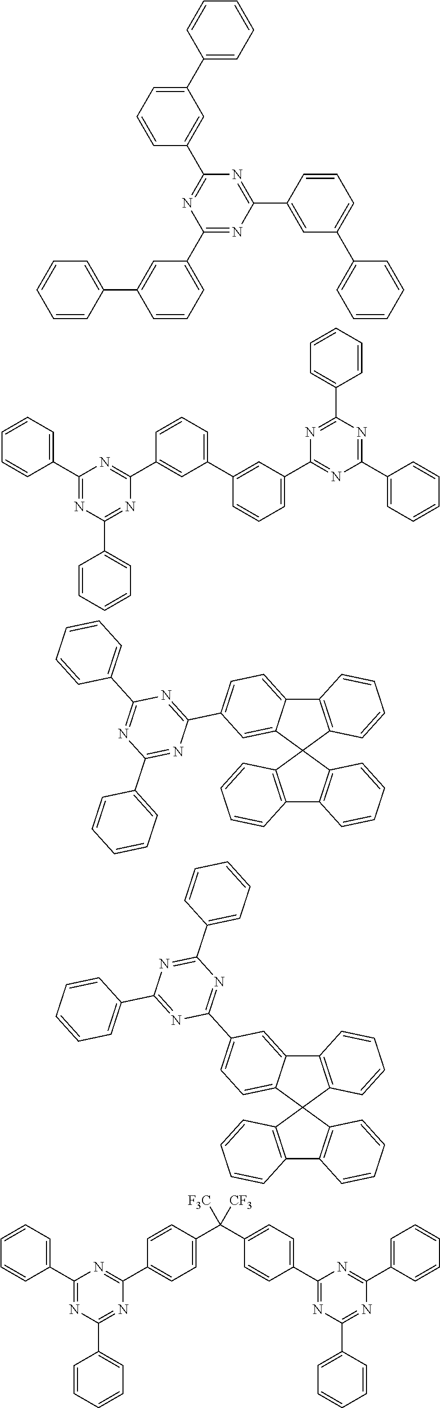

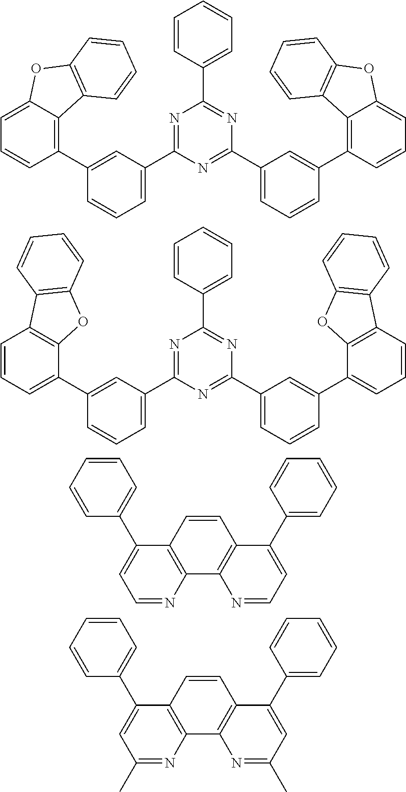

### Electron Barrier Layer:

An electron barrier layer transports holes. In some embodiments, the electron barrier layer inhibits electrons from reaching the hole transporting layer while transporting holes. In some embodiments, the electron barrier layer enhances the recombination probability of electrons and holes in the light emitting layer. The material used for the electron barrier layer can be the same material as the ones described above for the hole transporting layer.

Preferred compound examples for use as the electron barrier material are shown below.

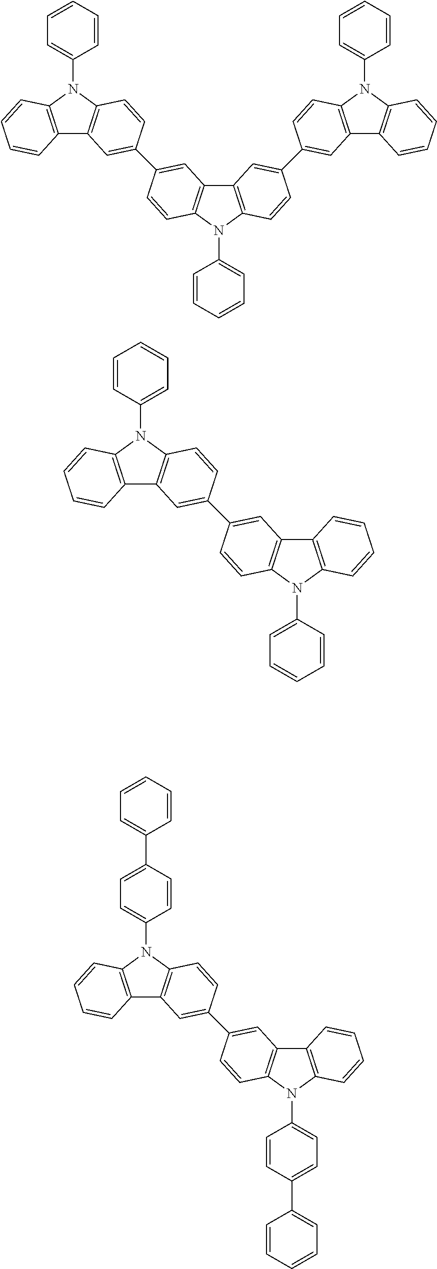 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

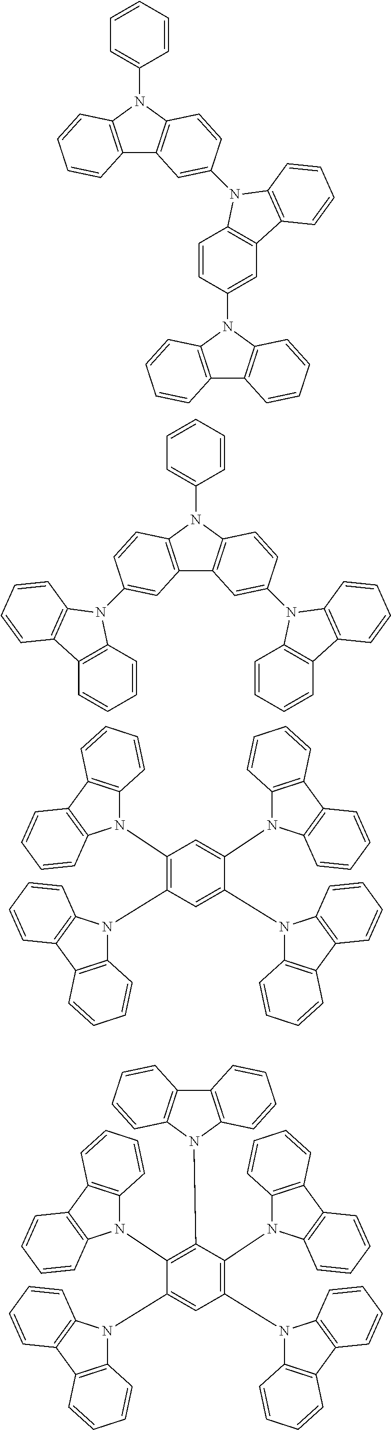

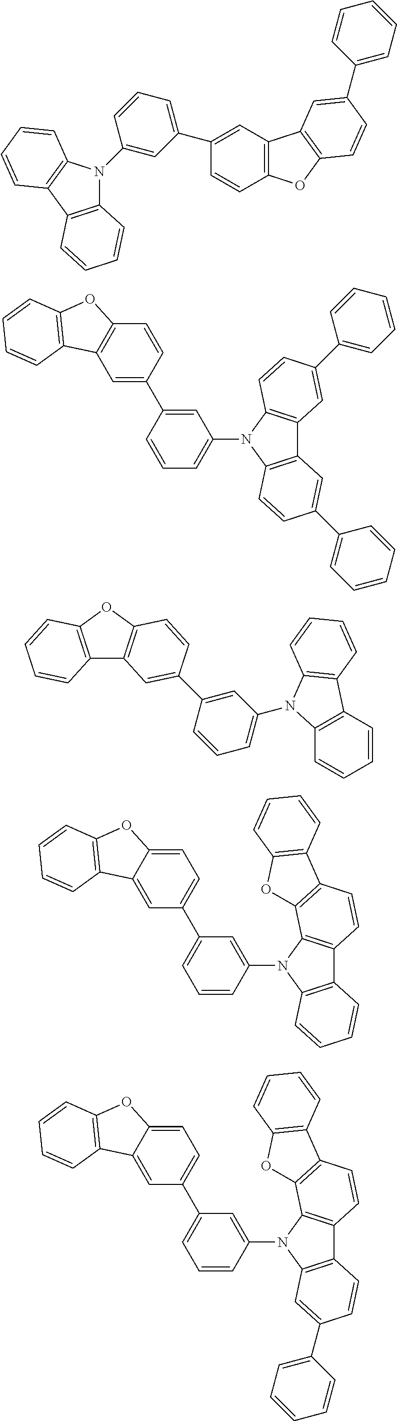

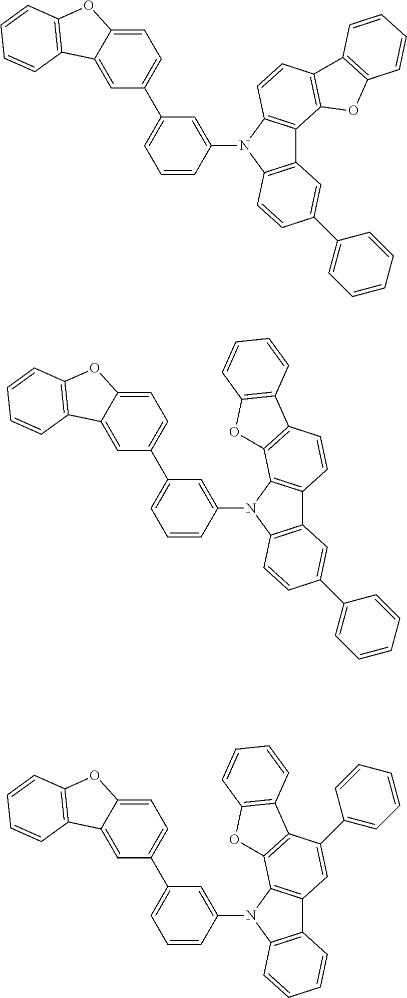 [SMILES: c1ccc(-c2ccc3c(c2)c2c4oc5ccccc5c4ccc2n3-c2cccc(-c3ccc4oc5ccccc5c4c3)c2)cc1 ; c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

### Exciton Barrier Layer:

An exciton barrier layer inhibits excitons generated through recombination of holes and electrons in the light emitting layer from being diffused to the charge transporting layer. In some embodiments, the exciton barrier layer enables effective confinement of excitons in the light emitting layer. In some embodiments, the light emission efficiency of the device is enhanced. In some embodiments, the exciton barrier layer is adjacent to the light emitting layer on any of the side of the anode and the side of the cathode, and on both the sides. In some embodiments, where the exciton barrier layer is on the side of the anode, the layer can be between the hole transporting layer and the light emitting layer and adjacent to the light emitting layer. In some embodiments, where the exciton barrier layer is on the side of the cathode, the layer can be between the light emitting layer and the cathode and adjacent to the light emitting layer. In some embodiments, a hole injection layer, an electron barrier layer, or a similar layer is between the anode and the exciton barrier layer that is adjacent to the light emitting layer on the side of the anode. In some embodiments, a hole injection layer, an electron barrier layer, a hole barrier layer, or a similar layer is between the cathode and the exciton barrier layer that is adjacent to the light emitting layer on the side of the cathode. In some embodiments, the exciton barrier layer comprises excited singlet energy and excited triplet energy, at least one of which is higher than the excited singlet energy and the excited triplet energy of the light emitting material, respectively.

### Hole Transporting Layer:

The hole transporting layer comprises a hole transporting material. In some embodiments, the hole transporting layer is a single layer. In some embodiments, the hole transporting layer comprises a plurality of layers.

In some embodiments, the hole transporting material has one of injection or transporting property of holes and barrier property of electrons. In some embodiments, the hole transporting material is an organic material. In some embodiments, the hole transporting material is an inorganic material. Examples of known hole transporting materials that can be used in the present invention include but are not limited to a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an allylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer (particularly, a thiophene oligomer), or a combination thereof. In some embodiments, the hole transporting material is selected from a porphyrin compound, an aromatic tertiary amine compound, and a styrylamine compound. In some embodiments, the hole transporting material is an aromatic tertiary amine compound. Preferred compound examples for use as the hole transporting material are shown below.

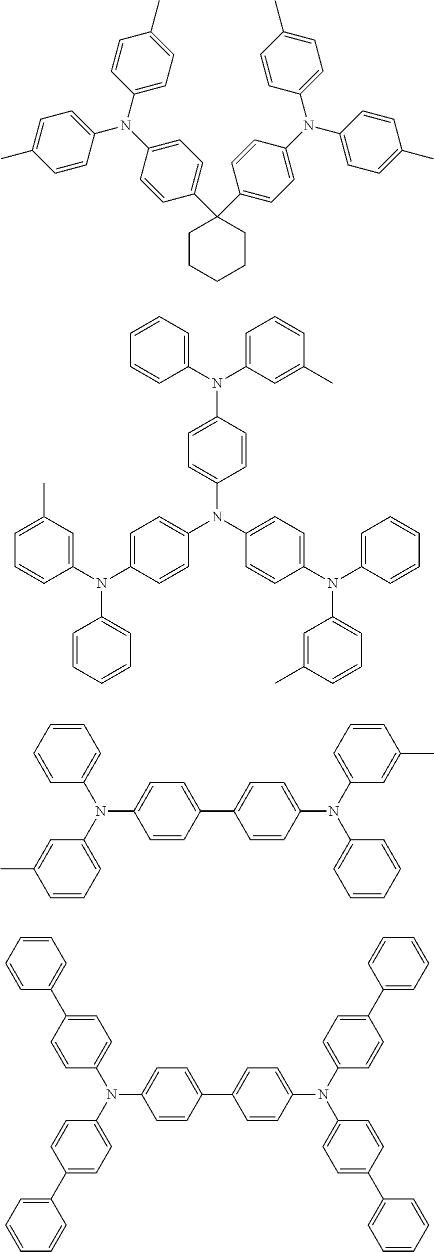

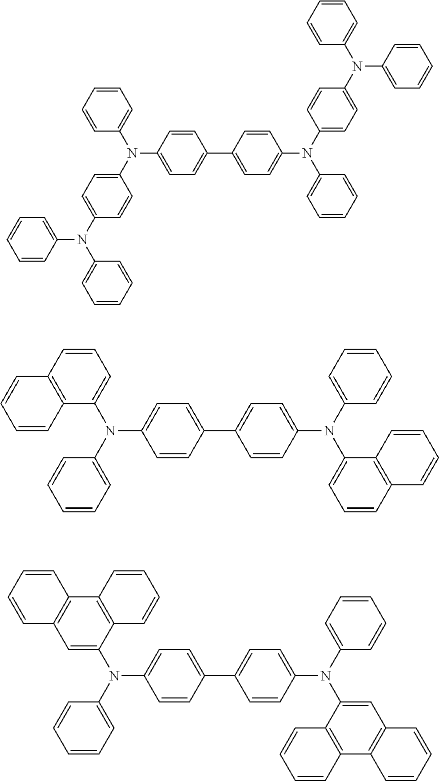 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(N(c2ccc(-c3ccc(N(c4ccccc4)c4cc5ccccc5c5ccccc45)cc3)cc2)c2cc3ccccc3c3ccccc23)cc1]

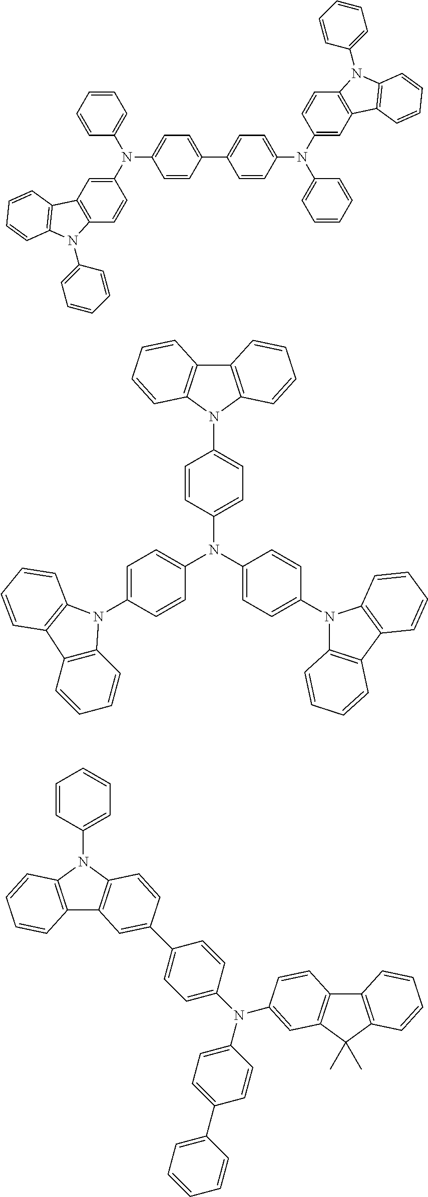 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

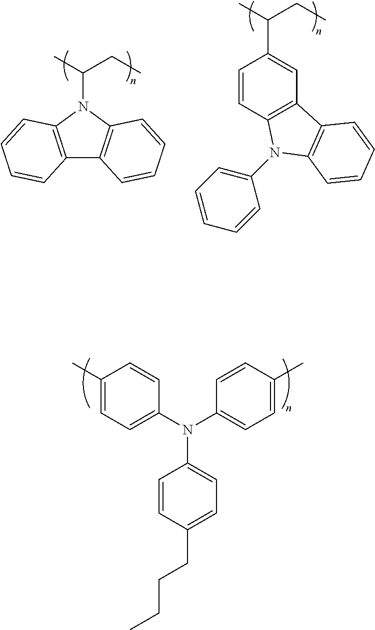

### Electron Transporting Layer:

The electron transporting layer comprises an electron transporting material. In some embodiments, the electron transporting layer is a single layer. In some embodiments, the electron transporting layer comprises a plurality of layers.

In some embodiments, the electron transporting material needs only to have a function of transporting electrons, which are injected from the cathode, to the light emitting layer. In some embodiments, the electron transporting material also function as a hole barrier material. Examples of the electron transporting layer that can be used in the present invention include but are not limited to a nitro-substituted fluorene derivative, a diphenylquinone derivative, a thiopyran dioxide derivative, carbodiimide, a fluorenylidene methane derivative, anthraquinodimethane, an anthrone derivatives, an oxadiazole derivative, an azole derivative, an azine derivative, or a combination thereof, or a polymer thereof. In some embodiments, the electron transporting material is a thiadiazole derivative, or a quinoxaline derivative. In some embodiments, the electron transporting material is a polymer material. Preferred compound examples for use as the electron transporting material are shown below.

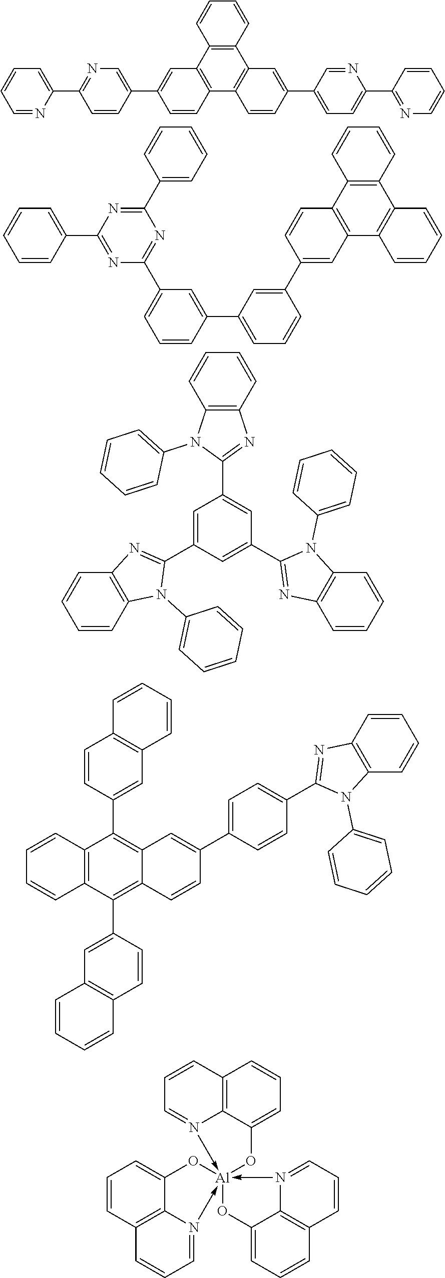

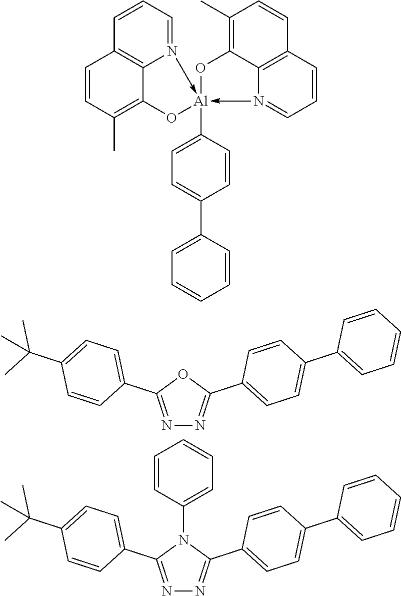

Hereinunder, compound examples preferred as a material that can be added to the organic layers are shown. For example, it is conceivable to add these as a stabilization material.

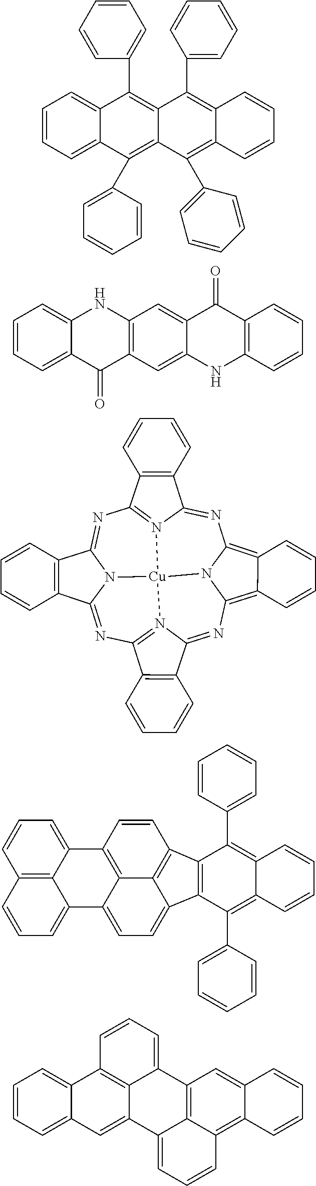

Preferred materials for use in the organic electroluminescent device are specifically shown. However, the materials usable in the invention should not be limitatively interpreted by the following exemplary compounds. Compounds that are exemplified as materials having a specific function can also be used as materials having any other function.

### Devices:

In some embodiments, an light emitting layer is incorporated into a device. For example, the device includes, but is not limited to an OLED bulb, an OLED lamp, a television screen, a computer monitor, a mobile phone, and a tablet.

In some embodiments, an electronic device includes an OLED comprising an anode, a cathode, and at least one organic layer comprising a light emitting layer between the anode and the cathode.

In some embodiments, compositions described herein can be incorporated into various light-sensitive or light-activated devices, such as OLEDs or opto-electronic devices. In some embodiments, the composition can be useful in facilitating charge transfer or energy transfer within a device and/or as a hole-transport material. The device can be, for example, an organic light emitting diode (OLED), an organic integrated circuit (O-IC), an organic field-effect transistor (O-FET), an organic thin-film transistor (O-TFT), an organic light emitting transistor (O-LET), an organic solar cell (O-SC), an organic optical detector, an organic photoreceptor, an organic field-quench device (O-FQD), a light-emitting electrochemical cell (LEC) or an organic laser diode (O-laser).

### Bulbs or Lamps:

In some embodiments, an electronic device includes an OLED comprising an anode, a cathode, and at least one organic layer comprising a light emitting layer between the anode and the cathode.

In some embodiments, a device comprises OLEDs that differ in color. In some embodiments, a device comprises an array comprising a combination of OLEDs. In some embodiments, the combination of OLEDs is a combination of three colors (e.g., RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light comprising:
a circuit board having a first surface with a mounting surface and an opposing second surface, and defining at least one opening;
at least one OLED on the mounting surface, the at least one OLED configured to emanate light, the OLED comprising: an anode, a cathode, and at least one organic layer comprising a light emitting layer between the anode and the cathode;
a housing for the circuit board; and
at least one connector arranged at an end of the housing, the housing and the connector defining a package adapted for installation in a light fixture.

In some embodiments, the OLED light comprises a plurality of OLEDs mounted on a circuit board such that light emanates in a plurality of directions. In some embodiments, a portion of the light emanated in a first direction is deflected to emanate in a second direction. In some embodiments, a reflector is used to deflect the light emanated in a first direction.

### Displays or Screens:

In some embodiments, the light emitting layer of the invention can be used in a screen or a display. In some embodiments, the compounds of the invention are deposited onto a substrate using a process including, but not limited to, vacuum evaporation, deposition, vapor deposition, or chemical vapor deposition (CVD). In some embodiments, the substrate is a photoplate structure useful in a two-sided etching that provides a unique aspect ratio pixel. The screen (which can also be referred to as a mask) is used in a process in the manufacturing of OLED displays. The corresponding artwork pattern design facilitates a very steep and narrow tie-bar between the pixels in the vertical direction and a large, sweeping bevel opening in the horizontal direction. This allows the fine patterning of pixels needed for high resolution displays while optimizing the chemical vapor deposition onto a TFT backplane.

The internal patterning of the pixel allows the construction of a three-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point the entire pixel area is subjected to a similar etching rate but the depths are varying depending on the halftone pattern. Varying the size and spacing of the halftone pattern allow etching to be inhibited at different rates within the pixel and allow a localized deeper etch needed to create steep vertical bevels.

A preferred material for the deposition mask is invar. Invar is a metal alloy that is cold rolled into a long thin sheet in a steel mill. Invar cannot be electrodeposited onto a rotating mandrel as the nickel mask. A preferred and more cost feasible method for forming the opening areas in the mask used for deposition is through a wet chemical etching.

In some embodiments, a screen or display pattern is a pixel matrix on a substrate. In some embodiments, a screen or display pattern is fabricated using lithography (e.g., photolithography and e-beam lithography). In some embodiments, a screen or display pattern is fabricated using a wet chemical etching. In further embodiments, a screen or display pattern is fabricated using plasma etching.

### Methods of Manufacturing Devices:

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

In another aspect, provided herein is a method of manufacturing an organic light emitting diode (OLED) display, the method comprising:
forming a barrier layer on a base substrate of a mother panel;
forming a plurality of display units in units of cell panels on the barrier layer;
forming an encapsulation layer on each of the display units of the cell panels;
applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light emitting layer, a gate electrode, and a source/drain electrode. Each of the plurality of display units may include a thin film transistor (TFT) layer, a planarization film formed on the TFT layer, and a light emitting unit formed on the planarization film, wherein the organic film applied to the interface portion is formed of a same material as a material of the planarization film and is formed at a same time as the planarization film is formed. In some embodiments, a light emitting unit is connected to the TFT layer with a passivation layer and a planarization film therebetween and an encapsulation layer that covers and protects the light emitting unit. In some embodiments of the method of manufacturing, the organic film contacts neither the display units nor the encapsulation layer.

Each of the organic film and the planarization film can include any one of polyimide and acryl. In some embodiments, the barrier layer can be an inorganic film. In some embodiments, the base substrate can be formed of polyimide. The method can further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is manufactured. In some embodiments, the organic film can be formed on the edge portion of the barrier layer such that a portion of the organic film directly contacts the base substrate and a remaining portion of the organic film contacts the barrier layer while surrounding the edge portion of the barrier layer.

In some embodiments, the light emitting layer includes a pixel electrode, a counter electrode, and an organic light emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light emitting layer emits light, thereby forming an image. Hereinafter, an image forming unit including the TFT layer and the light emitting unit is referred to as a display unit.

In some embodiments, the encapsulation layer that covers the display unit and prevents penetration of external moisture can be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding an edge portion of the barrier layer.

In one embodiment, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In one embodiment, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the method of manufacture further comprises cutting along the interface portion, wherein a groove is formed in the barrier layer, wherein at least a portion of the organic film is formed in the groove, and wherein the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is entirely exposed without the organic film, an impact generated when each of the cell panels is cut along the groove at the interface portion is transferred to the barrier layer, thereby increasing the risk of cracks. However, in one embodiment, since the groove at the interface portion between the barrier layers is covered with the organic film and the organic film absorbs an impact that would otherwise be transferred to the barrier layer, each of the cell panels can be softly cut and cracks can be prevented from occurring in the barrier layer. In one embodiment, the organic film covering the groove at the interface portion and the planarization film are spaced apart from each other. For example, if the organic film and the planarization film are connected to each other as one layer, since external moisture may penetrate into the display unit through the planarization film and a portion where the organic film remains, the organic film and the planarization film are spaced apart from each other such that the organic film is spaced apart from the display unit.

In some embodiments, the display unit is formed by forming the light emitting unit, and the encapsulation layer is disposed on the display unit to cover the display unit. As such, once the mother panel is completely manufactured, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks can be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### EXAMPLES

The features of the present invention will be described more specifically with reference to Synthesis Examples and Examples given below. The materials, processes, procedures and the like shown below can be appropriately modified unless they deviate from the substance of the invention. Accordingly, the scope of the invention is not construed as being limited to the specific examples shown below. Hereunder, the light emission characteristics were evaluated using a source meter (available from Keithley Instruments, Inc.: 2400 series), a semiconductor parameter analyzer (available from Agilent Technologies, Inc., E5273A), an optical power meter device (available from Newport Corporation, 1930C), an optical spectroscope (available from Ocean Optics Corporation, USB2000), a spectroradiometer (available from Topcon Corporation, SR-3), and a streak camera (available from Hamamatsu Photonics K.K., Model C4334). The energies of HOMO and LUMO were measured by photoelectron spectroscopy in air (such as AC-3 manufactured by Riken Keiki Co., Ltd.).

In the following Synthesis Examples, compounds included in the general formula (1) were synthesized.

### (Synthesis Example 1) Synthesis of Compound 1

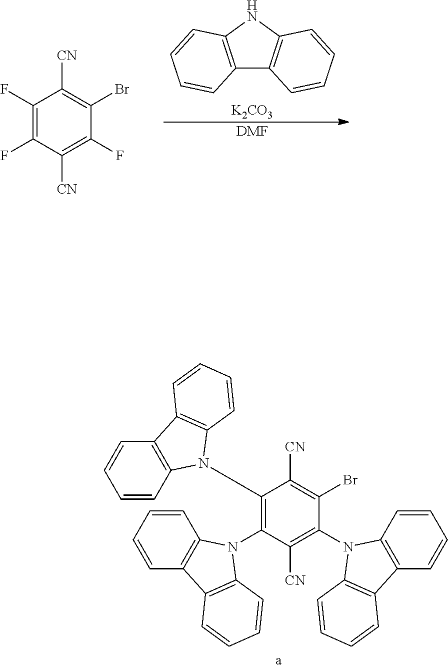

Under a nitrogen stream, an N,N-dimethylformamide solution (120 mL) of 2-bromo-3,5,6-trifluorotetranitrile (1.1 g, 4.14 mmol), 9H-carbazole (2.1 g, 12.6 mmol) and potassium carbonate (2.9 g, 20.7 mmol) was stirred at room temperature for 16 hours. Thereafter, water and methanol were put thereinto to precipitate a solid, and the solid was recovered by filtration. The recovered reaction mixture was purified by silica gel column chromatography (toluene/hexane=4/1) and reprecipitation (toluene/hexane) to give Compound a (1.3 g, 1.9 mmol, yield 45%).

¹H NMR (400 MHz, CDCl₃) δ 8.24-8.20 (m, 2H), 7.72-7.57 (m, 6H), 7.47-7.44 (m, 2H), 7.33-7.29 (m, 2H), 7.13-6.93 (m, 12H).

ASAP Mass Spectrometry; theoretical value 701.12, observed value 702.02.

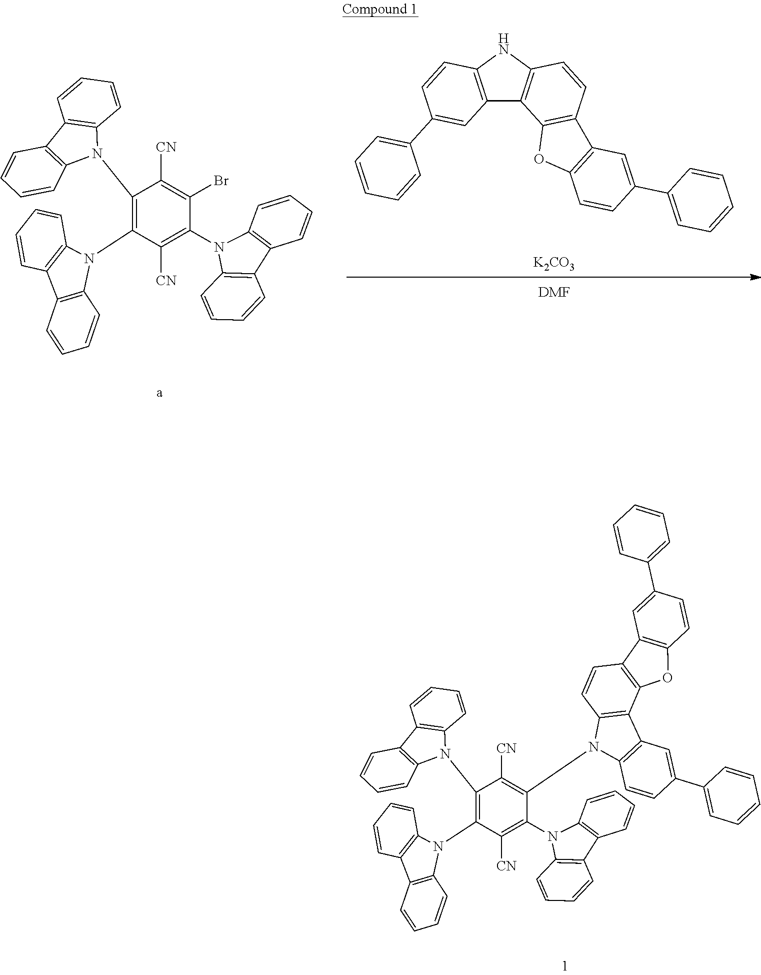

Under a nitrogen stream, an N,N-dimethylformamide solution (30 mL) of Compound a (1.0 g, 1.4 mmol), 2,8-diphenyl-5H-benzofuro[3,2-c]carbazole (0.8 g, 1.9 mmol) and potassium carbonate (0.3 g, 2.2 mmol) was stirred under heat at 120° C. for 16 hours. Thereafter, this was restored to room temperature, then water and methanol were added thereto to precipitate a solid, and the solid was recovered by filtration. The recovered reaction mixture was purified by silica gel column chromatography (toluene/hexane=5/1) and reprecipitation (toluene/methanol) to give Compound 1 (1.0 g, 1.0 mmol, yield 68%).

¹H NMR (400 MHz, CDCl₃) δ 8.39 (s, 1H), 8.15 (s, 1H), 7.82 (d, J=8.8 Hz, 1H), 7.77-7.67 (m, 13H), 7.54-7.49 (m, 4H), 7.44-7.28 (m, 10H), 7.22-7.14 (m, 10H), 7.10-7.07 (m, 2H).

ASAP Mass Spectrometry: theoretical value 1030.34, observed value 1031.11.

### (Synthesis Example 2) Synthesis of Compound 23722

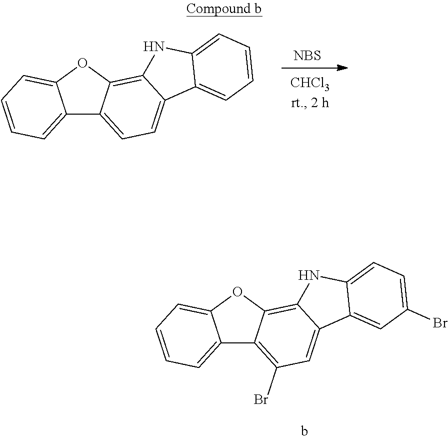

Under a nitrogen atmosphere, 12H-[1]benzofuro[2,3-a]carbazole (4.10 g, 15.9 mmol), N-bromosuccinimide (5.67 g, 31.9 mmol) and chloroform (80 mL) were put into a 200-mL three-neck flask, and stirred at room temperature for 2 hours. After stirred, water was added to the reaction solution for filtration and extraction. The resultant reaction mixture was purified by silica gel column chromatography to give Compound b (4.00 g, 9.64 mmol, yield 60%).

¹H NMR (400 MHz, CDCl₃) δ 8.61 (dq, J=7.8, 0.7 Hz, 2H), 8.20-8.18 (m, 1H), 8.11 (d, J=0.7 Hz, 1H), 7.65 (dt, J=8.4, 0.7 Hz, 1H), 7.57-7.51 (m, 2H), 7.47-7.41 (m, 2H)

ASAP Mass Spectrometry: theoretical value 412.91, observed value 414.91.

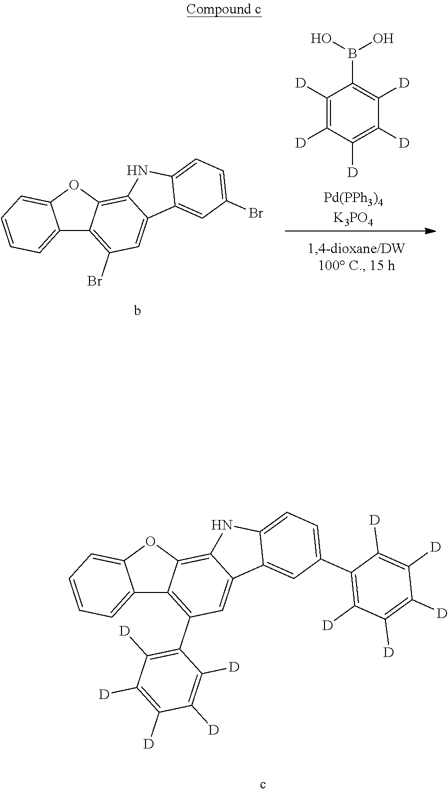 [SMILES: Brc1ccc2[nH]c3c(cc(Br)c4c5ccccc5oc34)c2c1 ; [2H]c1c([2H])c([2H])c(B(O)O)c([2H])c1[2H] ; "1,4-dioxane/DW100° C., 15 h" ; [2H]c1c([2H])c([2H])c(-c2ccc3[nH]c4c(cc(-c5c([2H])c([2H])c([2H])c([2H])c5[2H])c5c6ccccc6oc45)c3c2)c([2H])c1[2H] ; "Pd(PPh3)4K3PO4"]

Under a nitrogen atmosphere, compound b (4.00 g, 9.64 mmol), phenyl-d5-boronic acid (2.94 g, 23.1 mmol), tetrakis(triphenylphosphine) palladium (0) (0.835 g, 0.723 mmol), tripotassium phosphate (6.14 g, 28.9 mmol) and a mixed solvent of 1,4-dioxane (90 mL)/water (30 mL) were put into a 300-mL three-neck flask, and stirred at 100° C. for 15 hours. After stirred, the reaction solution was cooled down to room temperature, and water was added thereto for filtration and extraction. The resultant reaction mixture was purified by silica gel column chromatography to give compound c (2.56 g, 6.10 mmol, yield 63%).

¹H NMR (400 MHz, CDCl₃) δ 8.58 (s, 1H), 8.34 (t, J=0.9 Hz, 1H), 7.99 (d, J=0.5 Hz, 1H), 7.74 (dd, J=8.5, 1.8 Hz, 1H), 7.67-7.62 (m, 2H), 7.57-7.52 (m, 1H), 7.44-7.40 (m, 1H), 7.19-7.15 (m, 1H)

ASAP Mass Spectrometry: theoretical value 419.21, observed value 420.37.

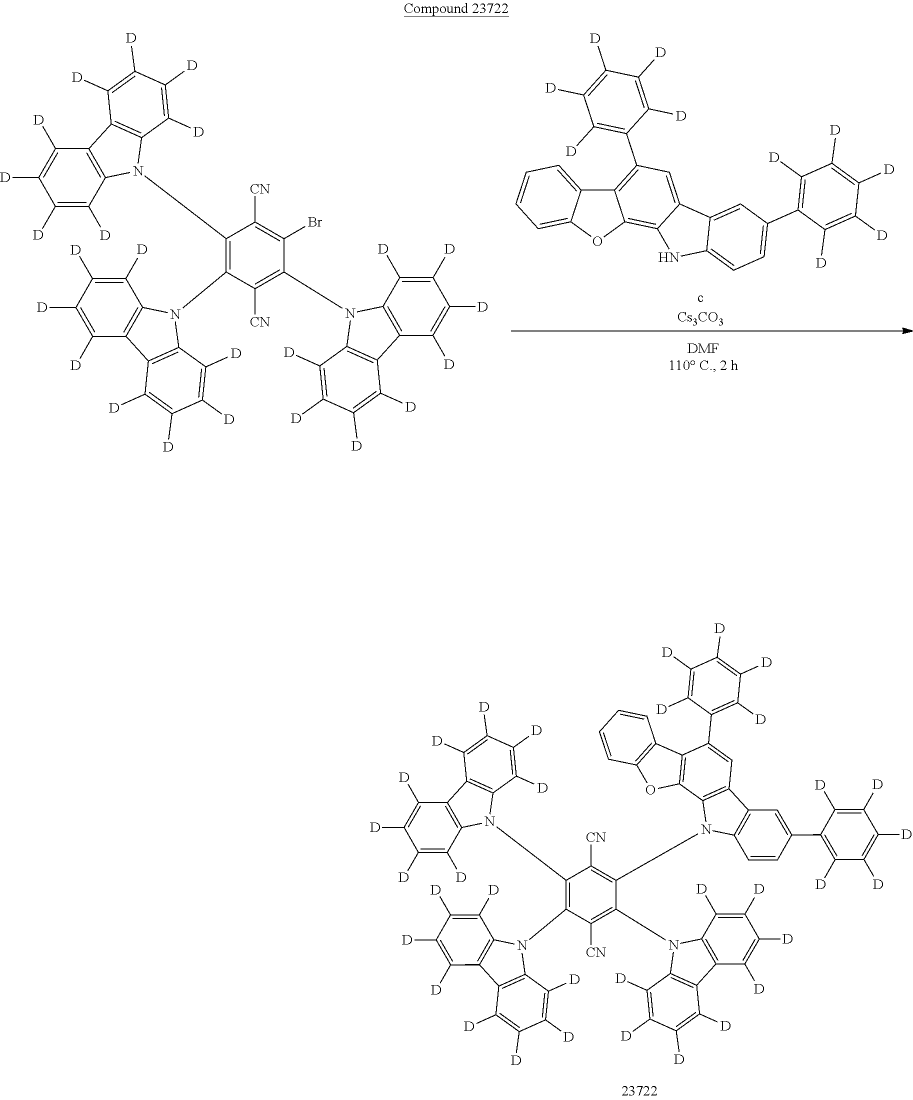

Under a nitrogen atmosphere, 2-bromo-3,5,6-tris(9H-carbazol-9-yl-d8) terephthalonitrile (0.922 g, 1.27 mmol), Compound c (0.798 g, 1.90 mmol), cesium carbonate (0.579 g, 1.78 mmol) and dimethylformamide (20 mL) were put into a 100-ml three-neck flask, then stirred at 110° C. for 2 hours, and thereafter cooled down to room temperature. Water was added to the reaction solution, the precipitated solid was recovered by filtration, and washed with methanol. The resultant solid was purified by silica gel column chromatography to give Compound 23722 (0.82 g, 0.77 mmol, yield 61%).

¹H NMR (400 MHz, CDCl₃) δ 8.01 (d, J=8.0 Hz, 1H), 7.90 (s, 1H), 7.73-7.68 (m, 2H), 7.63 (d, J=7.8 Hz, 1H), 7.36 (t, J=7.4 Hz, 1H), 7.03 (dd, J=13.6, 8.6 Hz, 2H)

ASAP Mass Spectrometry: theoretical value 1064.56, observed value 1066.16.

### (Synthesis Example 3) Synthesis of Compound 23764

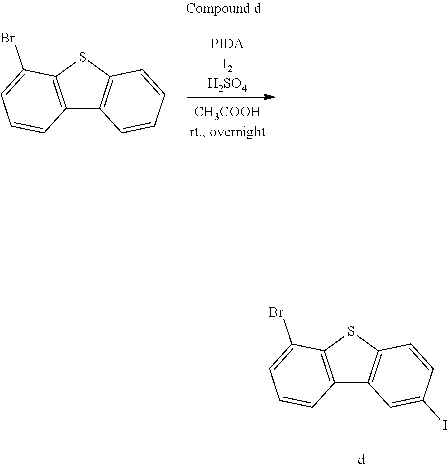

Under a nitrogen atmosphere, acetic acid (880 mL) and concentrated sulfuric acid (0.1 ml) were sequentially added to a mixture of 4-bromodibenzothiophene (38.0 g, 144 mmol), (diacetoxyiodo)benzene (32.6 g, 101 mmol) and iodine (25.7 g, 101 mmol), and reacted overnight. An aqueous saturated sodium hydrogen sulfite solution (50 ml) was added to the reaction solution to stop the reaction, then pure water was added, and the precipitated solid was recovered by filtration and washed with pure water. Dichloromethane and an aqueous solution of sodium hydrogen carbonate were added to the resultant solid for extraction, which was then dried over anhydrous magnesium sulfate. The solution was filtered through a Celite/silica gel pad, and the solvent was evaporated away to give Compound d (40.5 g, 104 mmol, yield 72.1%).

¹H NMR (400 MHz, CDCl₃) δ 8.44 (d, J=1.7 Hz, 1H), 8.05 (dd, J=8.0, 0.9 Hz, 1H), 7.76 (dd, J=8.0, 1.7 Hz, 1H), 7.66-7.61 (m, 2H), 7.36 (t, J=8.0 Hz, 1H).

ASAP Mass Spectrometry: theoretical value 389.05, observed value 389.96.

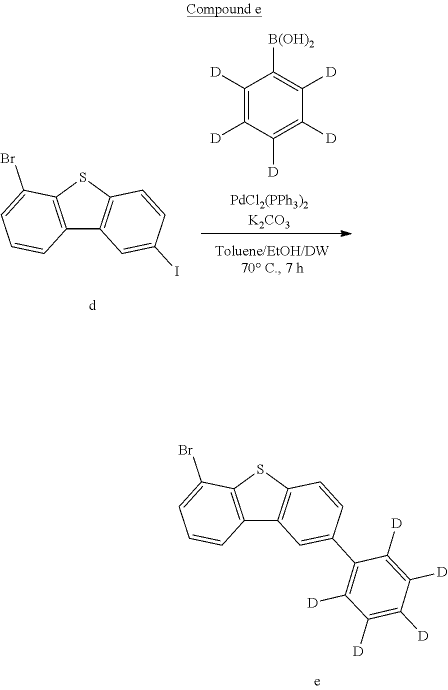 [SMILES: Brc1cccc2c1sc1ccc(I)cc12 ; [2H]c1c([2H])c([2H])c(B(O)O)c([2H])c1[2H] ; "Toluene/EtOH/DW70° C., 7 h" ; [2H]c1c([2H])c([2H])c(-c2ccc3sc4c(Br)cccc4c3c2)c([2H])c1[2H] ; "PdCl2(PPh3)2K2CO3"]

Under a nitrogen atmosphere, a mixed solvent of deaerated toluene (70 mL)/ethanol (10 mL)/water (20 mL) was added to a mixture of compound d (14.5 g, 37.2 mmol), phenyl-d5-boronic acid (4.73 g, 37.2 mmol), dichlorobis(triphenylphosphine) palladium (1.31 g, 1.86 mmol) and potassium carbonate (10.3 g, 74.4 mmol), and reacted at 70° C. for 7 hours. After the reaction, the reaction solution was cooled down to room temperature and extracted. The resultant reaction mixture was purified by silica gel column chromatography to give compound e (9.9 g, 28.8 mmol, yield 93.9%).

¹H NMR (400 MHz, CDCl₃) δ 8.30 (d, J=1.8 Hz, 1H), 8.18 (dd, J=7.9, 1.0 Hz, 1H), 7.95 (dd, J=8.2, 0.7 Hz, 1H), 7.74 (dd, J=8.2, 1.8 Hz, 1H), 7.64 (dd, J=7.8, 0.9 Hz, 1H), 7.36 (t, J=7.8 Hz, 1H).

ASAP Mass Spectrometry: theoretical value 344.28, observed value 345.11.

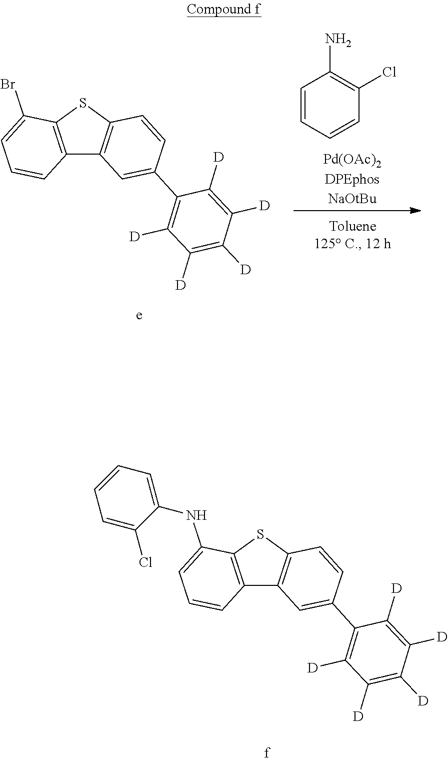 [SMILES: [2H]c1c([2H])c([2H])c(-c2ccc3sc4c(Br)cccc4c3c2)c([2H])c1[2H] ; Nc1ccccc1Cl ; "Pd(OAc)2DPEphosNaOtBu" ; "Toluene125° C., 12 h" ; [2H]c1c([2H])c([2H])c(-c2ccc3sc4c(Nc5ccccc5Cl)cccc4c3c2)c([2H])c1[2H]]

Under a nitrogen atmosphere, deaerated toluene (100 mL) and 2-chloroaniline (3.30 ml, 31.6 mmol) were sequentially added to a mixture of compound e (9.90 g, 28.8 mmol), palladium acetate (0.646 g, 2.88 mmol),bis[2-(diphenylphosphino)phenyl] ether (2.32 g, 4.31 mmol) and sodium tert-butoxide (5.53 g, 57.5 mmol), and reacted at 125° C. for 12 hours. After the reaction, the reaction solution was cooled down to room temperature, and filtered through a Celite/silica gel pad. The resultant reaction mixture was purified by silica gel column chromatography to give compound f (8.10 g, 20.7 mmol, yield 72.1%).

¹H NMR (400 MHz, CDCl₃) δ 8.36 (d, J=1.8 Hz, 1H), 8.01 (dd, J=7.8, 0.9 Hz, 1H), 7.92 (dd, J=8.7, 0.9 Hz, 1H), 7.72 (dd, J=8.2, 1.8 Hz, 1H), 7.48 (t, J=7.8 Hz, 1H), 7.44-7.39 (m, 2H), 7.16-7.04 (m, 2H), 6.88-6.82 (m, 1H), 6.19 (s, 1H).

ASAP Mass Spectrometry: theoretical value 390.94, observed value 391.19.

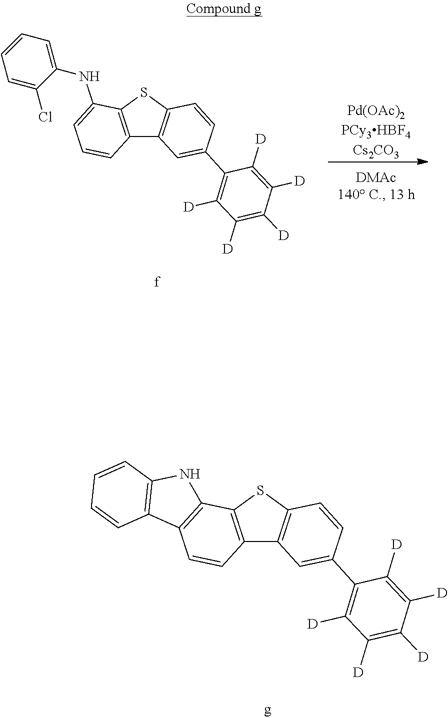 [SMILES: [2H]c1c([2H])c([2H])c(-c2ccc3sc4c(Nc5ccccc5Cl)cccc4c3c2)c([2H])c1[2H] ; "Pd(OAc)2PCy3·HBF4Cs2CO3" ; "DMAc140° C., 13 h" ; [2H]c1c([2H])c([2H])c(-c2ccc3sc4c(ccc5c6ccccc6[nH]c54)c3c2)c([2H])c1[2H]]

Under a nitrogen atmosphere, dimethylacetamide (80 mL) was added to a mixture of compound f (8.10 g, 20.7 mmol), palladium acetate (0.466 g, 2.07 mmol), tricyclohexylphosphonium tetrafluoroborate (1.53 g, 4.15 mmol) and cesium carbonate (20.3 g, 62.2 mmol), and reacted at 140° C. for 13 hours. After the reaction, the reaction solution was cooled down to room temperature, and filtered through a Celite/silica gel pad. The resultant reaction mixture was purified by silica gel column chromatography to give compound g (8.10 g, 20.7 mmol, yield 72.1%).

¹H NMR (400 MHz, CDCl₃) δ 8.45 (d, J=1.8 Hz, 1H), 8.32 (s, 1H), 8.22-8.09 (m, 3H), 7.99 (dd, J=8.2, 0.5 Hz, 1H), 7.72 (dd, J=9.4, 1.8 Hz, 1H), 7.57 (dt, J=8.2, 0.7 Hz, 1H), 7.47 (td, J=7.9, 1.1 Hz, 1H), 7.31 (td, J=7.9, 1.1 Hz, 1H).

ASAP Mass Spectrometry: theoretical value 354.48, observed value 355.23.

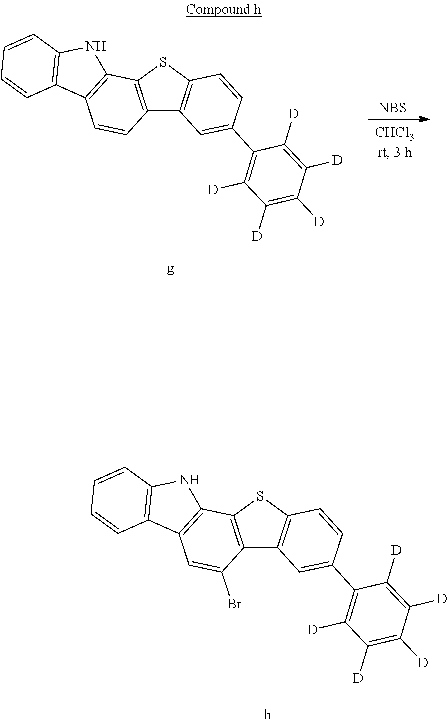 [SMILES: [2H]c1c([2H])c([2H])c(-c2ccc3sc4c(ccc5c6ccccc6[nH]c54)c3c2)c([2H])c1[2H] ; "NBS" ; "CHCl3rt, 3 h" ; [2H]c1c([2H])c([2H])c(-c2ccc3sc4c5[nH]c6ccccc6c5cc(Br)c4c3c2)c([2H])c1[2H]]

Under a nitrogen atmosphere, chloroform (100 mL) was added to compound g (2.00 g, 5.64 mmol), cooled down to 0° C., then N-bromosuccinimide (1.00 g, 5.64 mmol) was added thereto and reacted at room temperature for 3 hours. The resultant reaction solution was extracted and purified by silica gel column chromatography to give compound h (1.06 g, 2.45 mmol, yield 43.4%).

ASAP Mass Spectrometry: theoretical value 432.03, observed value 432.33.

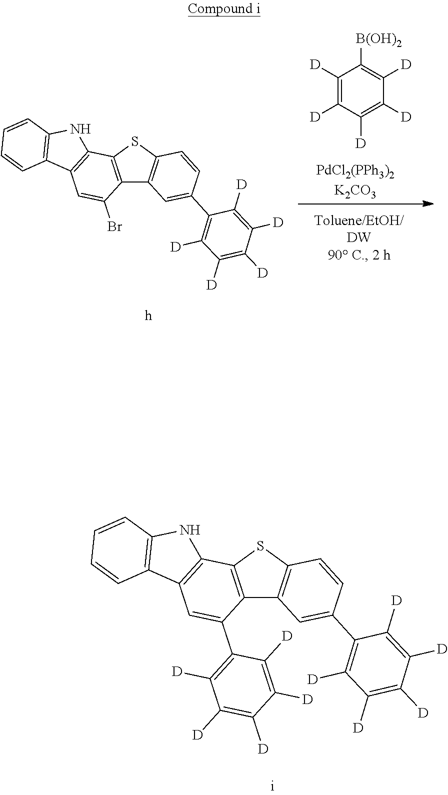 [SMILES: [2H]c1c([2H])c([2H])c(-c2ccc3sc4c5[nH]c6ccccc6c5cc(Br)c4c3c2)c([2H])c1[2H] ; [2H]c1c([2H])c([2H])c(B(O)O)c([2H])c1[2H] ; "Toluene/EtOH/DW90° C., 2 h" ; [2H]c1c([2H])c([2H])c(-c2ccc3sc4c5[nH]c6ccccc6c5cc(-c5c([2H])c([2H])c([2H])c([2H])c5[2H])c4c3c2)c([2H])c1[2H] ; "PdCl2(PPh3)2K2CO3"]

Under a nitrogen atmosphere, a mixed solvent of deaerated toluene (24 mL)/ethanol (16 mL)/water (8 mL) was added to a mixture of compound h (1.00 g, 2.31 mmol), phenyl-d5-boronic acid (0.438 g, 3.44 mmol), dichlorobis(triphenylphosphine) palladium (0.081 g, 0.12 mmol) and potassium carbonate (0.800 g, 5.79 mmol), and reacted at 90° C. for 2 hours. After the reaction, the reaction solution was cooled down to room temperature and extracted. The resultant reaction mixture was purified by silica gel column chromatography to give compound i (0,960 g, 2.20 mmol, yield 95.2%).

ASAP Mass Spectrometry: theoretical value 435.61, observed value 436.45.

¹H NMR (400 MHz, CDCl₃) δ 8.33 (s, 1H), 8.11 (d, J=8.0, 1H), 8.00 (s, 1H), 7.95 (d, J=8.5, 1H), 7.68-7.38 (m, 5H),

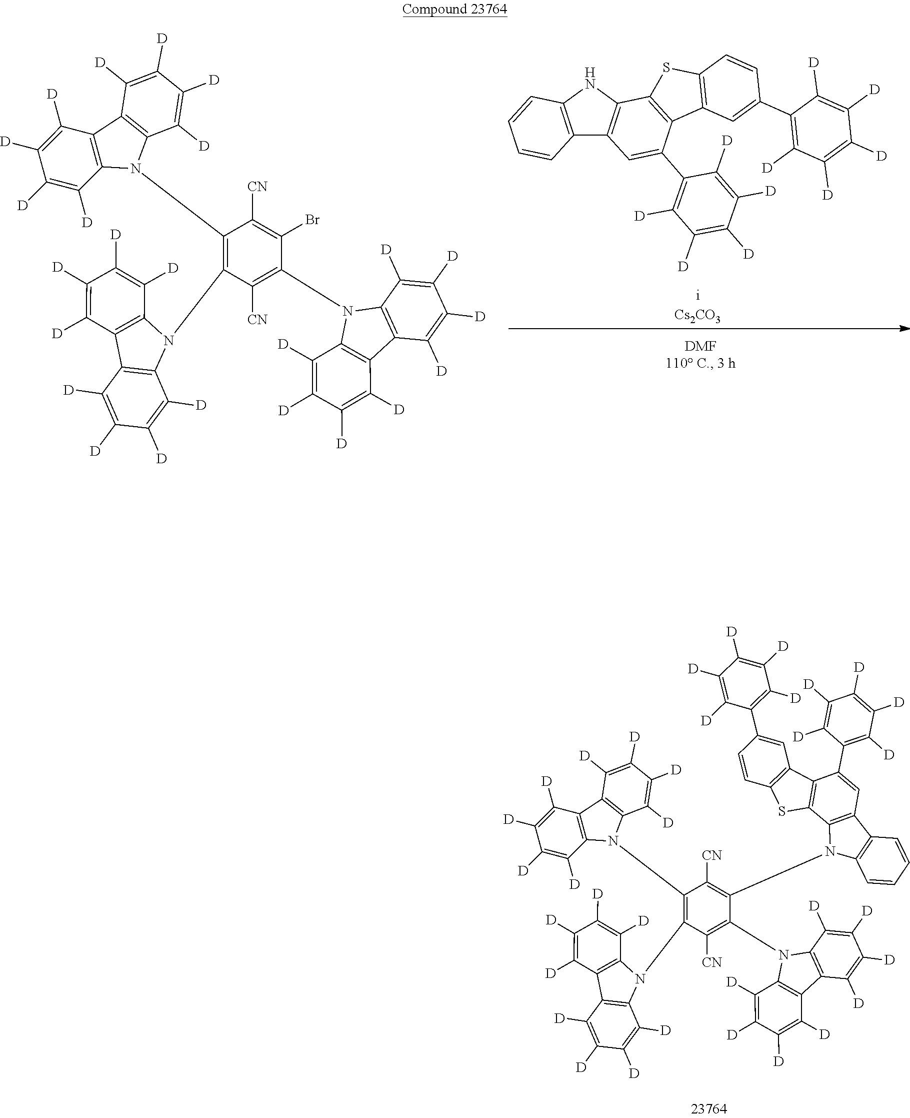

Under a nitrogen atmosphere, dimethylformamide (100 mL) was added to a mixture of compound i (0.506 g, 1.16 mmol), 2-bromo-3,5,6-tris(9H-carbazol-9-yl-d8) terephthalonitrile (0.650 g, 0.89 mmol) and cesium carbonate (0.35 g, 1.07 mmol), and stirred at 110° C. for 3 hours. After the reaction, the reaction solution was cooled down to room temperature, and water was added thereto to stop the reaction. The precipitated solid was recovered by filtration, and washed with methanol. The resultant solid was purified by silica gel column chromatography to give Compound 23764 (0.600 g, 0.555 mmol, yield 62.0%).

¹H NMR (400 MHz, CDCl₃) δ 8.06 (dd, J=8.24, 0.7 Hz, 1H), 7.77 (dd, J=8.24, 1.8 Hz, 1H), 7.71 (s, 1H), 7.39-7.22 (m, 2H), 7.10-6.93 (m, 3H).

ASAP Mass Spectrometry: theoretical value 1081.46, observed value 1082.13.

### (Synthesis Example 4) Synthesis of Compound 23782

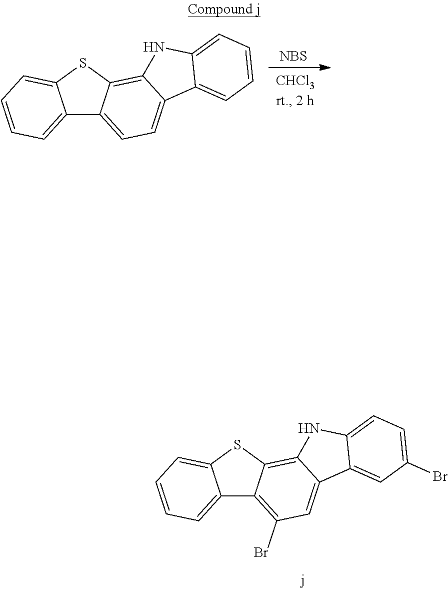

Under a nitrogen atmosphere, 12H-[1]benzothieno[2,3-a]carbazole (5.17 g, 18.9 mmol), N-bromosuccinimide (6.73 g, 37.8 mmol) and chloroform (200 mL) were put into a 300-mL three-neck flask, and stirred at room temperature for 2 hours. After stirred, water was added to the reaction solution for filtration and extraction. The extracted reaction mixture was purified by silica gel column chromatography to give compound j (5.20 g, 12.1 mmol, yield 64%).

¹H NMR (400 MHz, CDCl₃) δ 9.35-9.32 (m, 1H), 8.31-8.25 (m, 2H), 8.20 (d, J=1.8 Hz, 1H), 7.95-7.93 (m, 1H), 7.59-7.52 (m, 3H), 7.41 (d, J=8.6 Hz, 1H)

ASAP Mass Spectrometry: theoretical value 428.88, observed value 430.99.

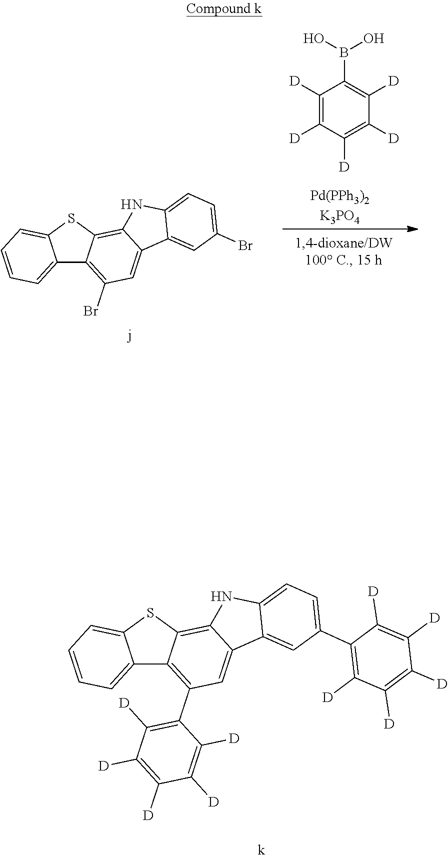 [SMILES: [2H]c1c([2H])c([2H])c(B(O)O)c([2H])c1[2H] ; Brc1ccc2[nH]c3c(cc(Br)c4c5ccccc5sc34)c2c1 ; "1,4-dioxane/DW100° C., 15 h" ; [2H]c1c([2H])c([2H])c(-c2ccc3[nH]c4c(cc(-c5c([2H])c([2H])c([2H])c([2H])c5[2H])c5c6ccccc6sc45)c3c2)c([2H])c1[2H] ; "Pd(PPh3)2K3PO4"]

Under a nitrogen atmosphere, compound j (5.15 g, 11.9 mmol), phenyl-d5-boronic acid (3.64 g, 28.7 mmol), tetrakis(triphenylphosphine) palladium (0) (1.04 g, 0.896 mmol), tripotassium phosphate (7.61 g, 35.8 mmol) and a mixed solvent of 1,4-dioxane (120 mL)/water (40 mL) were put into a 300-mL three-neck flask, and stirred at 100° C. for 15 hours. After stirred, the reaction solution was cooled down to room temperature, and water was added thereto for filtration and extraction. The resultant reaction mixture was purified by silica gel column chromatography to give compound k (2.32 g, 5.33 mmol, yield 45%).

¹H NMR (400 MHz, CDCl₃) δ 8.35 (s, 1H), 8.31 (d, J=1.6 Hz, 1H), 8.00 (d, J=0.7 Hz, 1H), 7.92-7.90 (m, 1H), 7.73 (dd, J=8.4, 1.7 Hz, 1H), 7.63 (dd, J=8.5, 0.7 Hz, 1H), 7.38-7.34 (m, 1H), 7.21-7.18 (m, 1H), 7.15-7.10 (m, 1H)

ASAP Mass Spectrometry: theoretical value 435.19, observed value 436.37.

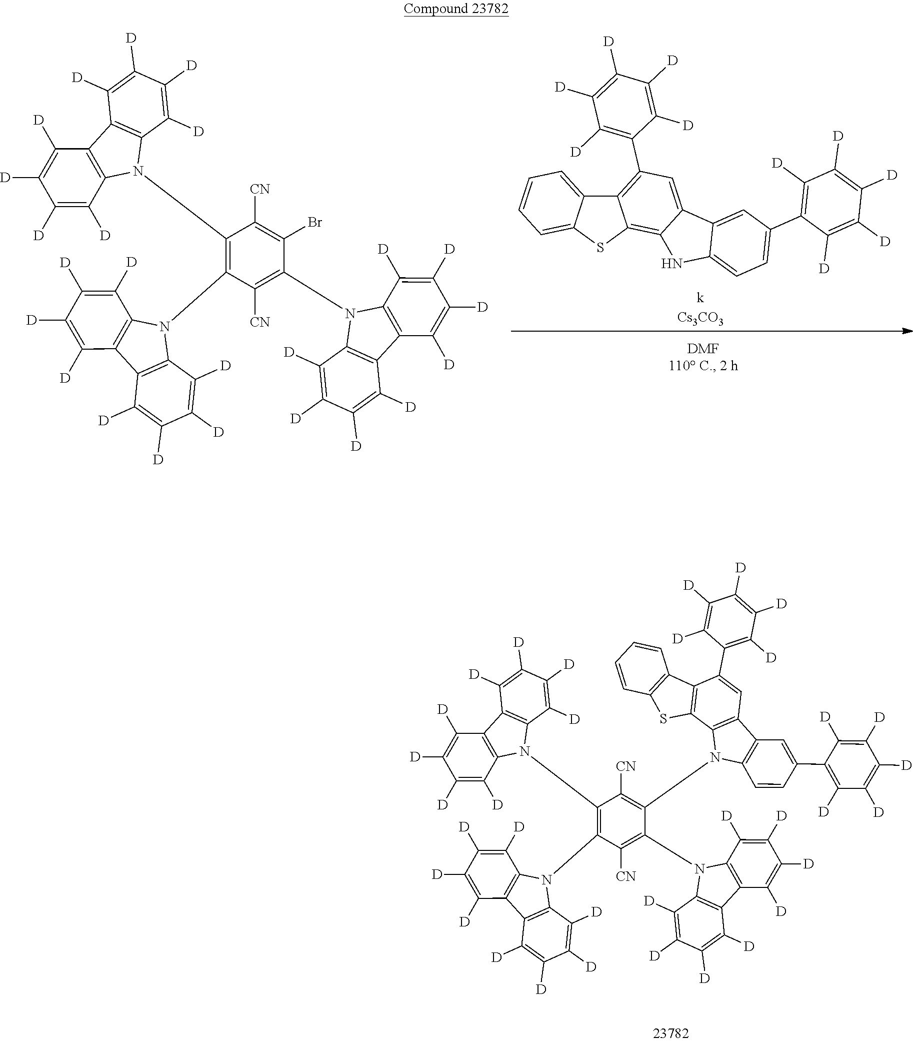

Under a nitrogen atmosphere, 2-bromo-3,5,6-tris(9H-carbazol-9-yl-d8) terephthalonitrile (0.650 g, 0.894 mmol), compound k (0.584 g, 1.34 mmol), cesium carbonate (0.408 g, 1.25 mmol) and dimethylformamide (20 mL) were put into a 100-mL three-neck flask, then stirred at 110° C. for 2 hours, and thereafter cooled down to room temperature. Water was added to the reaction solution, the precipitated solid was recovered by filtration, and washed with methanol. The resultant solid was purified by silica gel column chromatography to give Compound 23782 (0.50 g, 0.46 mmol, yield 52%).

¹H NMR (400 MHz, CDCl₃) δ 8.01 (d, J=7.8 Hz, 1H), 7.84 (d, J=1.5 Hz, 1H), 7.68 (s, 1H), 7.50-7.46 (m, 1H), 7.22-7.13 (m, 3H), 7.05 (d, J=8.5 Hz, 1H)

ASAP Mass Spectrometry: theoretical value 1080.53, observed value 1082.08.

### (Comparative Synthesis Example 1) Synthesis of Comparative Compound C1

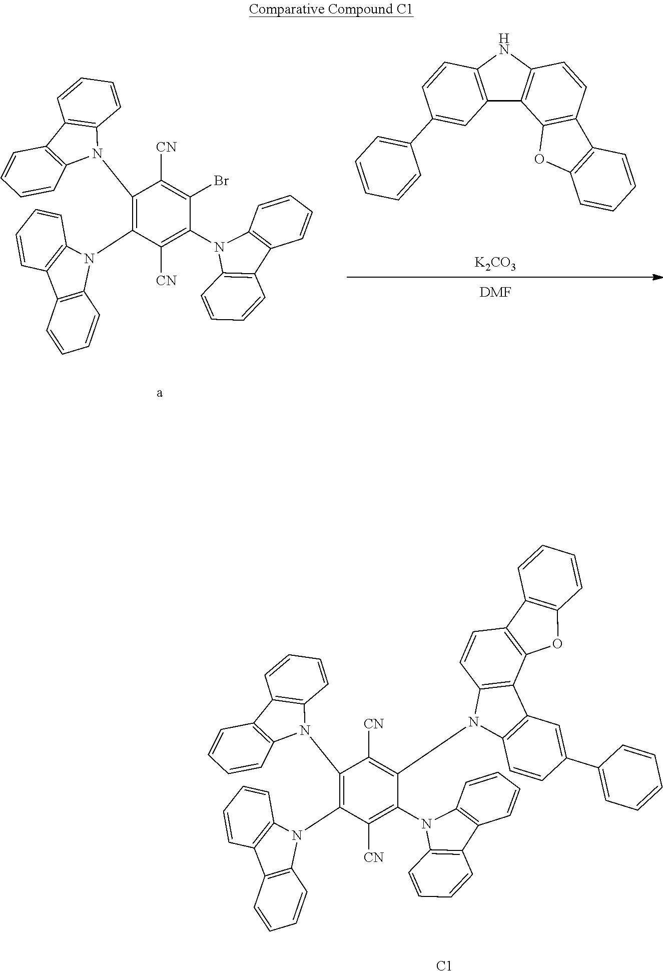

Under a nitrogen stream, an N,N-dimethylformamide solution (20 mL) of Compound a (0.6 g, 0.9 mmol), 2-phenyl-5H-benzofuro[3,2-c]carbazole (0.4 g, 1.1 mmol) and potassium carbonate (0.2 g, 1.3 mmol) was stirred under heat at 120° C. for 7 hours. Thereafter, this was restored to room temperature, then water and methanol were added thereto to precipitate a solid, and the solid was recovered by filtration. The recovered reaction mixture was purified by silica gel column chromatography (toluene/hexane=5/1) and reprecipitation (toluene/methanol) to give Compound C1 (0.6 g, 0.6 mmol, yield 75%).

¹H NMR (400 MHz, CDCl₃) δ 8.38 (s, 1H), 7.97 (d, J=8.0 Hz 1H), 7.82-7.67 (m, 10H), 7.53-7.38 (m, 10H), 7.34-7.28 (m, 4H), 7.24-7.13 (m, 10H), 7.07-7.05 (m, 2H).

ASAP Mass Spectrometry: theoretical value 954.31, observed value 955.68.

### (Example 1) Preparation and Evaluation of Thin Film

Compound 1 and mCBP were vapor-deposited from different vapor deposition sources on a quartz substrate by a vacuum deposition method under conditions of a vacuum degree of less than 1×10⁻³ Pa to form a doped thin film having a content of Compound 1 of 20% by weight and a thickness of 100 nm.

A doped thin film was formed in the same manner, using Comparative Compound C1 in place of Compound 1.

The maximum emission wavelength (λmax) was measured when the formed doped thin films were irradiated with excitation light of 300 nm.

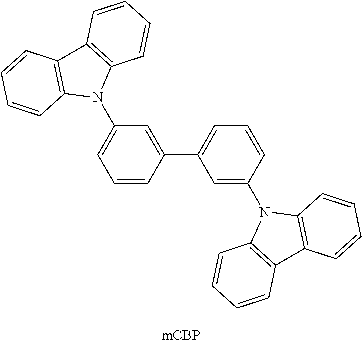 [SMILES: c1cc(-c2cccc(-n3c4ccccc4c4ccccc43)c2)cc(-n2c3ccccc3c3ccccc32)c1]

### (Example 2) Production and Evaluation of Organic Electroluminescent Device Using Compound 1

On a glass substrate on which an anode made of indium-tin oxide (ITO) having a film thickness of 50 nm was formed, each thin film was laminated by a vacuum deposition method at a vacuum degree of 5.0×10⁻⁵ Pa. First, HAT-CN was formed to a thickness of 10 nm on the ITO, NPD was formed to a thickness of 35 nm on the HAT-CN, and further PTCz was formed to a thickness of 10 nm on the NPD. Next, H1, Compound 1 and a light emitting material EM1 were co-deposited from different vapor deposition sources in an amount of 69.5% by weight, 30.0% by weight and 0.5% by weight, respectively, thereby forming a light emitting layer having a thickness of 40 nm. Next, after ET1 was formed to a thickness of 10 nm, Liq and SF3-TRZ were co-deposited from different vapor deposition sources to form a layer with a thickness of 20 nm. The contents of Liq and SF3-TRZ in this layer were 30% by mass and 70% by mass, respectively. Furthermore, Liq was formed to a thickness of 2 nm, and aluminum (Al) was vapor-deposited to a thickness of 100 nm to form a cathode, thereby obtaining an organic electroluminescent device.

An organic electroluminescent device was produced in the same manner using Comparative Compound C1 instead of Compound 1.

Of each organic electroluminescent device, the external quantum efficiency (EQE) at 6.3 mA, and the time taken until the emission intensity at 12.6 mA/cm² reached 95% at the start of the test (LT95) were measured, and the results are shown in Table 5. LT95 is expressed as a relative value when the device using Comparative Compound C1 is defined as 1.

TABLE 5

| | Compound 1 | Comparative Compound C1 |
|---|---|---|
| λmax (nm) | 587.7 | 587.4 |
| EQE (%) | 8.9 | 8.2 |
| LT95 | 1.5 | 1 |

### (Example 3) Preparation and Evaluation of Thin Film

A doped thin film was formed in the same manner as in Example 1, using Compound 23722, 23764 or 23782 in place of Compound 1. The maximum emission wavelength (λmax) was measured when the formed doped thin films were irradiated with excitation light of 300 nm, and the results are shown in Table 6.

### (Example 4) Production and Evaluation of Organic Electroluminescent Devices Using Compound 23722, 23764 or 23782

An organic electroluminescent device was formed in the same manner as in Example 2, using Compound 23722, 23764 or 23782 in place of Compound 1.

The external quantum efficiency (EQE) of each organic electroluminescent device at 6.3 mA was measured, and the results are shown in Table 6.

TABLE 6

| | Compound 23722 | Compound 23764 | Compound 23782 |
|---|---|---|---|
| λmax (nm) | 567.8 | 577.7 | 582.2 |
| EQE(%) | 13.1 | 12.0 | 11.9 |

From the results measured in Examples 1 to 4, it was confirmed that the use of the compound represented by the general formula (1) can improve the light emission efficiency of the devices, can prolong the device lifetime and can improve the durability. Also, it was found that the use of the compound in which the hydrogen atom of the aryl group is substituted with a deuterium atom and the hydrogen atom of the non-ring-fused carbazol-9-yl group is substituted with a deuterium atom can further improve the light emission efficiency of the devices.

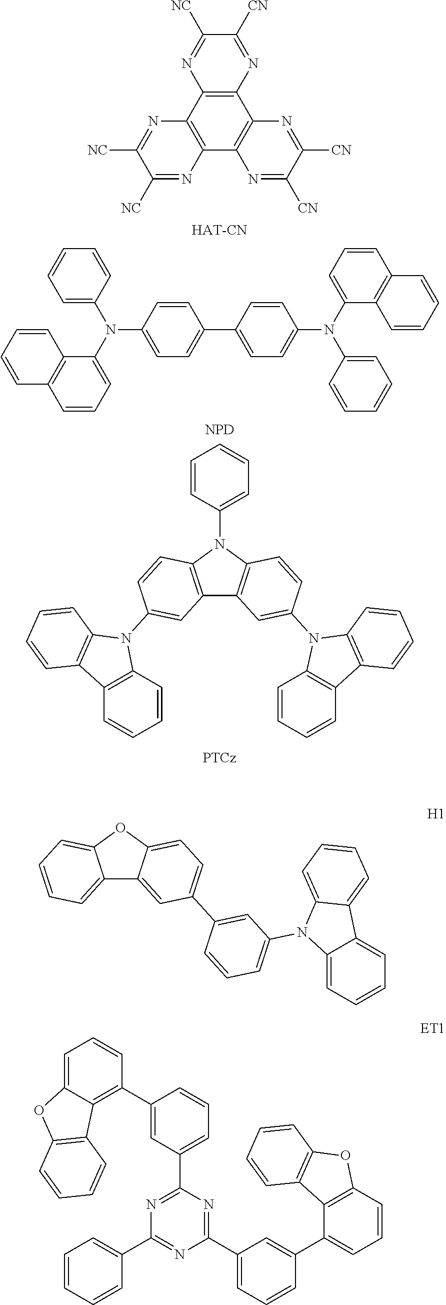

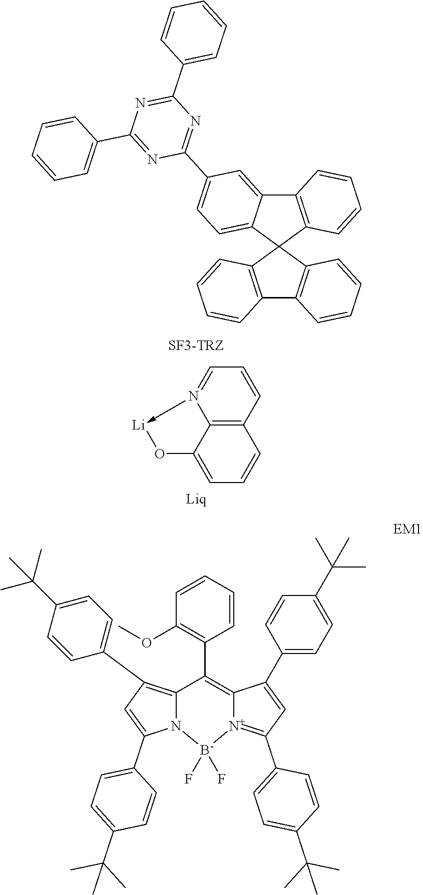

### INDUSTRIAL APPLICABILITY

By using a compound represented by the general formula (1), there can be provided an organic light emitting device having good light emission characteristics. Accordingly, the industrial applicability of the present invention is great.

1. A compound represented by the following general formula (1): 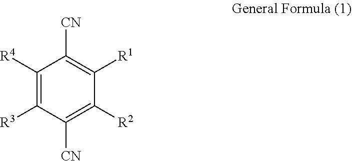
wherein R¹ and 0 or 1 of R² to R⁴ each independently represent a substituted or unsubstituted ring-fused carbazol-9-yl group;
at least R¹ among them is a ring-fused carbazol-9-yl group in which the ring skeleton-constituting carbon atoms are substituted with at least two substituted or unsubstituted aryl groups; and
the remaining R² to R⁴ each independently represent a substituted or unsubstituted non-ring-fused carbazol-9-yl group.

2. The compound according to claim 1, wherein, when one of R² to R⁴ is a ring-fused carbazol-9-yl group, the ring skeleton-constituting carbon atoms of the ring-fused carbazol-9-yl group are substituted with at least two substituted or unsubstituted aryl groups.

3. The compound according to claim 1, wherein R¹ represents a ring-fused carbazol-9-yl group in which the ring skeleton-constituting carbon atoms are substituted with at least two substituted or unsubstituted aryl groups, and R² to R⁴ each independently represent a substituted or unsubstituted non-ring-fused carbazol-9-yl group.

4. The compound according to claim 4, wherein the ring-fused carbazol-9-yl group in which the ring skeleton-constituting carbon atoms are substituted with at least two substituted or unsubstituted aryl groups has a 5- to 7-membered ring-fused structure including carbazole.

5. The compound according to claim 1, wherein the ring-fused carbazol-9-yl group in which the ring skeleton-constituting carbon atoms are substituted with at least two substituted or unsubstituted aryl groups has a structure represented by the following general formula (2): 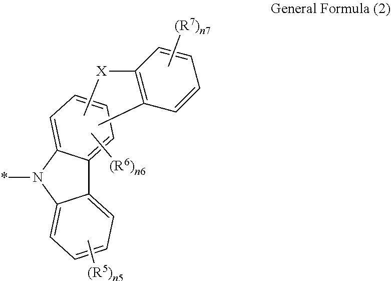
wherein X represents O S, N(R⁸) or C(R⁹)(R¹⁰);
R⁵ to R⁷ each independently represent a substituent, in which the substituent includes a deuterium atom;
n5 and n7 each independently represent an integer of 0 to 4, and n6 represents an integer of 0 to 2;
provided that n5+n6+n7 is an integer of 2 to 10, and at least two of R⁵ to R⁷ existing in the molecule are substituted or unsubstituted aryl groups;
R⁵'s bonding to the neighboring ring skeleton-constituting carbon atoms, R⁶'s bonding to the neighboring ring skeleton-constituting carbon atoms, and R⁷'s bonding to the neighboring ring skeleton-constituting carbon atoms each can bond to each other to form a cyclic structure; and
* indicates the bonding site to the benzene ring in the general formula (1).

6. The compound according to claim 5, wherein n5+n6+n7 is 2.

7. The compound according to claim 5, wherein:
only one of R⁵ and one of R⁷ each are independently a substituted or unsubstituted aryl group, or
only one of R⁵ and one of R⁶ each are independently a substituted or unsubstituted aryl group, or
only one of R⁶ and one of R⁷ each are independently a substituted or unsubstituted aryl group.

8. The compound according to claim 1, wherein the substituted or unsubstituted non-ring-fused carbazol-9-yl group has a structure represented by the following general formula (3): 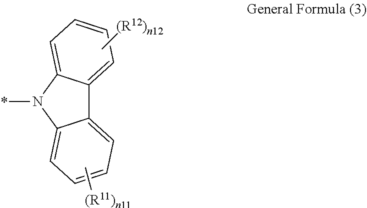
wherein R¹¹ and R¹² each independently represent a substituent, in which the substituent includes a deuterium atom;
n11 and n12 each independently represent an integer of 0 to 4;
one of the 1- to 4-positioned ring skeleton-constituting carbon atoms of the carbazole can be substituted with a nitrogen atom, and one of the 5- to 8-positioned ring skeleton-constituting carbon atoms of the carbazole can be substituted with a nitrogen atom; and
* indicates the bonding site to the benzene ring in the general formula (1).

9. A composition comprising the compound according to claim 1, and a pyrromethene-boron complex compound.

10. A light emitting material comprising the compound according to claim 1.

11. (canceled)

12. A film comprising the compound according to claim 1.

13. An organic semiconductor device comprising the compound according to claim 1.

14. An organic light emitting device comprising the compound according to claim 1.

15. The organic light emitting device according to claim 14, wherein the device has a layer containing the compound, and the layer also contains a host material.

16. The organic light emitting device according to claim 15, wherein the layer containing the compound further contains a delayed fluorescent material in addition to the compound and the host material, and the delayed fluorescent material has a lowest excited singlet energy lower than that of the host material and higher than that of the compound.

17. The organic light emitting device according to claim 15, wherein the device has a layer containing the compound, and the layer also contains a light emitting material having a structure different from that of the compound.

18. The organic light emitting device according to claim 15, wherein the amount of light emitted from the compound is the largest among the materials contained in the device.

19. The organic light emitting device according to claim 17, wherein the amount of light emitted from the light emitting material is larger than the amount of light emitted from the compound.

20. The organic light emitting device according to claim 14, which is an organic electroluminescent device.

21. The organic light emitting device according to claim 14, which emits delayed fluorescence.
